(19) United States
(12) Patent Application Publication
(10) Pub. No.: US 2024/0375106 A1
(43) Pub. Date: Nov. 14, 2024

(54) BLOOD SAMPLE COLLECTION TUBE AND USES THEREOF

(71) Applicant: SiO2 Medical Products, Inc., Aubum, AL (US)

(72) Inventors: Robert S. Abrams, Auburn, AL (US); Zoe Silver, Auburn, AL (US); Marc Tandourjian, Auburn, AL (US); Christopher Weikart, Auburn, AL (US); Tia Hexom, Auburn, AL (US)

(21) Appl. No.: 17/768,035

(22) PCT Filed: Oct. 13, 2020
(86) PCT No.: PCT/US2020/055440

§ 371 Date: Apr. 11, 2022

Related U.S. Application Data
(60) Provisional application No. 62/929,668, filed on Nov. 1, 2019.
(60) Provisional application No. 62/914,142, filed on Oct. 11, 2019.

(51) Int. Cl. B01L 3/00

(52) U.S. Cl. CPC B01L 3/5082; B01L 2300/0832; B01L 2300/16

(57) ABSTRACT

Blood sample collection tubes are described including a tube surface and a coating set comprising a tie coating or layer N of SiOₓC_{y} or SiN_{y}C_{y} applied to the tube surface, a barrier coating or layer of SiOₓ, and a pH protective layer of SiOₓC_{y} or SiN_{y}C_{y}. The C tubes optionally contain a fluid with a pH of 4 to 8, alternatively 5 to 9. The barrier coating or layer prevents oxygen from penetrating into the thermoplastic tube, and the tie coating or layer and pH protective coating or layer together protect the barrier layer from the contents of the tube. Processes of collecting high volumes of blood into the tube, and methods of isolating the various types of nucleic N acids from the collected blood sample are described.

### CROSS-REFERENCE TO RELATED APPLICATION

This application claims the benefit of, priority to, and incorporates by reference in its entirety U.S. Provisional Application No. 62/914,142, filed Oct. 11, 2019, and U.S. Provisional Application No. 62/929,668, filed Nov. 1, 2019.

### FIELD

The present disclosure relates to the technical field of thermoplastic blood collection tubes, for example, such tubes in which blood samples are collected and transported to a medical laboratory for testing. The present disclosure also relates to processes for collecting blood in the thermoplastic blood collection tubes of the disclosure, and methods of isolating nucleic acids from blood collected in the blood collection tubes of the disclosure.

### BACKGROUND OF THE DISCLOSURE

Blood sample collection tubes, are used for drawing blood from a patient for medical analysis. The tubes are commonly sold stoppered and evacuated. The patient's blood is communicated to the interior of a tube by inserting one end of a double-ended hypodermic needle into the patient's blood vessel and impaling the closure of the evacuated blood collection tube on the other end of the double-ended needle. The vacuum in the evacuated blood collection tube draws the blood (or more precisely, the blood pressure of the patient pushes the blood) through the needle into the evacuated blood collection tube, increasing the pressure within the tube and thus decreasing the pressure difference causing the blood to flow. The blood flow typically continues until the tube is removed from the needle or the pressure difference is too small to support flow.

Evacuated blood collection tubes should have a substantial shelf life to facilitate efficient and convenient distribution and storage of the tubes prior to use. For example, a one-year shelf life is desirable, and progressively longer shelf lives, such as 18 months, 24 months, or 36 months, are also desired in some instances. The tube desirably remains essentially fully evacuated, at least to the degree necessary to draw enough blood for analysis (a common standard is that the tube retains at least 90% of the original draw volume), for the full shelf life, with very few (optimally no) defective tubes being provided.

A tube having a lower-than-standard vacuum level at the time of use is likely to cause the phlebotomist using the tube to fail to draw sufficient blood. The phlebotomist might then need to obtain and use one or more additional tubes to obtain a blood sample sufficient for the desired analysis.

To meet this shelf-life requirement, evacuated blood collection tubes have typically been made of glass. Glass vessels have been favored because glass is more gas-tight and inert to prefilled contents than untreated plastics. Also, due to its traditional use, glass is well accepted, as it is known to be relatively innocuous when contacted with blood or other medical samples.

Glass vessels, however, have several serious disadvantages when used as blood tubes. They can break, and if broken, form sharp shards from remnants of the vessel that can injure workers or patients, both due to the direct effects of laceration and by transmitting infections via lacerated skin. Breakage after a sample is collected can also cause the sample to be compromised or lost. Glass vessels also are expensive to manufacture, as glass cannot be injection molded.

Thermoplastic blood collection tubes have been developed to overcome the disadvantages of glass vessels. Plastic vessels are rarely broken in normal use, and if broken, do not form sharp shards from remnants of the vessel, like a glass tube would. As-molded thermoplastic blood collection tubes previously have not had gas barrier properties adequate to provide a commercially desirable shelf life when used as evacuated blood collection tubes. Plastic has allowed small molecule gases to permeate into (or out of) the tube. The permeability of plastics to gases has been significantly greater than that of glass. Many plastics have allowed water vapor to pass through the tubes to a greater degree than glass. Some plastic tubes contain organic or inorganic metal compounds in the plastic that can leach out or be extracted into the sample tube. As a result, plastic vessels have a shortened shelf-life and are limited by additive stability and partial vacuum loss or draw volume consistency. The typical shelf-life of plastic vessels is no more than 18 months, depending on the manufacturer and construction materials.

U.S. Pat. No. 7,985,188 discloses barrier-coated thermoplastic blood collection tubes including a barrier coating or layer applied by plasma-enhanced chemical vapor deposition (PECVD), to improve their gas barrier and leaching properties. An example of a suitable barrier coating disclosed by U.S. Pat. No. 7,985,188 is SiOₓ, in which x in this formula is from about 1.5 to about 2.9, as characterized by x-ray photoelectron spectroscopy (XPS).

Some blood collection tubes, as sold, also contain an aqueous reagent, for example, EDTA, heparin or sodium citrate, to preserve the blood between the times of collection and analysis. Some such reagents can attack and dissolve the barrier coating over time, leading to increased permeation of external atmospheric gas, reduction in the vacuum level, and thus a shorter shelf life of the barrier-coated thermoplastic evacuated blood collection tubes.

Since many of these blood collection tubes are inexpensive and used in large quantities, for certain applications, it will be useful to reliably obtain the necessary shelf life without increasing the manufacturing cost to a prohibitive level.

Thus, there is a desire for thermoplastic blood collection tubes, with gas and solute barrier properties, which approach the properties of glass without unduly increasing the manufacturing cost.

Currently, available blood collection tubes (BCT) are limited for use in collecting blood for nucleic acid analysis. Some tubes and their nucleic acid preservatives are specific for DNA or RNA (intracellular or cell-free). This requires multiple tubes for blood collection and nucleic acid analysis. Others are useful to collect and stabilize both DNA or RNA but require separate purification processes to isolate and purify the DNA or RNA. Others are not useful for high throughput processes.

Thus, there is a need for a universal blood collection tube that facilitates the preservation of the collection of a high volume of a blood sample and the preservation of its nucleic acids. There is also a need for a blood collection tube that can be employed with any downstream chemistry for isolation and purification of such genetic material (intracellular and/or cell-free DNA and/or RNA) and is compatible for use with high-throughput sequencing technology and other large-scale genetic-based testing applications. Such a universal tube and downstream purification process would also enable intrasample comparisons of different types of nucleic acids present within a single sample.

### SUMMARY OF THE DISCLOSURE OF THE APPLICATION

The present application provides novel blood sample collection tubes and uses thereof. In specific embodiments, the application provides blood sample collection tubes for the collection of a high volume of a blood sample containing different types of nucleic acids and the preservation of those nucleic acids, including intracellular DNA and RNA, and cell-free DNA and RNA. The disclosure also provides processes to collect and preserve the blood samples containing DNA and RNA, and methods of isolating the various types of nucleic acids from the collected blood sample for use in high-throughput sequencing and genetic-based testing applications. The present disclosure provides a blood sample collection tube for the collections of blood samples for all 4-types of genetic materials.

The present disclosure provides a sample preparation protocol that enables a single blood sample collection tube to work with any manufacturer's RNA purification kit, for example, Total RNA Purification Kit by Norgen, MasterPure TNA Purification Kit, or GeneJET RNA Purification Kit by Thermo Scientific. The protocol change involves the specific procedures required to prepare the blood sample in the laboratory for sequencing.

In this disclosure, a single blood sample collection tube may be used to collect the blood sample for genetic testing, optionally for all 4-types of genetic tests. This blood sample collection tube contains nucleic acid preservatives. In some embodiments, the blood sample collection tube contains nucleic acid preservatives with a composition of a) at least one volume excluding polymer; (b) at least one osmotic agent; (c) at least one enzyme inhibitor; and (d) optionally, a metabolic inhibitor. In other embodiments the nucleic acid preservative comprises the preservative of paragraph 46, subsections 9-18, 37-46 and 55-60 and paragraphs 92-94. In the case of RNA testing, the collected sample can be purified by any manufacture's RNA purification kit, including those kits that are suitable for high volume sequencing. Such a configuration eliminates the need to use multiple blood collection tubes to collect the needed genetic material, for example, all 4-types of genetic material. The sample preparation process (the purification step) is not limited to one RNA purification kit and any manufacturer's RNA purification kit may be used.

A first embodiment is a blood sample collection tube characterized by a draw volume of about 7.5-9.5 ml comprising a lumen defined at least in part by a wall having an internal and external surface and a coating set on the internal surface. The wall has an interior surface comprising a cyclic olefin polymer (COP) or a cyclic olefin copolymer (COC). The coating set on the surface comprises a tie coating or layer, a barrier coating or layer, and a pH protective coating or layer.

In this first embodiment, the tie coating or layer comprises SiOₓC_{y} or SiNₓC_{y} wherein x is from about 0.5 to about 2.4 and y is from about 0.6 to about 3. The tie coating or layer has an outer surface facing the internal wall surface of the lumen, and the tie coating or layer has an interior surface facing the lumen.

In this first embodiment, the barrier coating or layer comprises SiOx, wherein x is from 1.5 to 2.9, and is from 2 to 1000 nm thick. The barrier coating or layer of SiOx has an outer surface facing the interior surface of the tie coating or layer, and the barrier coating or layer of SiOx has an interior surface facing the lumen. The barrier coating or layer is effective to reduce the ingress of atmospheric gas through the wall compared to an uncoated wall.

In this first embodiment, the pH protective coating or layer comprises SiOₓC_{y} or SiNₓC_{y} wherein x is from about 0.5 to about 2.4 and y is from about 0.6 to about 3. The pH protective coating or layer has an interior surface facing the lumen and an outer surface facing the interior surface of the barrier coating or layer. The pH protective coating or layer is formed by chemical vapor deposition of a precursor selected from an acyclic siloxane, a monocyclic siloxane, a polycyclic siloxane, a polysilsesquioxane, a monocyclic silazane, a polycyclic silazane, a polysilsesquiazane, a silatrane, a silquasilatrane, a silproatrane, an azasilatrane, an azasilquasiatrane, an azasilproatrane, or a combination of any two or more of these precursors.

In this first embodiment, the rate of erosion of the pH protective coating or layer, if directly contacted by a fluid composition having a pH at some point between 5 and 9, is less than the rate of erosion of the barrier coating or layer, if directly contacted by the fluid composition.

In this first embodiment, the blood sample collection tube according to any one of the preceding items, having a shelf life of at least six months, alternatively at least 12 months, alternatively at least 18 months, alternatively 24 months, measured at a temperature of 5 degrees Celsius.

In this first embodiment, the blood sample collection tube is characterized by a water vapor transmission rate (WVTR) in units of mg of moisture per day (mg/day) less than 0.01 mg/day, alternatively less than 0.008 mg/day, alternatively less than 0.005 mg/day, or alternatively less than 0.003 mg/day, as measured with water vapor transmission rate (WVTR), using the formula:

$$WVTR = P \frac{( {p}_{2} - {p}_{1} )}{l} {e}^{- {E}_{A} / RT}$$

where P, is the permeability of water vapor, l, is the thickness, p₂, is the partial pressure of water vapor on one side of the film and p₁ is on the other side, E_{A} is the activation energy, R is the universal gas constant and Tis the temperature.

In this first embodiment, the blood sample collection tube is characterized by an oxygen permeation rate of less than 0.00030/day, alternatively less than 0.00020/day, alternatively less than 0.00018/day, or alternatively less than 0.00018/day measured in a units of time⁻¹ of the article, as measured with oxygen transmission rate (OTR) constant (k_{OTR}), using the formula:

$${k}_{OTR} = \frac{RT}{V} {P}_{article}$$

where V is the BCT volume, P_{article} is the oxygen permeation rate constant of the BCT (with units moles time⁻¹ pressure⁻¹), T is the absolute temperature, and R is the Universal gas constant.

In this first embodiment, the blood sample collection tube is characterized by a tensile strength in a range of about 40 to about 80 MPa, alternatively a range of about 50 to about 70 MPa, alternatively a range of about 55 to about 65 MPa, alternatively a range of about 59 to about 63 MPa.

A second embodiment is a blood sample collection tube comprising a vessel having a lumen defined at least in part by a wall. The wall has an interior surface comprising a cyclic olefin polymer (COP) or a cyclic olefin copolymer (COC) facing the lumen, and the wall has an outer surface. A coating set on the interior surface comprises a tie coating or layer, a barrier coating or layer, and a pH protective coating or layer.

In this second embodiment, the tie coating or layer comprises SiOₓC_{y} or SiNₓC_{y} wherein x is from about 0.5 to about 2.4 and y is from about 0.6 to about 3. The tie coating or layer has an interior surface facing the lumen and an outer surface facing the internal wall surface.

In this second embodiment, the barrier coating or layer comprises SiOₓ, wherein x is from 1.5 to 2.9, from 2 to 1000 nm thick. The barrier coating or layer of SiOₓ has an interior surface facing the lumen and an outer surface facing the interior surface of the tie coating or layer. The barrier coating or layer is effective to reduce the ingress of atmospheric gas into the lumen compared to a vessel without a barrier coating or layer.

In this second embodiment, the pH protective coating or layer comprises SiOₓC_{y} or SiNₓC_{y} wherein x is from about 0.5 to about 2.4 and y is from about 0.6 to about 3. The pH protective coating or layer has an interior surface facing the lumen and an outer surface facing the interior surface of the barrier coating or layer.

In this second embodiment, the combination of the tie coating or layer and the pH protective coating or layer is effective to increase the calculated shelf life of the package (total Si/Si dissolution rate).

In this second embodiment, a fluid composition is contained in the lumen and has a pH between 5 and 9.

In this second embodiment, the calculated shelf life of the package is more than six months at a storage temperature of 4° C.

A third embodiment is a blood sample collection tube comprising a thermoplastic wall, a fluid composition, a tie coating or layer, a barrier coating or layer, and a pH protective coating or layer.

In this third embodiment, the thermoplastic wall has an interior surface comprising a cyclic olefin polymer (COP) or a cyclic olefin copolymer (COC) and encloses a lumen.

In this third embodiment, the fluid composition contained in the lumen has a pH greater than 5 and is disposed in the lumen.

In this third embodiment, the tie coating or layer comprises SiOₓC_{y} or SiNₓC_{y} wherein x is from about 0.5 to about 2.4 and y is from about 0.6 to about 3. The tie coating or layer has an outer surface facing the wall surface, and the tie coating or layer has an interior surface.

In this third embodiment, in the barrier coating or layer of SiOₓ, x is between 1.5 and 2.9. The barrier coating or layer is applied by plasma-enhanced chemical vapor deposition (PECVD). The barrier coating or layer is positioned between the interior surface of the tie coating or layer and the fluid composition, and the barrier coating or layer is supported by the thermoplastic wall. The barrier coating or layer has the characteristic of being subject to being measurably diminished in barrier improvement factor in less than six months as a result of attack by the fluid composition.

In this third embodiment, in the pH protective coating or layer of SiOₓC_{y}, x is between 0.5 and 2.4 and y is between 0.6 and 3. The pH protective coating or layer is applied by PECVD, and the pH protective coating or layer is positioned between the barrier coating or layer and the fluid composition and supported by the thermoplastic wall. The pH protective coating or layer and the tie coating or layer together are effective to keep the barrier coating or layer at least substantially undissolved as a result of attack by the fluid composition for a period of at least six months.

In each of these embodiments, the blood collection tubes of this application have one or more preservatives for preserving nucleic acids. The blood sample collected is particularly useful for genetic-based testing of one or more nucleic acids selected from the group consisting of intracellular DNA, intracellular RNA, cell-free DNA, and cell-free RNA that are present in the blood sample.

A fourth embodiment is a method of isolating at least one of intra-cellular DNA, intracellular RNA, cell-free DNA, and cell-free RNA from a blood sample comprising the steps:
(a) collecting the blood sample from a subject in the collection tube of the present disclosure;
(b) mixing the blood sample with the one or more nucleic acid preservatives contained in the tube within about one minute of collection;
(c) centrifuging the sample obtained in step (b);
(d) separating the supernatant obtained in step (c) from the pellet obtained in step (c), the isolated supernatant containing one or more of the cell-free DNAs or cell-free RNAs in the blood sample in a first solution and the isolated pellet containing anucleated and nucleated cells from the blood sample;
(e) resuspending the pellet obtained in step (d), the resuspension containing one or more of the intra-cellular DNAs or intracellular RNAs in the blood sample in a second buffered solution; and
(f) processing the first solution to isolate the one or more of the cell-free DNAs or cell-free RNAs in the blood sample using any method well-known in the art and/or processing the second solution to isolate the one or more of the intra-cellular DNAs or intracellular RNAs in the blood sample using any method well-known in the art. In this fourth embodiment, the first solution and/or the second solution are further treated with commercially available nucleic acid isolation kits.

In this fourth embodiment, the anucleated cells are red blood cells and the nucleated cells are selected from white blood cells, and tumor cells. The one or more of the isolated cell-free DNA, cell-free RNA, intracellular DNA, and intracellular RNA are useful in high-throughput genetic testing assays.

A fifth embodiment is a process for collecting a blood sample from a subject comprising collecting about 8.1 ml of a blood sample from a subject into a blood collection tube of the disclosure.

A fifth embodiment is a method of isolating at least one of intra-cellular DNA, intracellular RNA, cell-free DNA, and cell-free RNA from a blood sample comprising the steps: (a) collecting the blood sample from a subject in the collection tube of the present disclosure; (b) mixing the blood sample with the one or more nucleic acid preservatives contained in the tube within about one minute of collection; (c) lysing the anucleated cells in the blood sample using a first lysis buffer; (d) centrifuging the sample obtained in step (c); (e) isolating the supernatant obtained in step (d), the supernatant containing one or more of the cell-free DNAs or cell-free RNAs in the blood sample in a first solution; (f) lysing the nucleated cells from the blood sample using a second lysis buffer; (g) centrifuging the sample obtained in step; (h) isolating the supernatant obtained in step (g) containing one or more of the intra-cellular DNAs or intra-cellular RNAs in a second solution.

In this sixth embodiment, the anucleated cells are red blood cells and the nucleated cells are selected from white blood cells, and tumor cells. The one or more of the isolated cell-free DNA, cell-free RNA, intracellular DNA, and intracellular RNA are useful in high-throughput genetic testing assays.

A consistent and reliable blood draw volume is maintained over at least 2 years of shelf-life due to improved gas barrier properties compared to ordinary plastic BCTs. Similarly, moisture vapor loss is so low that preservative evaporation over the BCT's shelf-life is negligible. As a result, the risk of preservative gelation and alteration to the blood-to-preservative ratio mix is practically eliminated.

Further, the hybrid BCTs exhibit superior impact resistance to breakage due to its high ductility and impact strength. This is not influenced by defects and flaws, unlike glass. The COP polymer tolerates cold and hot temperature extremes without mechanical fatigue, deformation or breakage unlike other plastics such as PET.

Particular embodiments of the disclosure are set forth in the following numbered paragraphs:
1. A blood sample collection tube characterized by a draw volume of about 7.5-9.5 ml comprising:
   a lumen defined at least in part by a wall having an internal and external surface and a coating set on the internal surface, the coating set comprising a tie coating or layer, a barrier coating or layer, and a pH protective coating or layer;
      the tie coating or layer comprising SiOₓC_{y} or SiNₓC_{y} wherein x is from about 0.5 to about 2.4 and y is from about 0.6 to about 3, the tie coating or layer having an outer surface facing the internal wall surface of the lumen and the tie coating or layer having an interior surface facing the lumen;
      the barrier coating or layer comprising SiOₓ, wherein x is from 1.5 to 2.9, the barrier coating or layer being from 2 to 1000 nm thick, the barrier coating or layer having an outer surface facing the interior surface of the tie coating or layer and the barrier coating or layer having an interior surface facing the lumen; and
      the pH protective coating or layer comprising SiOₓC_{y} or SiNₓC_{y} wherein x is from about 0.5 to about 2.4 and y is from about 0.6 to about 3, the pH protective coating or layer having an interior surface facing the lumen and an outer surface facing the interior surface of the barrier coating or layer; and
   one or more nucleic acid preservatives.
2. The blood sample collection tube of paragraph 1, wherein the pH protective coating or layer is characterized in that the rate of erosion of the pH protective coating or layer, if directly contacted by a fluid composition having a pH between about 5 and about 9, is less than the rate of erosion of the barrier coating or layer, if directly contacted by the fluid composition.
3. The blood sample collection tube of paragraphs 1 or 2, wherein the pH protective coating or layer is formed by a precursor selected from an acyclic siloxane, a monocyclic siloxane, a polycyclic siloxane, a polysilsesquioxane, a monocyclic silazane, a polycyclic silazane, a polysilsesquiazane, a silatrane, a silquasilatrane, a silproatrane, an azasilatrane, an azasilquasiatrane, an azasilproatrane, or a combination of any two or more of these precursors.
4. The blood sample collection tube of any one of paragraphs 1-3, wherein the pH protective coating or layer is formed by chemical vapor deposition.
5. The blood sample collection tube of paragraph 4, wherein the chemical vapor deposition is plasma enhanced chemical vapor deposition (PECVD).
6. The blood sample collection tube of any one of paragraphs 1-5, wherein the blood sample collection tube has a calculated shelf life (total Silicon/Si dissolution rate) of more than six months at a storage temperature of 4° C.
7. The blood sample collection tube of any one of paragraphs 1-6, wherein the barrier coating or layer is substantially undissolved for a period of at least six months at a storage temperature of 4° C.
8. The blood sample collection tube of any one of paragraphs 1-7, wherein the tube contains about 1.4 ml of the one or more nucleic acid preservatives and is evacuated to about 25-26 mm Hg.
9. The blood sample collection tube of any one of paragraphs 1-8, wherein the nucleic acid preservative is selected from one or more of the group consisting of: ethylenediaminetetraacetic acid (EDTA), K₃EDTA, aurintricarboxylic acid, diazolidinyl urea, dimethoylol-5,5-dimethylhydantoin, dimethylol urea, 2-bromo-2-nitropropane-1,3-diol, oxazolidines, sodium hydroxymethyl glycinate, 5-hydroxymethoxymethyl-1-1aza-3,7-dioxabicyclo octane, 5-hydroxymethyl-1-1 aza-3,7dioxabicyclo octane, 5-hydroxypoly methyl-1-1aza-3,7dioxabicyclo octane, quaternary adamantine, tetraacetic acid (EGTA), 1,2-bis(2-aminophenoxy) ethane-N,N,N′,N′-tetraacetic acid (BAPTA), glycine, imidazolidinylurea, glutathione, lithium chloride, guanidine hydrochloride, urea, spermidine, biuret, and the compositions referred to in International Patent Application PCT/US2011/045405, U.S. Patent Application U.S. Pat. No. 9,012,135, and in U.S. Patent Application publication US 2020/0224189 A1.
10. The blood sample collection tube of paragraph 9, wherein the nucleic acid preservative is a composition comprising:
   (a) a reducing agent;
   (b) a first chaotropic substance;
   (c) a second chaotropic substance;
   (d) a third chaotropic substance;
   (e) a first polyamine substance;
   (f) a second polyamine substance; and
   (g) a chelating agent.
11. The blood sample collection tube of paragraph 10, wherein the reducing agent is glutathione, the first chaotropic substance is LiCI, the second chaotropic substance is guanidine hydrochloride, the third chaotropic substance is urea, the first polyamine substance is spermidine; the second polyamine substance is biuret, and the chelating agent is EDTA.
12. The blood sample collection tube of paragraph 11, wherein the nucleic acid preservative is a composition comprising:
   (a) glutathione in an amount from about 10 mM to about 200 mM;
   (b) LiCI in an amount of from about 1 M to about 4 M;
   (c) guanidine hydrochloride in an amount from about 0.1 M to about 0.9 M;
   (d) urea in an amount from about 2 M to about 12 M;
   (e) spermidine in an amount from about 10 μM to about 300 μM;
   (f) biuret in an amount of from about 10 mM to about 100 mM; and
   (g) EDTA in an amount of from about 1 mM to about 200 mM.
13. The blood sample collection tube of paragraph 9, wherein the nucleic acid preservative is a composition comprising:
   (a) at least one volume excluding polymer;
   (b) at least one osmotic agent;
   (c) at least one enzyme inhibitor; and
   (d) optionally, a metabolic inhibitor.
14. The blood sample collection tube of paragraph 13, wherein the at least one volume excluding polymer is present in an amount of about 10 to about 50% by weight of the composition.
15. The blood sample collection tube of paragraph 13, wherein the at least one enzyme inhibitor is present in an amount of about 1 to about 30% by weight of the composition.
16. The blood sample collection tube of paragraph 13, wherein the at least one osmotic agent is present in an amount of about 1 to about 20% by weight of the composition.
17. The blood sample collection tube of paragraph 13, wherein the metabolic inhibitor is present in an amount of about 0.01 to about 10% by weight of the composition.
18. The blood sample collection tube of paragraph 13, wherein the nucleic acid preservative is a composition comprising:
   (a) the at least one volume excluding polymer is polyethylene glycol (PEG);
   (b) the at least one osmotic agent is NaCl,
   (c) the at least one enzyme inhibitor is EDTA or citrate, and
   (d) the metabolic inhibitor, if present, is sodium azide.
19. The blood sample collection tube of any one of paragraphs 1-18, wherein the pH protective coating or layer as applied has a thickness of between 100 and 700 nm.
20. The blood sample collection tube of any one of paragraphs 1-19, wherein the pH protective coating or layer has a thickness of between 50 and 500 nm two years after the tube is assembled.
21. The blood sample collection tube of any one of paragraphs 1-20, wherein the rate of erosion of the pH protective coating or layer, if directly contacted by a fluid composition having a pH of 8, is less than 20% of the rate of erosion of the barrier coating or layer, if directly contacted by the same fluid composition under the same conditions.
22. The blood sample collection tube of any one of paragraphs 1-21, wherein the rate of erosion of the pH protective coating or layer, if directly contacted by a fluid composition having a pH of 8, is from 5% to 20% of the rate of erosion of the barrier coating or layer, if directly contacted by the same fluid composition under the same conditions.
23. The blood sample collection tube of any one of paragraphs 1-22, wherein an FTIR absorbance spectrum of the pH protective coating or layer has a ratio greater than 0.75 between:
   the maximum amplitude of the Si—O—Si symmetrical stretch peak between about 1000 and 1040 cm⁻¹, and
   the maximum amplitude of the Si—O—Si asymmetric stretch peak between about 1060 and about 1100 cm⁻¹.
24. The blood sample collection tube of paragraph 23, wherein the ratio is no greater than 1.7.
25. The blood sample collection tube of any one of paragraphs 1-24, wherein the silicon dissolution rate of the tube is less than 170 ppb/day as measured in a 50 mM potassium phosphate buffer diluted in water, adjusted to pH 8 with concentrated nitric acid, and containing 0.2 wt. % polysorbate-80 surfactant.
26. The blood sample collection tube of paragraphs 25, wherein the silicon dissolution rate of the tube is less than 160 ppb/day, less than 140 ppb/day, less than 120 ppb/day, less than 100 ppb/day, less than 90 ppb/day, or less than 80 ppb/day.
27. The blood sample collection tube of any one of paragraphs 1-26, wherein the calculated shelf life is more than 18 months.
28. The blood sample collection tube of any one of paragraphs 1-27, wherein the calculated shelf life is more than 2 years.
29. The blood sample collection tube of any one of paragraphs 1-28, wherein the pH protective coating or layer is characterized by an O-Parameter of less than 0.4, as measured with attenuated total reflection (ATR), using the formula:

$$O - Parameter = \frac{Intensity at 1253 {cm}^{- 1}}{Maximum intensity in the range 1000 to 1100 {cm}^{- 1} .}$$

30. The blood sample collection tube of paragraph 29, wherein the O-parameter is between 0.15 and 0.37.
31. The blood sample collection tube of any one of paragraphs 1-30, wherein the pH protective coating or layer is characterized by an N-Parameter of less than 0.7, as measured with attenuated total reflection (ATR) using the formula:

$$N - Parameter = \frac{Intensity at 840 {cm}^{- 1}}{Intensity at 799 {cm}^{- 1}} .$$

32. The blood sample collection tube of paragraph 31, wherein the N-parameter is between 0.4 and 0.6.
33. The blood sample collection tube of any one of paragraphs 1-32, wherein x is between 0.5 and 1.5 and y is between 0.9 and 2 for the pH protective coating or layer.
34. The blood sample collection tube of any one of paragraphs 1-33, wherein the tie coating or layer has an average thickness of between 10 and 100 nm.
35. The blood sample collection tube of any one of paragraphs 1-34, wherein the barrier coating or layer has an average thickness of between 10 and 100 nm.
36. The blood sample collection tube of any one of paragraphs 1-35, wherein the pH protective coating or layer has a range of thickness of between 50 and 400 nm.
37. The blood sample collection tube of any one of paragraphs 1-36, wherein the one or more nucleic acid preservatives is contained in a liquid solution.
38. The blood sample collection tube of any one of paragraphs 1-37, wherein the blood collection tube also contains an anticoagulant.
39. The blood sample collection tube of any one of paragraphs 1-38, wherein the blood collection tube does not contain formaldehyde.
40. The blood sample collection tube of any one of paragraphs 1-39, wherein the one or more nucleic acid preservatives is capable of preserving nucleic acid for about 30 days at room temperature.
41. The blood sample collection tube of any one of paragraphs 1-40, wherein the one or more preservatives is capable of preserving circulating tumor cells in a blood sample for about 14 days at room temperature.
42. The blood sample collection tube of any one of paragraphs 1-41, wherein the one or more preservatives is capable of preserving nucleic acid for at least 8 days at 37° C.
43. The blood sample collection tube of any one of paragraphs 1-42, wherein the nucleic acid is an intra-cell nucleic acid, a cell-free nucleic acid, or a combination thereof.
44. The blood sample collection tube of any one of paragraphs 1-43, wherein the nucleic acid is RNA, DNA or combinations thereof.
45. The blood sample collection tube of any one of paragraphs 1-44, wherein the tube is capable of preserving a blood sample collected in the tube in the context of one or more of the following characteristics:
   (a) less than 5% apoptosis of blood cells;
   (b) less than 5% genomic DNA fragmentation;
   (c) less than 5% hemolysis of blood cells; and
   (d) less than 5% plasma loss during shipping of the tube.
46. The blood sample collection tube of any one of paragraphs 1-45, characterized by a shelf life of at least six months, alternatively at least 12 months, alternatively at least 18 months, alternatively 24 months, measured at a temperature of 25 degrees Celsius.
47. The blood sample collection tube of any one of paragraphs 1-46, characterized by a water vapor transmission rate (WVTR) in units of mg of moisture per day (mg/day) less than 0.01 mg/day, alternatively less than 0.008 mg/day, alternatively less than 0.005 mg/day, or alternatively less than 0.003 mg/day, as measured with water vapor transmission rate (WVTR), using the formula:

$$WVTR = P \frac{( {p}_{2} - {p}_{1} )}{l} {e}^{- {E}_{A} / RT}$$

where P, is the permeability of water vapor, l, is the thickness, p₂, is the partial pressure of water vapor on one side of the film and p₁ is on the other side, E_{A} is the activation energy, R is the universal gas constant and Tis the temperature.
48. The blood sample collection tube of any one of paragraphs 1-47, characterized oxygen permeation rate of less than 0.00030/day, alternatively less than 0.00020/day, alternatively less than 0.00018/day, or alternatively less than 0.00018/day measured in a units of time−1 of the article, as measured with oxygen transmission rate (OTR) constant (k_{OTR}), using the formula:

$${k}_{OTR} = \frac{RT}{V} {P}_{article}$$

where V is the BCT volume, P_{article} is the oxygen permeation rate constant of the BCT (with units moles time⁻¹ pressure⁻¹), T is the absolute temperature, and R is the Universal gas constant.
49. The blood sample collection tube of any one of paragraphs 1-48, characterized by a draw volume that retains at least 85% of an original draw volume for a shelf life of at least 24 months.
50. The blood sample collection tube of any one of paragraphs 1-49, characterized by a draw volume that retains at least 90% of an original draw volume for a shelf life of at least 24 months.
51. The blood sample collection tube of any one of paragraphs 1-50, characterized by a draw volume that retains at least 95% of an original draw volume for a shelf life of at least 24 months.
52. A method of isolating at least one of intra-cellular DNA, intracellular RNA, cell-free DNA, and cell-free RNA from a blood sample comprising the steps:
   (a) collecting the blood sample from a subject in the collection tube of any one of paragraphs 1-51;
   (b) mixing the blood sample with the one or more nucleic acid preservatives contained in the tube within about one minute of collection;
   (c) centrifuging the sample obtained in step (b);
   (d) separating the supernatant obtained in step (c) from the pellet obtained in step (c), the isolated supernatant containing one or more of the cell-free DNAs or cell-free RNAs in the blood sample in a first solution and the isolated pellet containing nucleated cells from the blood sample;
   (e) resuspending the pellet obtained in step (d), the resuspension containing one or more of the intra-cellular DNAs or intracellular RNAs in the blood sample in a second solution; and
   (f) processing the first solution to isolate the one or more of the cell-free DNAs or cell-free RNAs in the blood sample using any method well-known in the art and/or processing the second solution to isolate the one or more of the intra-cellular DNAs or intracellular RNAs in the blood sample using any method well-known in the art.
53. A method of isolating at least one of intra-cellular DNA, intracellular RNA, cell-free DNA, and cell-free RNA from a blood sample comprising the steps:
   (a) collecting the blood sample from a subject in the collection tube of any one of paragraphs 1-51;
   (b) mixing the blood sample with the one or more nucleic acid preservatives contained in the tube within about one minute of collection;
   (c) lysing the anucleated cells in the blood sample using a first lysis buffer;
   (d) centrifuging the sample obtained in step (c);
   (e) isolating the supernatant obtained in step (d), the supernatant containing one or more of the cell-free DNAs or cell-free RNAs in the blood sample in a first solution;
   (f) lysing the nucleated cells from the blood sample using a second lysis buffer;
   (g) centrifuging the sample obtained in step (f);
   (h) isolating the supernatant obtained in step (g) containing one or more of the intra-cellular DNAs or intra-cellular RNAs in a second solution.
54. The method of paragraph 52 or 53, wherein the first solution and/or the second solution are treated with commercially available nucleic acid isolation kits.
55. The method of paragraphs 52-54, wherein the solutions of the one or more of the isolated cell-free DNAs, cell-free RNAs, intracellular DNAs, and intracellular RNAs are capable of being used in high-throughput genetic testing assays.
56. The method of any one of paragraphs 52-55, wherein the one or more nucleic acid preservatives is a composition comprising
   (a) at least one volume excluding polymer;
   (b) at least one osmotic agent;
   (c) at least one enzyme inhibitor; and
   (d) optionally, a metabolic inhibitor.
57. The method of paragraph 56, wherein the at least one volume excluding polymer is present in an amount of about 10 to about 50% by weight of the composition.
58. The method of paragraph 56, wherein the at least one enzyme inhibitor is present in an amount of about 1 to about 30% by weight of the composition.
59. The method of paragraph 56, wherein the at least one osmotic agent is present in an amount of about 1 to about 20% by weight of the composition.
60. The method of paragraph 55, wherein the metabolic inhibitor is present in an amount of about 0.01 to about 10% by weight of the composition.
61. The method of paragraph 55, wherein the one or more nucleic acid preservatives is a composition comprising:
   (a) the at least one volume excluding polymer is polyethylene glycol (PEG);
   (b) the at least one osmotic agent is NaCl,
   (c) the at least one enzyme inhibitor is EDTA or citrate, and
   (d) the metabolic inhibitor, if present, is sodium azide.
62. A process for collecting a blood sample from a subject comprising collecting about 8.1 ml of a blood sample from a subject into a blood collection tube of any one of paragraphs 1-51.

### BRIEF DESCRIPTION OF THE DRAWINGS

FIG. 1 shows a plastic vessel of any type provided with a trilayer coating according to any embodiment.

FIG. 2 is a detail view of the plastic vessel of FIG. 1.

FIG. 3 corresponds to FIG. 23 of U.S. Pat. No. 7,985,188 and shows a vessel 268 configured as an evacuated blood collection tube according to any embodiment. In FIG. 3 the closure 270 is an assembly of a stopper and a shield with the vessel 268.

FIG. 4 is a plot of silicon dissolution versus exposure time at pH 6 for a glass container versus a plastic container having an SiOₓ barrier layer coated in the inside wall according to any embodiment.

FIG. 5 is a plot of silicon dissolution versus exposure time at pH 7 for a glass container versus a plastic container having an SiOₓ barrier layer coated in the inside wall according to any embodiment.

FIG. 6 is a plot of silicon dissolution versus exposure time at pH 8 for a glass container versus a plastic container having an SiOₓ barrier layer coated in the inside wall according to any embodiment.

FIG. 7 is a plot of the SiOₓ coating thickness necessary initially to leave a 30 nm residual coating thickness when stored with solutions at different nominal pH values from 3 to 9 according to any embodiment.

FIG. 8 shows the silicon dissolution rates at pH 8 and 40° C. of various PECVD coatings according to any embodiment.

FIG. 9 is a plot of the ratio of Si—O—Si symmetric/asymmetric stretching mode versus energy input per unit mass (W/FM or KJ/kg) of a PECVD coating using as the reactive precursor gases OMCTS and oxygen according to any embodiment.

FIG. 10 is a plot of silicon shelf life (days) versus energy input per unit mass (W/FM or KJ/kg) of a PECVD coating using as the reactive precursor gases OMCTS and oxygen. According to any embodiment

FIG. 11 is a Fourier Transform Infrared Spectrophotometer (FTIR) absorbance spectrum of a PECVD coating according to any embodiment.

FIG. 12 is a Fourier Transform Infrared Spectrophotometer (FTIR) absorbance spectrum of a PECVD coating according to any embodiment.

FIG. 13 is a Fourier Transform Infrared Spectrophotometer (FTIR) absorbance spectrum of a PECVD coating according to any embodiment.

FIG. 14 is a Fourier Transform Infrared Spectrophotometer (FTIR) absorbance spectrum of a PECVD coating according to any embodiment.

FIG. 15 is a Fourier Transform Infrared Spectrophotometer (FTIR) absorbance spectrum of a PECVD coating, originally presented as FIG. 5 of U.S. Pat. No. 8,067,070, annotated to show the calculation of the O-Parameter referred to in that patent, according to any embodiment.

FIG. 16 is a schematic view of a syringe with a trilayer coating according to FIGS. 1, 2, and 3, showing a cylindrical region and specific points where data was taken, according to any embodiment.

FIG. 17 is a Trimetric map of the overall trilayer coating thickness versus position in the cylindrical region of a syringe illustrated by FIGS. 16, 1, and 2, representing a vessel according to any embodiment.

FIG. 18 is a photomicrographic sectional view showing the substrate and coatings of the trilayer coating at position 2 shown in FIG. 16, according to any embodiment.

FIG. 19 is another Trimetric map of the overall trilayer coating thickness versus position in the cylindrical region of a vessel illustrated by FIGS. 16, 1, and 2, according to any embodiment.

FIG. 20 is a plot of coating thickness, representing the same coating as FIG. 19, at Positions 1, 2, 3, and 4 shown in FIG. 16, according to any embodiment.

FIG. 21 is a schematic illustration of a vessel, showing points on its surface where measurements were made in a working example, according to any embodiment.

FIG. 22 is a photograph showing the benefit of the present trilayer coating in preventing pinholes after attack by an alkaline reagent, as discussed in the working examples, according to any embodiment.

FIG. 22A is an enlarged detail view of the indicated portion of FIG. 22, according to any embodiment.

FIG. 23 is a view similar to FIG. 3 showing a vessel 268 configured as an evacuated blood collection tube according to any embodiment containing a fluid 218.

FIG. 24 shows a process for making the blood collection tubes in a view similar to tube in FIG. 3 showing a vessel 268 configured as an evacuated blood collection tube according to any embodiment.

FIG. 25 shows the blood collection tube with the coating composed of three individual layers each with a specific function, according to any embodiment.

FIG. 26 shows a diagram of a comparison of the oxygen transmission rate OTR constant for three blood collection tube (BCTs), specifically, a cyclic olefin polymer (COP) BCT, a barrier coated cyclic olefin polymer (COP) BCT, a polyethylene terephthalate (PET) BCT and glass 9 ml blood collection tube (BCT).

FIG. 27 shows a linear regression analysis of a draw volume over time, as applied to the data for each of the three BCT types of FIG. 26.

FIG. 28 shows a diagram of draw volume for two sizes of BCT over the shelf life, according to any embodiment.

FIG. 29 shows a water vapor transmission rate (WVTR) for the cyclic olefin polymer (COP) and polyethylene terephthalate (PET).

FIGS. 30A-30E are spectrograms showing the relative amounts of cfDNA (signal peaks at ca. 58 sec) and contaminating gDNA (signal peaks between about 90-120 sec) in blood samples, stored for a period of up to 28 days at room temperature. DNA was isolated from blood collected into a blood sample collection tube containing a preservative composition as disclosed herein.

FIG. 31 shows a diagram of calculated and observed draw volume for blood sample collection tubes over 18 months, according to any embodiment.

The following reference characters are used in the drawing figures according to any embodiment:

| | |
|---|---|
| 210 | Pharmaceutical package |
| 212 | Lumen |
| 214 | Wall |
| 218 | Fluid |
| 268 | Vessel |
| 270 | Closure |
| 274 | Lumen |
| 285 | Vessel coating or layer set |
| 285a | Closure coating or layer set (inner surface) |
| 285b | Closure coating or layer set (side surface) |
| 286 | pH protective coating or layer |
| 288 | Barrier layer |
| 289 | Tie coating or layer |

### Definitions

In the context of the present disclosure, the following definitions and abbreviations are used:

The word “comprising” according to any embodiment does not exclude other elements or steps, and the indefinite article “a” or “an” does not exclude a plurality unless indicated otherwise. Whenever a parameter range is indicated, it is intended to disclose the parameter values given as limits of the range and all values of the parameter falling within said range. Reference to “about” a value or parameter herein includes (and describes) embodiments that are directed to that value or parameter per se. For example, description referring to “about X” includes description of “X.” Numeric ranges are inclusive of the numbers defining the range. As used herein, the term “about” permits a variation of ±10% within the range of the significant digit.

Where aspects or embodiments are described in terms of a Markush group or other grouping of alternatives, the present application encompasses not only the entire group listed as a whole, but each member of the group individually and all possible subgroups of the main group, and also the main group absent one or more of the group members. The present application also envisages the explicit exclusion of one or more of any of the group members in the embodiment disclosure.

Exemplary methods and materials are described herein, although methods and materials similar or equivalent to those described herein can also be used in the practice or testing of the various aspects and embodiments. The materials, methods, and examples are illustrative only and not intended to be limiting.

In order that the disclosure may be more readily understood, certain terms are first defined. These definitions should be read in light of the remainder of the disclosure and as understood by a person of ordinary skill in the art. Unless defined otherwise, all technical and scientific terms used herein have the same meaning as commonly understood by a person of ordinary skill in the art. Additional definitions are set forth throughout the detailed description.

“First” and “second” or similar references to, for example, deposits of coatings or layers, processing stations or processing devices according to any embodiment refer to the minimum number of deposits, processing stations or devices that are present, but do not necessarily represent the order or total number of deposits, processing stations and devices or require additional deposits, processing stations and devices beyond the stated number. These terms do not limit the number of processing stations or the particular processing carried out at the respective stations. For example, a “first” deposit in the context of this specification can be either the only deposit or any one of plural deposits, without limitation. In other words, recitation of a “first” deposit allows but does not require an embodiment that also has a second or further deposit.

For purposes of the present disclosure according to any embodiment, an “organosilicon precursor” is a compound having at least one of the linkages:

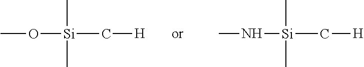

which is a tetravalent silicon atom connected to an oxygen or nitrogen atom and an organic carbon atom (an organic carbon atom being a carbon atom bonded to at least one hydrogen atom). A volatile organosilicon precursor, defined as such a precursor that can be supplied as a vapor in a PECVD apparatus, is an optional organosilicon precursor. Optionally, the organosilicon precursor is a linear siloxane or a monocyclic siloxane, or a combination of any two or more of these precursors.

A “vessel” in the context of the present disclosure can be any type of vessel with at least one opening and a wall defining an inner or interior surface, according to any embodiment. The substrate can be the wall of a vessel having a lumen, according to any embodiment. A preferred “vessel” in the context of the present disclosure is a “blood collection tube”.

The term “at least” in the context of the present disclosure according to any embodiment means “equal or more” than the integer following the term. Thus, a vessel in the context of the present disclosure has one or more openings. One or two openings, like the openings of a sample tube (one opening) or a vessel (two openings) are preferred. A vessel according to the present disclosure can be a sample tube, for example for collecting or storing biological fluids like blood according to any embodiment.

A vessel according to any embodiment can be of any shape, a vessel having a substantially cylindrical wall adjacent to at least one of its open ends being preferred. Generally, the interior wall of the vessel is cylindrically shaped, like, for example, in a sample tube. Sample tubes are contemplated, according to any embodiment.

The values of w, x, y, and z according to any embodiment are applicable to the empirical composition Si_{W}OₓC_{y}H_{z} throughout this specification. The values of w, x, y, and z used throughout this specification should be understood as ratios or an empirical formula (for example, for a coating or layer), rather than as a limit on the number or type of atoms in a molecule. For example, octamethylcyclotetrasiloxane, which has the molecular composition Si₄O₄C₈H₂₄, can be described by the following empirical formula, arrived at by dividing each of w, x, y, and z in the molecular formula by 4, the largest common factor: Si₁O₁C₂H₆. The values of w, x, y, and z are also not limited to integers according to any embodiment. For example, (acyclic) octamethyltrisiloxane, molecular composition Si₃O₂C₈H₂₄, is reducible to Si₁O_{0.67}C_{2.67}H₈. Also, although SiOₓC_{y}H_{z} is described as equivalent to SiOₓC_{y} according to any embodiment, it is not necessary to show the presence of hydrogen in any proportion to show the presence of SiOₓC_{y}.

The atomic ratio according to any embodiment can be determined by XPS. Taking into account the H atoms, which are not measured by XPS, the coating or layer may thus in one aspect have the formula Si_{W}OₓC_{y}H_{z} (or its equivalent SiOₓC_{y}), for example where w is 1, x is from about 0.5 to about 2.4, y is from about 0.6 to about 3, and z is from about 2 to about 9.

As used herein, the term “nucleic acid” in the context of the present disclosure can be, for example, deoxyribonucleic acid (DNA), and/or ribonucleic acid (RNA), and further includes DNA and/or RNA which is linear or branched, single or double-stranded or fragments thereof. The nucleic acids may be contained within a cell (i.e., intracellular), such as a white blood cell or a circulating tumor cell in blood, or they may be present, independent of cells, in biological fluids such as plasma. “Cell-free” nucleic acids are DNA and RNA molecules released by cells into the circulation and transported in the plasma within microvesicles, exosomes, or bound to high-density lipoproteins (HDLs). “Cell-free” nucleic acids include microRNAs.

As used herein, the term “preservative” in the context of the present disclosure can be any type of preservative that maintains the integrity of nucleic acids in the cells and/or nucleic acids in the blood sample. The preservative may stabilize nucleic acids and protect them from degradation, such that the nucleic acids can later be isolated from the blood sample and analyzed using conventional molecular biology techniques. Nucleic acids preserved using the nucleic acid preservative in the blood collection tube of the present disclosure can be isolated from the blood sample following extended periods of storage over a range of temperatures and can be used in diagnostic and sequencing applications.

In some embodiments, the preservative is a nucleic acid preservative. The nucleic acid preservative can preserve one or all intracellular or cell-free DNAs and/or RNAs. In some embodiments, the preservative does not contain formaldehyde. In some embodiments, the preservative may preserve the nucleic acid for about 30 days at room temperature. In other embodiments, the preservative may preserve the circulating tumor cell for about 14 days at room temperature.

In some embodiments, the nucleic acid preservative is selected from the group consisting of one or more of the following components: ethylenediaminetetraacetic acid (EDTA), K₃EDTA, aurintricarboxylic acid, diazolidinyl urea, dimethoylol-5,5-dimethylhydantoin, dimethylol urea, 2-bromo-2-nitropropane-1,3-diol, oxazolidines, sodium hydroxymethyl glycinate, 5-hydroxymethoxymethyl-1-1aza-3,7-dioxabicyclo octane, 5-hydroxymethyl-1-1 aza-3,7dioxabicyclo octane, 5-hydroxypoly methyl-1-1aza-3,7dioxabicyclo octane, quaternary adamantine, tetraacetic acid (EGTA), 1,2-bis(2-aminophenoxy) ethane-N,N,N′,N′-tetraacetic acid (BAPTA), glycine, imidazolidinylurea, and the compositions referred to in International Patent Application PCT/US2011/045405, U.S. Patent Application U.S. Pat. No. 9,012,135 and U.S. Patent Published Application US 2020/0224189 A1. In some embodiments, the preservative is the composition referred to in U.S. Patent Application U.S. Pat. No. 9,012,135. In some embodiments, the preservative is the composition referred to in U.S. Patent Published Application US 2020/0224189 A1. In some embodiments, the nucleic acid preservative prevents genomic DNA release and lysis, stabilizes cellular DNA or RNA, and stabilizes cell-free DNA or RNA. In some embodiments, the nucleic acid preservative is a composition comprising or more of the following components: a reducing agent, a chaotropic substance; a polyamine substance; and a chelating agent. In some embodiments, the nucleic acid preservative is a composition comprising glutathione, LiCI, guanidine hydrochloride, urea, spermidine, biuret, and EDTA. In some embodiments, the blood collection tube of the disclosure is capable of preserving a blood sample collected in the tube in the context of one or more of the following characteristics: (a) less than 5% apoptosis of blood cells; (b) less than 5% genomic DNA fragmentation; (c) less than 5% hemolysis of blood cells; and (d) less than 5% plasma loss during shipping of the tube.

In some embodiments, the preservative is the composition referred to in U.S. Patent Published Application US 2020/0224189 A1. In some embodiments, the nucleic acid preservative prevents genomic DNA release and lysis, stabilizes cellular DNA or RNA, and stabilizes cell-free DNA or RNA. In some embodiments, the nucleic acid preservative is a composition comprising or more of the following components: a volume excluding polymer, an osmotic agent, and an enzyme inhibitor. In some embodiments, the nucleic acid preservative is a composition comprising polyethylene glycol (PEG), NaCl, and EDTA. In some embodiments, the nucleic acid preservative is a composition comprising polyethylene glycol (PEG), NaCl, and a citrate. In some embodiments, the composition further comprises a metabolic inhibitor. In some embodiments, the composition further comprises sodium azide. In some embodiments, the blood sample collection tube of the present disclosure is capable of preserving a blood sample collected in the tube in the context of one or more of the following characteristics: (a) less than 5% apoptosis of blood cells; (b) less than 5% genomic DNA fragmentation; (c) less than 5% hemolysis of blood cells; and (d) less than 5% plasma loss during shipping of the tube.

As used herein, the term “genetic-based testing” in the context of the present disclosure refers to high-throughput automated sequencing and genotyping arrays, next-generation sequencing, multiplexed genotyping, multiplex polymerase chain reaction, non-invasive prenatal testing, and the like. Examples of such techniques are described in Rodriguez-Lee M et al., (2018) Arch Pathol Lab Med 142:199-207; Wong D et al., (2013) Clin Biochem 46(12):1099-1104; Merker J D et al., (2018) J Clin Oncol. 36(16):1631-1641; and Huang L H et al., (2017), PLoS ONE 12(9):e0184692.

As used herein, the term “isolating” refers to the procedure for separating the various types of nucleic acids present in a blood sample collected in a blood collection tube of the present disclosure. The compositions and methods for isolating nucleic acids from a blood sample may include buffers such as lysis buffer, binding buffers, stabilizing buffers, resuspension buffers and the like. The process of isolating may include steps such as centrifugation, extraction, precipitation, dilution, binding, elution, and chromatography.

As used herein, the term “EDTA” in the context of the present disclosure can be EDTA disodium salt, EDTA trisodium salt, EDTA tetrasodium salt, EDTA dipotassium salt, EDTA tripotassium salt, and any combinations thereof.

### DETAILED DESCRIPTION

The present disclosure will now be described more fully, with reference to the accompanying drawings, according to any embodiment. This disclosure can, however, be embodied in many different forms and should not be construed as limited to the embodiments set forth here. Rather, these embodiments are examples, which has the full scope indicated by the language of the claims. Like numbers refer to like or corresponding elements throughout. The following disclosure relates to all embodiments unless specifically limited to a certain embodiment.

### Vessels and Coating Sets

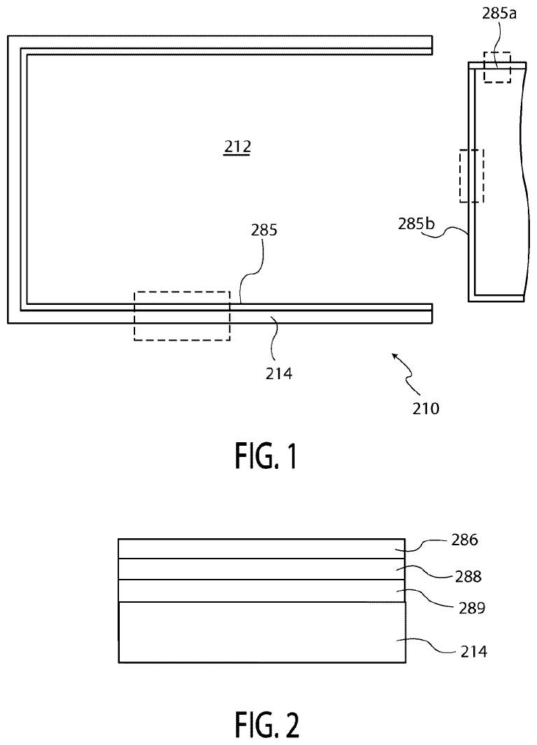

Every embodiment, illustrated most broadly by FIGS. 1 and 2, is a vessel 210 including a wall 214 enclosing a lumen 212 and a vessel coating or layer set 285 on at least a portion of the wall 214 facing the lumen 212. FIG. 1 shows a vessel having at least a single opening; vessels having two openings or more than two openings are also contemplated in any embodiment.

The vessel 210 in any embodiment may also include a closure as shown generically in FIG. 1. Several examples of the closure of FIG. 1 are a stopper, septum, or the like.

The vessel in any embodiment is made of a thermoplastic material, for example cyclic olefin polymer (COP) or cyclic olefin copolymer (COC).

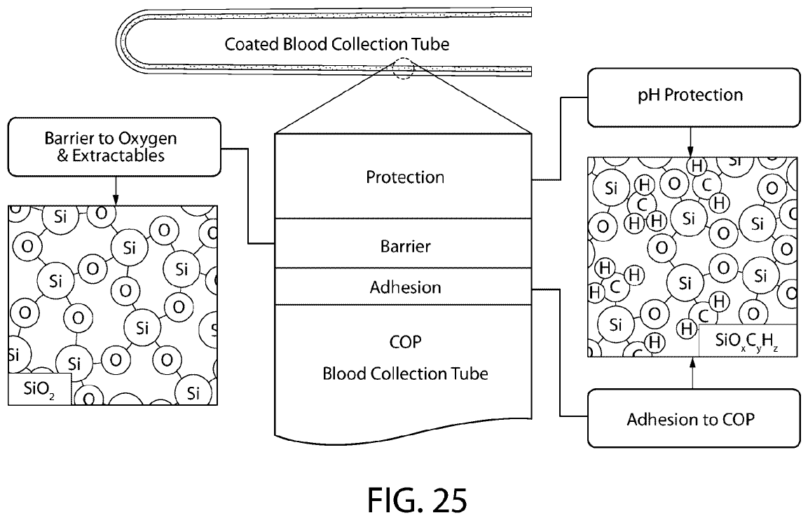

An embodiment of the vessel coating or layer set 285, 285a, or 285b in any embodiment is at least one tie coating or layer 289, at least one barrier coating or layer 288, and at least one pH protective coating or layer 286, illustrated in FIGS. 1-2 and 25 and present in any embodiment. This vessel coating or layer set is sometimes known as a “trilayer coating” in which the barrier coating or layer 288 of SiOₓ is protected against contents having a pH otherwise high enough to remove it by being sandwiched between the pH protective coating or layer 286 and the tie coating or layer 289, each an organic layer of SiOₓC_{y} as defined in this specification. Specific examples of this trilayer coating in any embodiment are provided in this specification. The contemplated thicknesses of the respective layers in nm (preferred ranges in parentheses) are given in the Trilayer Thickness Table.

Trilayer Thickness Table

| Adhesion | Barrier | Protection |
|---|---|---|
| 5-100 | 20-200 | 50-500 |
| (5-20) | (20-30) | (100-200) |

The trilayer coating set 285 includes as a first layer an adhesion or tie coating or layer 289 that improves adhesion of the barrier coating or layer to the COP substrate. The adhesion or tie coating or layer 289 is also believed to relieve stress on the barrier coating or layer 288, making the barrier layer less subject to damage from thermal expansion or contraction or mechanical shock. The adhesion or tie coating or layer 289 is also believed to decouple defects between the barrier coating or layer 288 and the COP substrate. This is believed to occur because any pinholes or other defects that may be formed when the adhesion or tie coating or layer 289 is applied tend not to be continued when the barrier coating or layer 288 is applied, so the pinholes or other defects in one coating do not line up with defects in the other. The adhesion or tie coating or layer 289 has some efficacy as a barrier layer, so even a defect providing a leakage path extending through the barrier coating or layer 289 is blocked by the adhesion or tie coating or layer 289.

The trilayer coating set 285 includes as a second layer a barrier coating or layer 288 that provides a barrier to oxygen that has permeated the COP wall. The barrier coating or layer 288 also is a barrier to extraction of the composition of the vessel wall 214 by the contents of the lumen 214.

The trilayer coating set 285 includes as a third layer a pH protective coating or layer 286 that provides protection of the underlying barrier coating or layer 288 against contents of the vessel, including where a surfactant is present.

The features of each layer of the trilayer coating set are further described below.

### Tie Coating or Layer

The tie coating or layer 289 has at least two functions. One function of the tie coating or layer 289 is to improve adhesion of a barrier coating or layer 288 to a substrate, in particular a thermoplastic substrate. For example, a tie coating or layer, also referred to as an adhesion layer or coating can be applied to the substrate and the barrier layer can be applied to the adhesion layer to improve adhesion of the barrier layer or coating to the substrate.

Another function of the tie coating or layer 289 has been discovered: a tie coating or layer 289 applied under a barrier coating or layer 288 can improve the function of a pH protective coating or layer 286 applied over the barrier coating or layer 288.

The tie coating or layer 289 can be composed of, comprise, or consist essentially of SiOₓC_{y}, in which x is between 0.5 and 2.4 and y is between 0.6 and 3. Alternatively, the atomic ratio can be expressed as the formula Si_{w}OₓC_{y}, The atomic ratios of Si, O, and C in the tie coating or layer 289 are, as several options:
Si 100: O 50-150: C 90-200 (i.e. w=1, x=0.5 to 1.5, y=0.9 to 2);
Si 100: O 70-130: C 90-200 (i.e. w=1, x=0.7 to 1.3, y=0.9 to 2)
Si 100: O 80-120: C 90-150 (i.e. w=1, x=0.8 to 1.2, y=0.9 to 1.5)
Si 100: O 90-120: C 90-140 (i.e. w=1, x=0.9 to 1.2, y=0.9 to 1.4), or
Si 100: O 92-107: C 116-133 (i.e. w=1, x=0.92 to 1.07, y=1.16 to 1.33)

The atomic ratio can be determined by XPS. Taking into account the H atoms, which are not measured by XPS, the tie coating or layer 289 may thus in one aspect have the formula Si_{w}OₓC_{y}H_{z} (or its equivalent SiOₓC_{y}), for example where w is 1, x is from about 0.5 to about 2.4, y is from about 0.6 to about 3, and z is from about 2 to about 9. Typically, tie coating or layer 289 would hence contain 36% to 41% carbon normalized to 100% carbon plus oxygen plus silicon.

Optionally, the tie coating or layer can be similar or identical in composition with the pH protective coating or layer 286 described elsewhere in this specification, although this is not a requirement.

The tie coating or layer 289 is contemplated generally to be from 5 nm to 100 nm thick, preferably from 5 to 20 nm thick, particularly if applied by chemical vapor deposition. These thicknesses are not critical. Commonly but not necessarily, the tie coating or layer 289 will be relatively thin, since its function is to change the surface properties of the substrate.

### Barrier Layer

A barrier coating or layer 288 optionally can be deposited by plasma-enhanced chemical vapor deposition (PECVD) or other chemical vapor deposition processes on the vessel of a pharmaceutical package, in particular, a thermoplastic package to prevent oxygen, carbon dioxide, or other gases from entering the vessel and/or to prevent leaching of the pharmaceutical material into or through the package wall.

The barrier coating or layer for any embodiment defined in this specification (unless otherwise specified in a particular instance) is a coating or layer, optionally applied by PECVD as indicated in U.S. Pat. No. 7,985,188. The barrier layer optionally is characterized as a “SiOₓ” coating, and contains silicon, oxygen, and optionally other elements, in which x, the ratio of oxygen to silicon atoms, is from about 1.5 to about 2.9, or 1.5 to about 2.6, or about 2. These alternative definitions of x apply to any use of the term SiOₓ in this specification. The barrier coating or layer is applied, for example, to the interior of a pharmaceutical package or another vessel, for example, a sample collection tube or another type of vessel.

The barrier coating 288 comprises or consists essentially of SiOₓ, wherein x is from 1.5 to 2.9, from 2 to 1000 nm thick, the barrier coating 288 of SiOₓ having an interior surface 220 facing the lumen 212 and an outer surface 222 facing the wall 214 article surface 254, the barrier coating 288 being effective to reduce the ingress of atmospheric gas into the lumen 212 compared to an uncoated vessel 250. One suitable barrier composition is one where x is 2.3, for example. For example, the barrier coating or layer such as 288 of any embodiment can be applied at a thickness of at least 2 nm, or at least 4 nm, or at least 7 nm, or at least 10 nm, or at least 20 nm, or at least 30 nm, or at least 40 nm, or at least 50 nm, or at least 100 nm, or at least 150 nm, or at least 200 nm, or at least 300 nm, or at least 400 nm, or at least 500 nm, or at least 600 nm, or at least 700 nm, or at least 800 nm, or at least 900 nm. The barrier coating or layer can be up to 1000 nm, or at most 900 nm, or at most 800 nm, or at most 700 nm, or at most 600 nm, or at most 500 nm, or at most 400 nm, or at most 300 nm, or at most 200 nm, or at most 100 nm, or at most 90 nm, or at most 80 nm, or at most 70 nm, or at most 60 nm, or at most 50 nm, or at most 40 nm, or at most 30 nm, or at most 20 nm, or at most 10 nm, or at most 5 nm thick. Ranges of 20-200 nm, optionally 20-30 nm, are contemplated. Specific thickness ranges composed of any one of the minimum thicknesses expressed above, plus any equal or greater one of the maximum thicknesses expressed above, are expressly contemplated.

The thickness of the SiOₓ or other barrier coating or layer can be measured, for example, by transmission electron microscopy (TEM), and its composition can be measured by X-ray photoelectron spectroscopy (XPS). The pH protective coating or layer described herein can be applied to a variety of pharmaceutical packages or other vessels made from plastic or glass, for example, to plastic tubes, vessels, and syringes.

A barrier coating or layer 286 of SiOₓ, in which x is between 1.5 and 2.9, is applied by plasma-enhanced chemical vapor deposition (PECVD) directly or indirectly to the thermoplastic wall 214 (e.g., a tie coating or layer 289 can be interposed between them) so that in the filled pharmaceutical package or other vessel 210 the barrier coating or layer 286 is located between the inner or interior surface 220 of the thermoplastic wall 214 and the fluid 218.

The barrier coating or layer 286 of SiOₓ is supported by the thermoplastic wall 214. The barrier coating or layer 286 as described elsewhere in this specification, or in U.S. Pat. No. 7,985,188, can be used in any embodiment.

Certain barrier coatings or layers 286 such as SiOₓ as defined here have been found to have the characteristic of being subject to being measurably diminished in barrier improvement factor in less than six months as a result of attack by certain relatively high pH contents of the coated vessel as described elsewhere in this specification, particularly where the barrier coating or layer directly contacts the contents. This issue can be addressed using a pH protective coating or layer, as discussed in this specification.

### pH Protective Coating or Layer

The inventors have found that barrier layers or coatings of SiOₓ are eroded or dissolved by some fluids, for example, aqueous compositions having a pH above about 5. Since coatings applied by chemical vapor deposition can be very thin—tens to hundreds of nanometers thick—even a relatively slow rate of erosion can remove or reduce the effectiveness of the barrier layer in less time than the desired shelf life of a product package. This is particularly a problem for fluid pharmaceutical compositions, since many of them have a pH of roughly 7, or more broadly in the range of 5 to 9, similar to the pH of blood and other human or animal fluids. The higher the pH of the pharmaceutical preparation, the more quickly it erodes or dissolves the SiOₓ coating. Optionally, this problem can be addressed by protecting the barrier coating or layer 288, or other pH-sensitive material, with a pH protective coating or layer 286.

Optionally, the pH protective coating or layer 286 can be composed of, comprise, or consist essentially of Si_{W}OₓC_{y}H_{z} (or its equivalent SiOₓC_{y}) or Si_{w}NₓC_{y}H_{z} or its equivalent SiNₓC_{y}), each as defined previously. The atomic ratio of Si:O:C or Si:N:C can be determined by XPS (X-ray photoelectron spectroscopy). Taking into account the H atoms, the pH protective coating or layer may thus in one aspect have the formula Si_{W}OₓC_{y}H, or its equivalent SiOₓC_{y}, for example where w is 1, x is from about 0.5 to about 2.4, y is from about 0.6 to about 3, and z is from about 2 to about 9.

Typically, expressed as the formula Si_{w}OₓC_{y}, the atomic ratios of Si, O, and C are, as several options:
Si 100: O 50-150: C 90-200 (i.e., w=1, x=0.5 to 1.5, y=0.9 to 2);
Si 100: O 70-130: C 90-200 (i.e., w=1, x=0.7 to 1.3, y=0.9 to 2)
Si 100: O 80-120: C 90-150 (i.e., w=1, x=0.8 to 1.2, y=0.9 to 1.5)
Si 100: O 90-120: C 90-140 (i.e., w=1, x=0.9 to 1.2, y=0.9 to 1.4)
Si 100: O 92-107: C 116-133 (i.e., w=1, x=0.92 to 1.07, y=1.16 to 1.33), or
Si 100: O 80-130: C 90-150.

Alternatively, the pH protective coating or layer can have atomic concentrations normalized to 100% carbon, oxygen, and silicon, as determined by X-ray photoelectron spectroscopy (XPS) of less than 50% carbon and more than 25% silicon. Alternatively, the atomic concentrations are from 25 to 45% carbon, 25 to 65% silicon, and 10 to 35% oxygen. Alternatively, the atomic concentrations are from 30 to 40% carbon, 32 to 52% silicon, and 20 to 27% oxygen. Alternatively, the atomic concentrations are from 33 to 37% carbon, 37 to 47% silicon, and 22 to 26% oxygen.

The thickness of the pH protective coating or layer can be, for example:
from 10 nm to 1000 nm;
alternatively from 10 nm to 1000 nm;
alternatively from 10 nm to 900 nm;
alternatively from 10 nm to 800 nm;
alternatively from 10 nm to 700 nm;
alternatively from 10 nm to 600 nm;
alternatively from 10 nm to 500 nm;
alternatively from 10 nm to 400 nm;
alternatively from 10 nm to 300 nm;
alternatively from 10 nm to 200 nm;
alternatively from 10 nm to 100 nm;
alternatively from 10 nm to 50 nm;
alternatively from 20 nm to 1000 nm;
alternatively from 50 nm to 1000 nm;
alternatively from 10 nm to 1000 nm;
alternatively from 50 nm to 800 nm;
alternatively from 100 nm to 700 nm;
alternatively from 300 to 600 nm.

Optionally, the atomic concentration of carbon in the protective layer, normalized to 100% of carbon, oxygen, and silicon, as determined by X-ray photoelectron spectroscopy (XPS), can be greater than the atomic concentration of carbon in the atomic formula for the organosilicon precursor. For example, embodiments are contemplated in which the atomic concentration of carbon increases by from 1 to 80 atomic percent, alternatively from 10 to 70 atomic percent, alternatively from 20 to 60 atomic percent, alternatively from 30 to 50 atomic percent, alternatively from 35 to 45 atomic percent, alternatively from 37 to 41 atomic percent.

Optionally, the atomic ratio of carbon to oxygen in the pH protective coating or layer can be increased in comparison to the organosilicon precursor, and/or the atomic ratio of oxygen to silicon can be decreased in comparison to the organosilicon precursor.

Optionally, the pH protective coating or layer can have an atomic concentration of silicon, normalized to 100% of carbon, oxygen, and silicon, as determined by X-ray photoelectron spectroscopy (XPS), less than the atomic concentration of silicon in the atomic formula for the feed gas. For example, embodiments are contemplated in which the atomic concentration of silicon decreases by from 1 to 80 atomic percent, alternatively by from 10 to 70 atomic percent, alternatively by from 20 to 60 atomic percent, alternatively by from 30 to 55 atomic percent, alternatively by from 40 to 50 atomic percent, alternatively by from 42 to 46 atomic percent.

As another option, a pH protective coating or layer is contemplated that can be characterized by a sum formula wherein the atomic ratio C:O can be increased and/or the atomic ratio Si:O can be decreased in comparison to the sum formula of the organosilicon precursor.

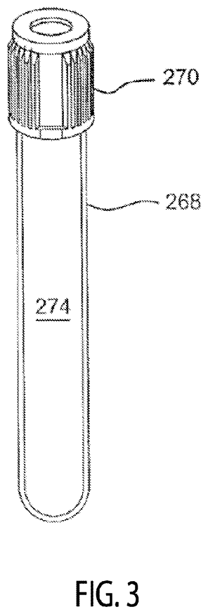

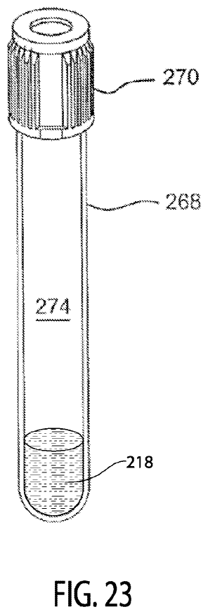

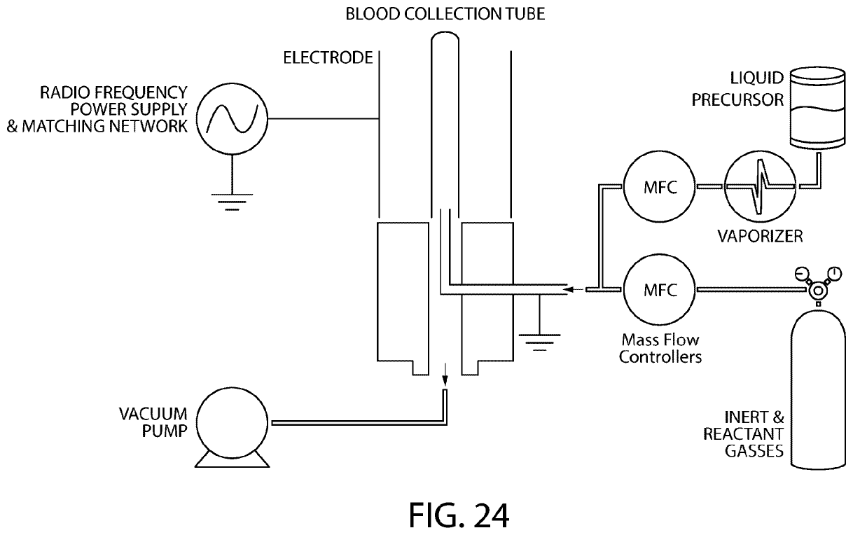

The pH protective coating or layer 286 is commonly located between the barrier coating or layer 288 and the fluid 218 in the finished article such as the pharmaceutical package 210 shown in FIG. 23. In this instance, the pharmaceutical package 210 is a blood sample collection tube or another vessel 268, shown for example in FIG. 3, containing a reagent or other fluid 218 as shown in FIG. 23, and evacuated to facilitate its use for collecting an intravenous blood sample. One non-limiting example of a reagent is an aqueous sodium citrate reagent, which is suitable for preventing or reducing blood coagulation. The pH protective coating or layer 286 is supported by the thermoplastic wall 214.

The pH protective coating or layer 286 optionally is effective to keep the barrier coating or layer 288 at least substantially undissolved as a result of attack by the fluid 218 for a period of at least six months.

The pH protective coating or layer can have a density between 1.25 and 1.65 g/cm³, alternatively between 1.35 and 1.55 g/cm³, alternatively between 1.4 and 1.5 g/cm³, alternatively between 1.4 and 1.5 g/cm³, alternatively between 1.44 and 1.48 g/cm³, as determined by X-ray reflectivity (XRR). Optionally, the organosilicon compound can be octamethylcyclotetrasiloxane and the pH protective coating or layer can have a density that can be higher than the density of a pH protective coating or layer made from HMDSO as the organosilicon compound under the same PECVD reaction conditions.

The pH protective coating or layer optionally can prevent or reduce the precipitation of a compound or component of a composition in contact with the pH protective coating or layer, in particular, can prevent or reduce insulin precipitation or blood clotting, in comparison to the uncoated surface and/or to a barrier coated surface using HMDSO as precursor.

The interior surface of the pH protective coating or layer optionally can have a contact angle (with distilled water) of from 90° to 110°, optionally from 80° to 120°, optionally from 700 to 130°, as measured by Goniometer Angle measurement of a water droplet on the pH protective surface, per ASTM D7334-08 “Standard Practice for Surface Wettability of Coatings, Substrates and Pigments by Advancing Contact Angle Measurement.”

The passivation layer or pH protective coating or layer 286 optionally shows an O-Parameter measured with attenuated total reflection (ATR) of less than 0.4, measured as:

$$O - Parameter = \frac{Intensity at 1253 cm - 1}{Maximum intensity in the range 1000 to 1100 cm - 1}$$

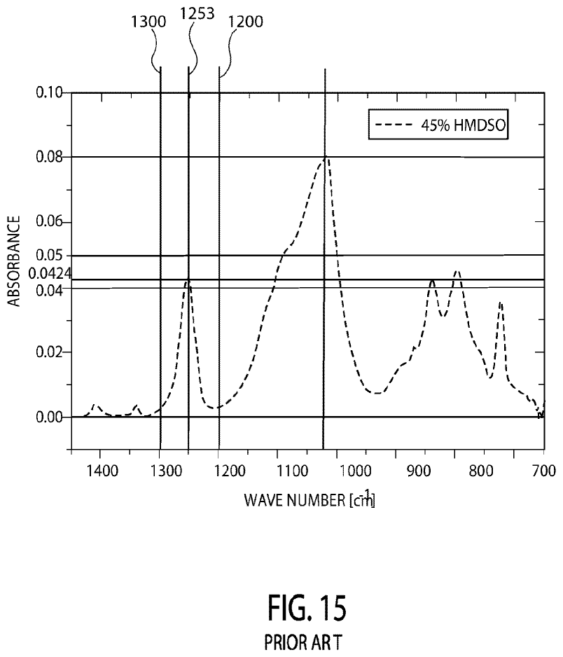

The O-Parameter is defined in U.S. Pat. No. 8,067,070, which claims an O-parameter value of most broadly from 0.4 to 0.9. It can be measured from physical analysis of an FTIR amplitude versus wave number plot to find the numerator and denominator of the above expression, as shown in FIG. 15, which is the same as FIG. 5 of U.S. Pat. No. 8,067,070, except annotated to show interpolation of the wave number and absorbance scales to arrive at an absorbance at 1253 cm⁻¹ of 0.0424 and a maximum absorbance at 1000 to 1100 cm⁻¹ of 0.08, resulting in a calculated O-parameter of 0.53. The O-Parameter can also be measured from digital wave number versus absorbance data.

U.S. Pat. No. 8,067,070 asserts that the claimed O-parameter range provides a superior pH protective coating or layer, relying on experiments only with HMDSO and HMDSN, which are both non-cyclic siloxanes. Surprisingly, it has been found by the present inventors that if the PECVD precursor is a cyclic siloxane, for example OMCTS, O-parameters outside the ranges claimed in U.S. Pat. No. 8,067,070, using OMCTS, provide even better results than are obtained in U.S. Pat. No. 8,067,070 with HMDSO.

Alternatively, in any embodiment, the O-parameter has a value of from 0.1 to 0.39, or from 0.15 to 0.37, or from 0.17 to 0.35.

Even another aspect is a composite material according to any embodiment, wherein the passivation layer shows an N-Parameter measured with attenuated total reflection (ATR) of less than 0.7, measured as:

$$N - Parameter = \frac{Intensity at 840 {cm}^{- 1}}{Intensity at 799 {cm}^{- 1}} .$$

The N-Parameter is also described in U.S. Pat. No. 8,067,070, and is measured analogously to the O-Parameter except that intensities at two specific wave numbers are used—neither of these wave numbers is a range. U.S. Pat. No. 8,067,070 claims a passivation layer with an N-Parameter of 0.7 to 1.6. Again, the present inventors have made better coatings employing a pH protective coating or layer 286 having an N-Parameter lower than 0.7, as described above. Alternatively, the N-parameter has a value of at least 0.3, or from 0.4 to 0.6, or at least 0.53.

The rate of erosion, dissolution, or leaching (different names for related concepts) of the pH protective coating or layer 286, if directly contacted by the fluid 218, is less than the rate of erosion of the barrier coating or layer 288, if directly contacted by the fluid 218.

The thickness of the pH protective coating or layer is contemplated to be from 50-500 nm, with a preferred range of 100-200 nm.

The pH protective coating or layer 286 is effective to isolate the fluid 218 from the barrier coating or layer 288, at least for sufficient time to allow the barrier coating to act as a barrier during the shelf life of the pharmaceutical package or another vessel 210.

The inventors have further found that certain pH protective coatings or layers of SiOₓC_{y} or SiNₓC_{y} formed from cyclic polysiloxane precursors, which pH protective coatings or layers have a substantial organic component, do not erode quickly when exposed to fluids, and in fact erode or dissolve more slowly when the fluids have higher pHs within the range of 5 to 9. For example, at pH 8, the dissolution rate of a pH protective coating or layer made from the precursor octamethylcyclotetrasiloxane, or OMCTS, is quite slow. These pH protective coatings or layers of SiOₓC_{y} or SiNₓC_{y} can therefore be used to cover a barrier layer of SiOₓ, retaining the benefits of the barrier layer by protecting it from the fluid in the pharmaceutical package. The protective layer is applied over at least a portion of the SiOₓ layer to protect the SiOₓ layer from contents stored in a vessel, where the contents otherwise would be in contact with the SiOₓ layer.

Although the present disclosure does not depend upon the accuracy of the following theory, it is further believed that effective pH protective coatings or layers for avoiding erosion can be made from cyclic siloxanes and silazanes as described in this disclosure. SiOₓC_{y} or SiNₓC_{y} coatings deposited from cyclic siloxane or linear silazane precursors, for example, octamethylcyclotetrasiloxane (OMCTS), are believed to include intact cyclic siloxane rings and longer series of repeating units of the precursor structure. These coatings are believed to be nanoporous but structured and hydrophobic, and these properties are believed to contribute to their success as pH protective coatings or layers, and also protective coatings or layers. This is shown, for example, in U.S. Pat. No. 7,901,783.

SiOₓC_{y} or SiNₓC_{y} coatings also can be deposited from linear siloxane or linear silazane precursors, for example, hexamethyldisiloxane (HMDSO) or tetramethyldisiloxane (TMDSO).

Optionally an FTIR absorbance spectrum of the pH protective coating or layer 286 of any embodiment has a ratio greater than 0.75 between the maximum amplitude of the Si—O—Si symmetrical stretch peak normally located between about 1000 and 1040 cm−1, and the maximum amplitude of the Si—O—Si asymmetric stretch peak normally located between about 1060 and about 1100 cm−1. Alternatively in any embodiment, this ratio can be at least 0.8, or at least 0.9, or at least 1.0, or at least 1.1, or at least 1.2. Alternatively in any embodiment, this ratio can be at most 1.7, or at most 1.6, or at most 1.5, or at most 1.4, or at most 1.3. Any minimum ratio stated here can be combined with any maximum ratio stated here, as an alternative embodiment.

Optionally, in any embodiment, the pH protective coating or layer 286, in the absence of the medicament, has a non-oily appearance. This appearance has been observed in some instances to distinguish an effective pH protective coating or layer from a lubricity layer, which in some instances has been observed to have an oily (i.e., shiny) appearance.

Optionally, for the pH protective coating or layer 286 in any embodiment, the silicon dissolution rate by a 50 mM potassium phosphate buffer diluted in water for injection, adjusted to pH 8 with concentrated nitric acid, and containing 0.2 wt. % polysorbate-80 surfactant, (measured in the absence of the medicament, to avoid changing the dissolution reagent), at 40° C., is less than 170 ppb/day. (Polysorbate-80 is a common ingredient of pharmaceutical preparations, available for example as Tween®-80 from Uniqema Americas LLC, Wilmington Delaware.)

Optionally, for the pH protective coating or layer 286 in any embodiment, the silicon dissolution rate is less than 160 ppb/day, or less than 140 ppb/day, or less than 120 ppb/day, or less than 100 ppb/day, or less than 90 ppb/day, or less than 80 ppb/day. Optionally, in any embodiment the silicon dissolution rate is more than 10 ppb/day, or more than 20 ppb/day, or more than 30 ppb/day, or more than 40 ppb/day, or more than 50 ppb/day, or more than 60 ppb/day. Any minimum rate stated here can be combined with any maximum rate stated here for the pH protective coating or layer 286 in any embodiment.

Optionally, for the pH protective coating or layer 286 in any embodiment the total silicon content of the pH protective coating or layer and barrier coating, upon dissolution into a test composition with a pH of 8 from the vessel, is less than 66 ppm, or less than 60 ppm, or less than 50 ppm, or less than 40 ppm, or less than 30 ppm, or less than 20 ppm.

### pH Protective Coating Or Layer Properties Of Any Embodiment

### Theory of Operation

The inventors offer the following theory of operation of the pH protective coating or layer described here. The disclosure is not limited by the accuracy of this theory or to the embodiments predictable by use of this theory.

The dissolution rate of the SiOₓ barrier layer is believed to be dependent on SiO bonding within the layer. Oxygen bonding sites (silanols) are believed to increase the dissolution rate.

It is believed that the OMCTS-based pH protective coating or layer bonds with the silanol sites on the SiOₓ barrier layer to “heal” or passivate the SiOₓ surface and thus dramatically reduces the dissolution rate. In this hypothesis, the thickness of the OMCTS layer is not the primary means of protection—the primary means is passivation of the SiOₓ surface. It is contemplated that a pH protective coating or layer, as described in this specification, can be improved by increasing the crosslink density of the pH protective coating or layer.

Use of a coating or layer according to any described embodiment is contemplated as a pH protective coating or layer preventing dissolution of the barrier coating in contact with a fluid.

### EXAMPLES

### Example 1: Conditions for Production of pH Protective Layer

Some conditions optionally used for production of pH Protective Layers are shown in Table 1.

TABLE 1
OMCTS-BASED PLASMA PH PROTECTIVE COATING
OR LAYER MADE WITH CARRIER GAS

| | | | pH | | | | pH protective |
|---|---|---|---|---|---|---|---|
| | pH | | protective | protective | protective | Carrier | coating or |
| | protective | PH | coating or | OMCTS | O2 Flow | Gas (Ar) | layer |
| Example | coating or | protective | layer | Flow Rate | Rate | Flow Rate | Power |
| 1 run | layer Type | Monomer | Time (sec) | (sccm) | (sccm) | (sccm) | (Watts) |
| A | Uncoated | n/a | n/a | n/a | n/a | n/a | n/a |
| (Control) | COC | | | | | | |
| B | Silicon oil | n/a | n/a | n/a | n/a | n/a | n/a |
| (Industry | on COC | | | | | | |
| Standard) | | | | | | | |
| C | L3 lubricity | OMCTS | 10 sec | 3 | 0 | 65 | 6 |
| (without | coating or | | | | | | |
| Oxygen) | layer over | | | | | | |
| | SiOₓ on | | | | | | |
| | COC | | | | | | |
| D | L2 pH | OMCTS | 10 sec | 3 | 1 | 65 | 6 |
| (with | protective | | | | | | |
| Oxygen) | coating or | | | | | | |
| | layer over | | | | | | |
| | SiOₓ on | | | | | | |
| | COC | | | | | | |

### Examples 2-5

Vessel samples were produced as follows. A COC 8007 vessel was produced according to the Protocol for Forming COC Vessel. An SiOₓ barrier coating or layer was applied to the vessels according to the Protocol for Coating COC Vessel Interior with SiOₓ. A pH protective coating or layer was applied to the SiOₓ coated vessels according to the Protocol for Coating COC Vessel Interior with OMCTS, modified as follows. Argon carrier gas and oxygen were used where noted in Table 2.

The process conditions were set to the following, or as indicated in Table 2:
OMCTS—3 sccm (when used)
Argon gas—7.8 sccm (when used)
Oxygen 0.38 sccm (when used)
Power—3 watts
Power on time—10 seconds
   Vessels 2, 3, and 4 of the corresponding example numbers were tested to determine total extractable silicon levels (representing extraction of the organosilicon-based PECVD pH protective coating or layer) using the Protocol for Measuring Dissolved Silicon in a Vessel, modified and supplemented as shown in this example.

The silicon was extracted using saline water digestion. The vessel was filled with two milliliters of 0.9% aqueous saline solution and sealed. The vessel was set into a PTFE test stand and placed in an oven at 50° C. for 72 hours.

Then, the saline solution was removed from the vessel and the fluid obtained from each vessel was brought to a volume of 50 ml using 18.2MΩ-cm deionized water and further diluted 2× to minimize sodium background during analysis.

Next, the fluid recovered from each vessel was tested for extractable silicon using the Protocol for Measuring Dissolved Silicon in a Vessel. The instrument used was a Perkin Elmer Elan DRC II equipped with a Cetac ASX-520 autosampler. The following ICP-MS conditions were employed:
Nebulizer: Quartz Meinhardt
Spray Chamber: Cyclonic
RF (radio frequency) power: 1550 Watts
Argon (Ar) Flow: 15.0 L/min
Auxillary Ar Flow: 1.2 L/min
Nebulizer Gas Flow: 0.88 L/min
Integration time: 80 sec
Scanning mode: Peak hopping
RPq (The RPq is a rejection parameter) for Cerium as CeO (m/z 156: <2%

Aliquots from aqueous dilutions obtained from vessels 2, 3, and 4 were injected and analyzed for Si in concentration units of micrograms per liter. The results of this test are shown in Table 2. While the results are not quantitative, they do indicate that extractables from the pH protective coating or layer are not clearly higher than the extractables for the SiOₓ barrier layer only.

TABLE 2
OMCTS PH PROTECTIVE COATING
OR LAYER ((Ex. 2 and 3))

| Example | OMCTS | O₂ | Ar |
|---|---|---|---|
| (Vessel) | (sccm) | (sccm) | (sccm) |
| 2 | 3.0 | 0.38 | 7.8 |
| 3 | 3.0 | 0.38 | 7.8 |
| 4 | n/a | n/a | n/a |
| (SiOₓonly) | | | |
| 5 | n/a | n/a | n/a |
| (silicon oil) | | | |

### Examples 6-8

Vessel samples 6, 7, and 8, employing three different pH protective coatings or layers, were produced in the same manner as for Examples 2-5 except as otherwise indicated in Table 3:

Vessel 6 had a three-component pH protective coating or layer employing OMCTS, oxygen, and carrier gas. Vessel 7 had a two-component pH protective coating or layer employing OMCTS and oxygen, but no carrier gas. Vessel 8 had a one-component pH protective coating or layer (OMCTS only). Vessels 6-8 were then tested for lubricity as described for Examples 2-5.

The pH protective coatings or layers produced according to these working examples are also contemplated to function as protective coatings or layers to increase the shelf life of the vessels, compared to similar vessels provided with a barrier coating or layer but no pH protective coating or layer.

TABLE 3
OMCTS pH PROTECTIVE COATING OR LAYER

OMCTS - 2.5 sccm
Argon gas - 7.6 sccm (when used)
Oxygen 0.38 sccm (when used)
Power - 3 watts
Power on time - 10 seconds

### Examples 9-11

Examples 6-8 using an OMCTS precursor gas were repeated in Examples 9-11, except that HMDSO was used as the precursor in Examples 9-11. The results are shown in Table 4. The coatings produced according to these working examples are contemplated to function as pH protective coatings or layers, and also as protective coatings or layers to increase the shelf life of the vessels, compared to similar vessels provided with a barrier coating or layer but no pH protective coating or layer.

TABLE 4
HMDSO pH PROTECTIVE COATING OR LAYER

| | HMDSO | O₂ | Ar |
|---|---|---|---|
| Example | (sccm) | (sccm) | (sccm) |
| 9 | 2.5 | 0.38 | 7.6 |
| 10 | 2.5 | 0.38 | — |
| 11 | 2.5 | — | — |

### Example 12: PH Protective Coating or Layer Extractables

Silicon extractables from vessels were measured using ICP-MS analysis as described in the Protocol for Measuring Dissolved Silicon in a Vessel. The vessels were evaluated in both static and dynamic situations. The Protocol for Measuring Dissolved Silicon in a Vessel, modified as follows, describes the test procedure:
Vessel filled with 2 ml of 0.9% saline solution
Vessel placed in a stand—stored at 50° C. for 72 hours.
After 72 hours saline solution test for dissolved silicon
Dissolved silicon measured before and after saline solution expelled from vessel.

The extractable Silicon Levels from a silicon oil-coated glass vessel and a protective coated and SiOₓ coated COC vessel are shown in Table 5. The precision of the ICP-MS total silicon measurement is +/−3%.

TABLE 5
Silicon Extractables Comparison of SiOₓC_{y}H_{z} Coatings

| Vessel Type | Static (ug/L) | Dynamic (ug/L) |
|---|---|---|
| Cyclic olefin vessel with | 70 | 81 |
| SiOₓC_{y}H_{z} coating | | |
| Borosilicate glass vessel | 825 | 835 |
| with silicone oil | | |

Summary of Lubricity and/or Protective Measurements

Table 6 shows a summary of the above OMCTS coatings or layers

TABLE 6
Summary Table of OMCTS PH PROTECTIVE COATING OR LAYER
From Selected Previous Examples

| | OMCTS | O₂ | Ar | Power | Dep Time |
|---|---|---|---|---|---|
| Example | (sccm) | (sccm) | (sccm) | (Watt) | (sec) |
| 1C | 3.0 | 0.00 | 65 | 6 | 10 |
| 1D | 3.0 | 1.00 | 65 | 6 | 10 |
| 2 | 3.0 | 0.38 | 7.8 | 6 | 10 |
| 3 | 3.0 | 0.38 | 7.8 | 6 | 10 |
| 6 | 2.5 | 0.38 | 7.6 | 6 | 10 |
| 7 | 2.5 | 0.38 | 0.0 | 6 | 10 |
| 8 | 2.5 | 0.00 | 0.0 | 6 | 10 |

### Comparative Example 13: Dissolution of SiO: Coating Versus pH

The Protocol for Measuring Dissolved Silicon in a Vessel is followed, except as modified here. Test solutions—50 mM buffer solutions at pH 3, 6, 7, 8, 9, and 12 are prepared. Buffers are selected having appropriate pKa values to provide the pH values being studied. A potassium phosphate buffer is selected for pH 3, 7, 8 and 12, a sodium citrate buffer is utilized for pH 6 and tris buffer is selected for pH 9. 3 ml of each test solution is placed in borosilicate glass 5 ml pharmaceutical vessels and SiOₓ coated 5 ml thermoplastic pharmaceutical vessels. The vessels are all closed with standard coated stoppers and crimped. The vessels are placed in storage at 20-25° C. and pulled at various time points for inductively coupled plasma spectrometer (ICP) analysis of Si content in the solutions contained in the vessels, in parts per billion (ppb) by weight, for different storage times.

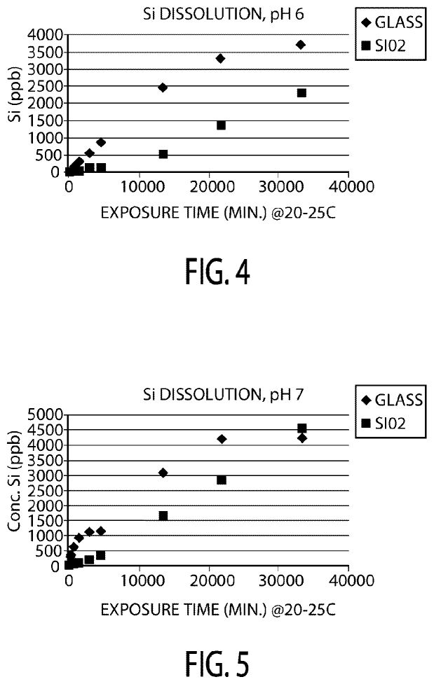

The Protocol for Determining Average Dissolution Rate Si content is used to monitor the rate of glass dissolution, except as modified here. The data is plotted to determine an average rate of dissolution of borosilicate glass or SiOₓ coating at each pH condition. Representative plots at pH 6 through 8 are FIGS. 4-6.

The rate of Si dissolution in ppb is converted to a predicted thickness (nm) rate of Si dissolution by determining the total weight of Si removed, then using a surface area calculation of the amount of vessel surface (11.65 cm²) exposed to the solution and a density of SiOₓ of 2.2 g/cm³. FIG. 7 shows the predicted initial thickness of the SiOₓ coating required, based on the conditions and assumptions of this example (assuming a residual SiOₓ coating of at least 30 nm at the end of the desired shelf life of two years, and assuming storage at 20 to 25° C.). As FIG. 7 shows, the predicted initial thickness of the coating is about 36 nm at pH 5, about 80 nm at pH 6, about 230 nm at pH 7, about 400 nm at pH 7.5, about 750 nm at pH 8, and about 2600 nm at pH 9.

The coating thicknesses in FIG. 7 represent atypically harsh case scenarios for pharma and biotech products. As a general rule of thumb, storage at a lower temperature reduces the thickness required, all other conditions being equivalent.

The following conclusions are reached, based on this test. First, the amount of dissolved Si in the SiOₓ coating or glass increases exponentially with increasing pH. Second, the SiOₓ coating dissolves more slowly than borosilicate glass at a pH lower than 8. The SiOₓ coating shows a linear, monophasic dissolution over time, whereas borosilicate glass tends to show a more rapid dissolution in the early hours of exposure to solutions, followed by a slower linear dissolution. This may be due to surface accumulation of some salts and elements on borosilicate during the forming process relative to the uniform composition of the SiOₓ coating. This result incidentally suggests the utility of an SiOₓ coating on the wall of a borosilicate glass vessel to reduce dissolution of the glass at a pH lower than 8. Third, PECVD applied barrier coatings for vessels in which pharmaceutical preparations are stored will need to be adapted to the specific pharmaceutical preparation and proposed storage conditions (or vice versa), at least in some instances in which the pharmaceutical preparation interacts with the barrier coating significantly.

### Example 14

An experiment is conducted with vessels coated with SiOₓ coating+OMCTS pH protective coating or layer, to test the pH protective coating or layer for its functionality as a protective coating or layer. The vessels are 5 mL vessels (the vessels are normally filled with product to 5 mL; their capacity without headspace, when capped, is about 7.5 mL) composed of cyclic olefin co-polymer (COC, Topas® 6013M-07).

Sixty vessels are coated on their interior surfaces with an SiOₓ coating produced in a plasma-enhanced chemical vapor deposition (PECVD) process using a HMDSO precursor gas according to the Protocol for Coating Tube Interior with SiOₓ set forth above, except that equipment suitable for coating a vessel is used. The following conditions are used.
HMDSO flow rate: 0.47 sccm
Oxygen flow rate: 7.5 sccm
RF power: 70 Watts
Coating time: 12 seconds (includes a 2-sec RF power ramp-up time)

Next the SiOₓ coated vessels are coated over the SiOₓ with an SiOₓC_{y} coating produced in a PECVD process using an OMCTS precursor gas according to the Protocol for Coating COC Vessel Interior with OMCTS Lubricity Coating set forth above, except that the same coating equipment is used as for the SiOₓ coating. The following conditions are used.
OMCTS flow rate: 2.5 sccm
Argon flow rate: 10 sccm
Oxygen flow rate: 0.7 sccm
RF power: 3.4 Watts
Coating time: 5 seconds

Eight vessels are selected and the total deposited quantity of PECVD coating (SiOₓ+SiOₓC_{y}) is determined with a Perkin Elmer Optima Model 7300DV ICP-OES instrument, using the Protocol for Total Silicon Measurement set forth above. This measurement determines the total amount of silicon in both coatings, and does not distinguish between the respective SiOₓ and SiOₓC_{y} coatings. The results are shown below.

| Example, Vessel | Total Silicon ug/L |
|---|---|
| 14-1 | 13844 |
| 14-2 | 14878 |
| 14-3 | 14387 |
| 14-4 | 13731 |
| 14-5 | 15260 |
| 14-6 | 15017 |
| 14-7 | 15118 |
| 14-8 | 12736 |
| Mean | 14371 |
| StdDev | 877 |

Quantity of SiOₓ + Lubricity layer on Vessels

In the following work, except as indicated otherwise in this example, the Protocol for Determining Average Dissolution Rate is followed. Two buffered pH test solutions are used in the remainder of the experiment, respectively at pH 4 and pH 8 to test the effect of pH on dissolution rate. Both test solutions are 50 mM buffers using potassium phosphate as the buffer, diluted in water for injection (WFI) (0.1 um sterilized, filtered). The pH is adjusted to pH 4 or 8, respectively, with concentrated nitric acid.

25 vessels are filled with 7.5 ml per vessel of pH 4 buffered test solution and 25 other vessels are filled with 7.5 ml per vessel of pH 8 buffered test solution (note the fill level is to the top of the vessel—no headspace). The vessels are closed using prewashed butyl stoppers and aluminum crimps. The vessels at each pH are split into two groups. One group at each pH containing 12 vessels is stored at 4° C. and the second group of 13 vessels is stored at 23° C.

The vessels are sampled at Days 1, 3, 6, and 8. The Protocol for Measuring Dissolved Silicon in a Vessel is used, except as otherwise indicated in this example. The analytical result is reported on the basis of parts per billion of silicon in the buffered test solutions of each vessel. A dissolution rate is calculated in terms of parts per billion per day as described above in the Protocol for Determining Average Dissolution Rate. The results at the respective storage temperatures follow:

Shelf Life Conditions 23° C.

| | Vessel SiOₓ + | Vessel SiOₓ + |
|---|---|---|
| | Lubricity | Lubricity |
| | Coating at pH4 | Coating at pH8 |
| Si Dissolution | 31 | 7 |
| Rate (PPB/day) | | |

Shelf Life Conditions 4° C.

| | Vessel SiOₓ + | Vessel SiOₓ + |
|---|---|---|
| | Lubricity | Lubricity |
| | Coating at pH4 | Coating at pH8 |
| Si Dissolution | 7 | 11 |
| Rate (PPB/day) | | |

The observations of Si dissolution versus time for the OMCTS-based coating at pH8 and pH4 indicate the pH4 rates are higher at ambient conditions. Thus, the pH4 rates are used to determine how much material would need to be initially applied to leave a coating of adequate thickness at the end of the shelf life, taking account of the amount of the initial coating that would be dissolved. The results of this calculation are:

| | Vessel SiOₓ + Lubricity |
|---|---|
| | Coating at pH4 |
| Si Dissolution Rate (PPB/day) | 31 |
| Mass of Coating Tested (Total Si) | 14,371 |
| Shelf Life (days) at 23° C. | 464 |
| Shelf Life (years) at 23° C. | 1.3 |
| Required Mass of Coating | 22,630 |
| (Total Si) - 2 years | |
| Required Mass of Coating | 33,945 |
| (Total Si) - 3 years | |

### Shelf Life Calculation

Based on this calculation, the OMCTS protective layer needs to be about 2.5 times thicker—resulting in dissolution of 33945 ppb versus the 14,371 ppb representing the entire mass of coating tested—to achieve a 3-year calculated shelf life.

### Example 15

The results of Comparative Example 13 and Example 14 above can be compared as follows, where the “pH protective coating or layer” is the coating of SiOₓC_{y} referred to in Example BB.

Shelf Life Conditions - - pH8 and 23° C.

| | | Vessel SiOₓ + |
|---|---|---|
| | Vessel SiOₓ | Lubricity Coating |
| Si Dissolution | 1,250 | 7 |
| Rate (PPB/day) | | |

This data shows that the silicon dissolution rate of SiOₓ alone is reduced by more than 2 orders of magnitude at pH 8 in vessels also coated with SiOₓC_{y} coatings.

Another comparison is shown by the following data from several different experiments carried out under similar accelerated dissolution conditions.

| | Silicon Dissolution with pH 8 at 40° C. | | | | | | |
|---|---|---|---|---|---|---|---|
| | (ug/L) | | | | | | |
| Vessel Coating | 1 | 2 | 3 | 4 | 7 | 10 | 15 |
| Description | day | days | days | days | days | days | days |
| A. SiOₓ made | 165 | 211 | 226 | 252 | 435 | 850 | 1,364 |
| with HMDSO | | | | | | | |
| Plasma + | | | | | | | |
| Si_{w}OₓC_{y} or its | | | | | | | |
| equivalent | | | | | | | |
| SiOₓC_{y} made | | | | | | | |
| with OMCTS | | | | | | | |
| Plasma | | | | | | | |
| B. Si_{w}OₓC_{y} or | 109 | 107 | 76 | 69 | 74 | 158 | 198 |
| its equivalent | | | | | | | |
| SiOₓC_{y} made | | | | | | | |
| with OMCTS | | | | | | | |
| Plasma | | | | | | | |
| C. SiOₓ made | 2,504 | 4,228 | 5,226 | 5,650 | 9,292 | 10,177 | 9,551 |
| with HMDSO | | | | | | | |
| Plasma | | | | | | | |
| D. SiOₓ made | 1,607 | 1,341 | 3,927 | 10,182 | 18,148 | 20,446 | 21,889 |
| with HMDSO | | | | | | | |
| Plasma + | | | | | | | |
| Si_{w}OₓC_{y} or its | | | | | | | |
| equivalent | | | | | | | |
| SiOₓC_{y} made | | | | | | | |
| with HMDSO | | | | | | | |
| Plasma | | | | | | | |
| E. Si_{w}OₓC_{y} or | 1,515 | 1,731 | 1,813 | 1,743 | 2,890 | 3,241 | 3,812 |
| its equivalent | | | | | | | |
| SiOₓC_{y} made | | | | | | | |
| with HMDSO | | | | | | | |
| Plasma | | | | | | | |

Row A (SiOₓ with OMCTS coating) versus C (SiOₓ without OMCTS coating) show that the OMCTS pH protective coating or layer is also an effective protective coating or layer to the SiOₓ coating at pH 8. The OMCTS coating reduced the one-day dissolution rate from 2504 ug/L (“u” or μ or the Greek letter “mu” as used herein are identical, and are abbreviations for “micro” ˜) to 165 ug/L.

### Example 16

Samples 1-6 as listed in Table 7 were prepared as described in Example 13, with further details as follows.

A cyclic olefin copolymer (COC) resin was injection molded to form a batch of 5 ml vessels. Silicon chips were adhered with double-sided adhesive tape to the internal walls of the vessels. The vessels and chips were coated with a two-layer coating by plasma-enhanced chemical vapor deposition (PECVD). The first layer was composed of SiOₓ with barrier properties as defined in the present disclosure, and the second layer was an SiOₓC_{y} pH protective coating or layer.

A precursor gas mixture comprising OMCTS, argon, and oxygen was introduced inside each vessel. The gas inside the vessel was excited between capacitively coupled electrodes by a radio-frequency (13.56 MHz) power source. The monomer flow rate (Fm) in units of sccm, oxygen flow rate (Fo) in units of sccm, argon flowrate in sccm, and power (W) in units of watts are shown in Table 7.

A composite parameter, W/FM in units of kJ/kg, was calculated from process parameters W, Fm, Fo and the molecular weight, M in g/mol, of the individual gas species. W/FM is defined as the energy input per unit mass of polymerizing gases. Polymerizing gases are defined as those species that are incorporated into the growing coating such as, but not limited to, the monomer and oxygen. Non-polymerizing gases, by contrast, are those species that are not incorporated into the growing coating, such as but not limited to argon, helium and neon.

In this test, PECVD processing at high W/FM is believed to have resulted in higher monomer fragmentation, producing organosiloxane coatings with higher cross-link density. PECVD processing at low W/FM, by comparison, is believed to have resulted in lower monomer fragmentation producing organosiloxane coatings with a relatively lower cross-link density.

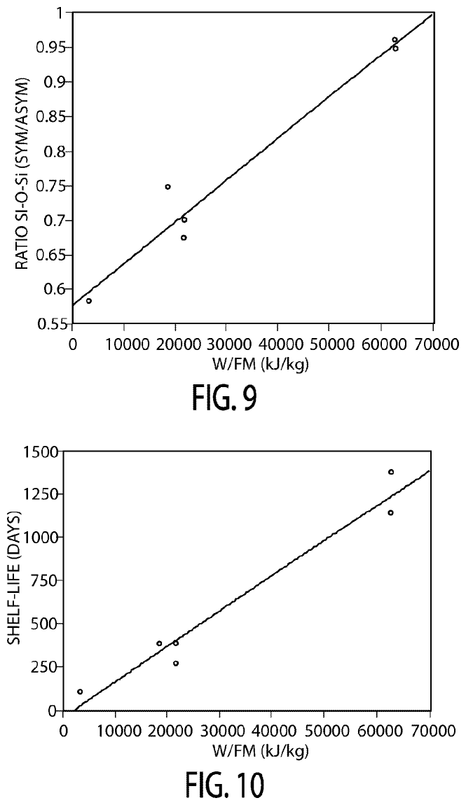

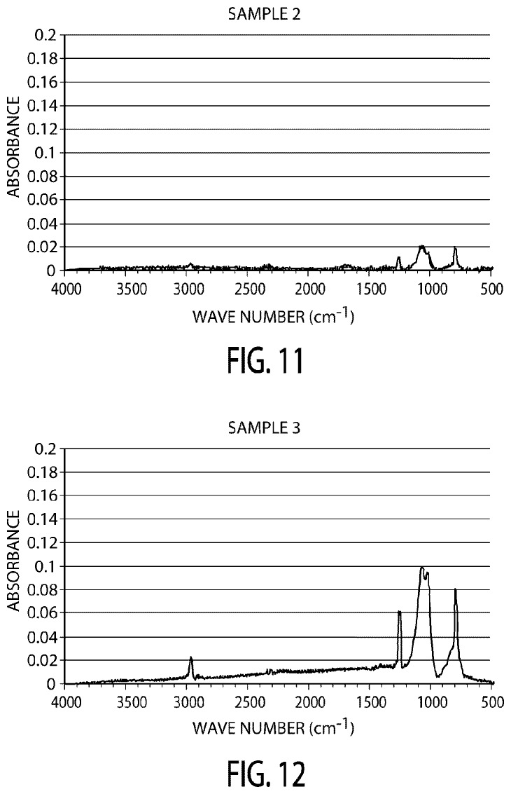

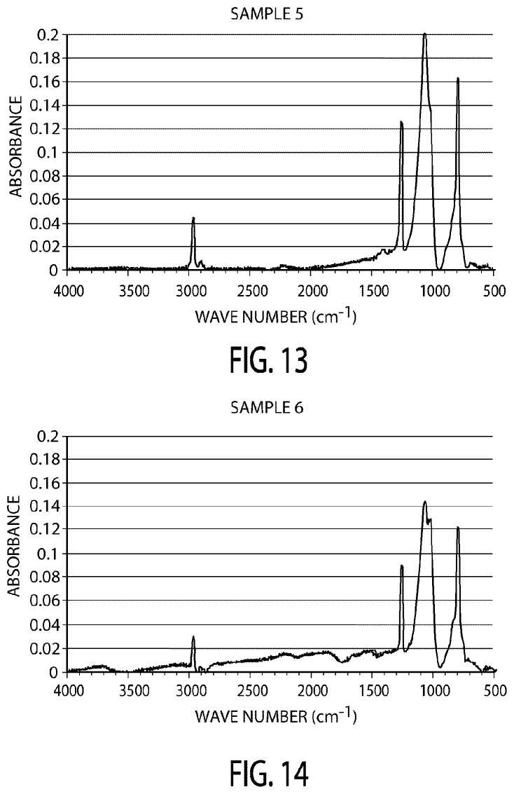

The relative cross-link density of samples 5, 6, 2, and 3 was compared between different coatings by measuring FTIR absorbance spectra. The spectra of samples 5, 6, 2, and 3 are provided in FIGS. 11-14. In each spectrum, the ratio of the peak absorbance at the symmetric stretching mode (1000-1040 cm−1) versus the peak absorbance at the asymmetric stretching mode (1060-1100 cm−1) of the Si—O—Si bond was measured, and the ratio of these two measurements was calculated, all as shown in Table 7. The respective ratios were found to have a linear correlation to the composite parameter W/FM as shown in FIGS. 9-10.

A qualitative relation—whether the coating appeared oily (shiny, often with iridescence) or non-oily (non-shiny) when applied on the silicon chips—was also found to correlate with the W/FM values in Table 7. Oily appearing coatings deposited at lower W/FM values, as confirmed by Table 7, are believed to have a lower crosslink density, as determined by their lower sym/asym ratio, relative to the non-oily coatings that were deposited at higher W/FM and a higher cross-link density. The only exception to this general rule of thumb was sample 2 in Table 7. It is believed that the coating of sample 2 exhibited a non-oily appearance because it was too thin to see. Thus, an oilyness observation was not reported in Table 7 for sample 2. The chips were analyzed by FTIR in transmission mode, with the infrared spectrum transmitted through the chip and sample coating, and the transmission through an uncoated null chip subtracted.

Non-oily organosiloxane layers produced at higher W/FM values, which protect the underlying SiOₓ coating from aqueous solutions at elevated pH and temperature, were preferred because they provided lower Si dissolution and a longer shelf life, as confirmed by Table 7. For example, the calculated silicon dissolution by contents of the vessel at a pH of 8 and 40° C. was reduced for the non-oily coatings, and the resulting shelf life was 1381 days in one case and 1147 days in another, as opposed to the much shorter shelf lives and higher rates of dissolution for oily coatings. Calculated shelf life was determined as shown for Example 13. The calculated shelf life also correlated linearly to the ratio of symmetric to asymmetric stretching modes of the Si—O—Si bond in organosiloxane pH protective coatings or layers.

Sample 6 can be particularly compared to Sample 5. An organosiloxane, pH protective coating or layer was deposited according to the process conditions of sample 6 in Table 7. The coating was deposited at a high W/FM. This resulted in a non-oily coating with a high Si—O—Si sym/asym ratio of 0.958, which resulted in a low rate of dissolution of 84.1 ppb/day (measured by the Protocol for Determining Average Dissolution Rate) and long shelf life of 1147 days (measured by the Protocol for Determining Calculated Shelf Life). The FTIR spectra of this coating exhibits a relatively similar asymmetric Si—O—Si peak absorbance compared to the symmetric Si—O—Si peak absorbance. This is an indication of a higher cross-link density coating, which is a preferred characteristic for pH protection and long shelf life.

An organosiloxane pH protective coating or layer was deposited according to the process conditions of sample 5 in Table 7. The coating was deposited at a moderate W/FM. This resulted in an oily coating with a low Si—O—Si sym/asym ratio of 0.673, which resulted in a high rate of dissolution of 236.7 ppb/day (following the Protocol for Determining Average Dissolution Rate) and shorter shelf life of 271 days (following the Protocol for Determining Calculated Shelf Life). The FTIR spectrum of this coating exhibits a relatively high asymmetric Si—O—Si peak absorbance compared to the symmetric Si—O—Si peak absorbance. This is an indication of a lower cross-link density coating, which is contemplated to be an unfavorable characteristic for pH protection and long shelf life.

Sample 2 can be particularly compared to Sample 3. A pH protective coating or layer was deposited according to the process conditions of sample 2 in Table 7. The coating was deposited at a low W/FM. This resulted in a coating that exhibited a low Si—O—Si sym/asym ratio of 0.582, which resulted in a high rate of dissolution of 174 ppb/day and short shelf life of 107 days. The FTIR spectrum of this coating exhibits a relatively high asymmetric Si—O—Si peak absorbance compared to the symmetric Si—O—Si peak absorbance. This is an indication of a lower cross-link density coating, which is an unfavorable characteristic for pH protection and long shelf life.

An organosiloxane, pH protective coating or layer was deposited according to the process conditions of sample 3 in Table 7. The coating was deposited at a high W/FM. This resulted in a non-oily coating with a high Si—O—Si sym/asym ratio of 0.947, which resulted in a low rate of Si dissolution of 79.5 ppb/day (following the Protocol for Determining Average Dissolution Rate) and long shelf life of 1381 days (following the Protocol for Determining Calculated Shelf Life). The FTIR spectrum of this coating exhibits a relatively similar asymmetric Si—O—Si peak absorbance compared to the symmetric Si—O—Si peak absorbance. This is an indication of a higher cross-link density coating, which is a preferred characteristic for pH protection and long shelf life.

TABLE 7

| | | | | | | | | | FTIR Absorbance | | | |
|---|---|---|---|---|---|---|---|---|---|---|---|---|
| | | | | | | Si Dissolution @ | | | Si—O—Si | Si—O—Si | | |
| | Process Parameters | | | | | pH8/40° C. | | | sym | asym | Ratio | |
| Flow | | | O₂ | | | Total | Shelf | Rate of | stretch | stretch | Si—O—Si | |
| Rate | | | Flow | Power | W/FM | Si | life | Dissolution | (1000- | (1060- | (sym/ | |
| Samples | OMCTS | Ar | Rate | (W) | (kJ/kg) | (ppb) | (days) | (ppb/day) | 1040 cm⁻¹) | 1100 cm⁻¹) | asym) | Oilyness |
| 1 | 3 | 10 | 0.5 | 14 | 21613 | 43464 | 385 | 293.18 | 0.153 | 0.219 | 0.700 | YES |
| 2 | 3 | 20 | 0.5 | 2 | 3088 | 7180 | 107 | 174.08 | 0.011 | 0.020 | 0.582 | NA |
| 3 | 1 | 20 | 0.5 | 14 | 62533 | 42252.17 | 1381 | 79.53 | 0.093 | 0.098 | 0.947 | NO |
| 4 | 2 | 15 | 0.5 | 8 | 18356 | 27398 | 380 | 187.63 | 0.106 | 0.141 | 0.748 | YES |
| 5 | 3 | 20 | 0.5 | 14 | 21613 | 24699 | 271 | 236.73 | 0.135 | 0.201 | 0.673 | YES |
| 6 | 1 | 10 | 0.5 | 14 | 62533 | 37094 | 1147 | 84.1 | 0.134 | 0.140 | 0.958 | NO |

### Example 17

An experiment similar to Example 14 was carried out, modified as indicated in this example and in Table 8 (where the results are tabulated). 100 5 mL COP vessels were made and coated with an SiOₓ barrier layer and an OMCTS-based pH protective coating or layer as described previously, except that for Sample PC 194 only the pH protective coating or layer was applied. The coating quantity was again measured in parts per billion extracted from the surfaces of the vessels to remove the entire pH protective coating or layer, as reported in Table 8.

In this example, several different coating dissolution conditions were employed. The test solutions used for dissolution contained either 0.02 or 0.2 wt. % polysorbate-80 surfactant, as well as a buffer to maintain a pH of 8. Dissolution tests were carried out at either 23° C. or 40° C.

Multiple vessels were filled with each test solution, stored at the indicated temperature, and analyzed at several intervals to determine the extraction profile and the amount of silicon extracted. An average dissolution rate for protracted storage times was then calculated by extrapolating the data obtained according to the Protocol for Determining Average Dissolution Rate. The results were calculated as described previously and are shown in Table 8. Of particular note, as shown in Table 8, were the very long calculated shelf lives of the filled packages provided with a PC 194 pH protective coating or layer:
21045 days (over 57 years) based on storage at a pH of 8, 0.02 wt. % polysorbate-80 surfactant, at 23° C.;
38768 days (over 100 years) based on storage at a pH of 8, 0.2 wt. % polysorbate-80 surfactant, at 23° C.;
8184 days (over 22 years) based on storage at a pH of 8, 0.02 wt. % polysorbate-80 surfactant, at 40° C.; and
14732 days (over 40 years) based on storage at a pH of 8, 0.2 wt. % polysorbate-80 surfactant, at 40° C.

Referring to Table 8, the longest calculated shelf lives corresponded with the use of an RF power level of 150 Watts and a corresponding high W/FM value. It is believed that the use of a higher power level causes higher cross-link density of the pH protective coating or layer.

TABLE 8

| | OMCTS | Argon | O₂ | | | | Total Si | Calculated | Average |
|---|---|---|---|---|---|---|---|---|---|
| | Flow | Flow | Flow | | Plasma | | (ppb) | Shelf- | Rate of |
| | Rate | Rate | Rate | Power | Duration | W/FM | (OMCTS) | life | Dissolution |
| Sample | (sccm) | (sccm) | (sccm) | (W) | (sec) | (kJ/kg) | layer | (days) | (ppb/day) |
| | Process Parameters | | | | | | Si Dissolution @ pH8/23° C./0.02% | | |
| | | | | | | | Tween ®-80 | | |
| PC194 | 0.5 | 20 | 0.5 | 150 | 20 | 1223335 | 73660 | 21045 | 3.5 |
| 018 | 1.0 | 20 | 0.5 | 18 | 15 | 77157 | 42982 | 1330 | 32.3 |
| PC194 | 0.5 | 20 | 0.5 | 150 | 20 | 1223335 | 73660 | 38768 | 1.9 |
| 018 | 1.0 | 20 | 0.5 | 18 | 15 | 77157 | 42982 | 665 | 64.6 |
| 048 | 4 | 80 | 2 | 35 | 20 | 37507 | 56520 | 1074 | 52.62 |
| PC194 | 0.5 | 20 | 0.5 | 150 | 20 | 1223335 | 73660 | 8184 | 9 |
| 018 | 1.0 | 20 | 0.5 | 18 | 15 | 77157 | 42982 | 511 | 84 |
| PC194 | 0.5 | 20 | 0.5 | 150 | 20 | 1223335 | 73660 | 14732 | 5 |
| 018 | 1.0 | 20 | 0.5 | 18 | 15 | 77157 | 42982 | 255 | 168 |

### Example 18

Another series of experiments similar to those of Example 17 are run, showing the effect of progressively increasing the RF power level on the FTIR absorbance spectrum of the pH protective coating or layer. The results are tabulated in Table 9, which in each instance shows a symmetric/assymetric ratio greater than 0.75 between the maximum amplitude of the Si—O—Si symmetrical stretch peak normally located between about 1000 and 1040 cm−1, and the maximum amplitude of the Si—O—Si asymmetric stretch peak normally located between about 1060 and about 1100 cm−1. Thus, the symmetric/asymmetric ratio is 0.79 at a power level of 20 W, 1.21 or 1.22 at power levels of 40, 60, or 80 W, and 1.26 at 100 Watts under otherwise comparable conditions.

The 150 Watt data in Table 9 is taken under somewhat different conditions than the other data, so it is not directly comparable with the 20-100 Watt data discussed above. The FTIR data of samples 6 and 8 of Table 9 was taken from the upper portion of the vessel and the FTIR data of samples 7 and 9 of Table 9 was taken from the lower portion of the vessel. Also, the amount of OMCTS was cut in half for samples 8 and 9 of Table 9, compared to samples 6 and 7. Reducing the oxygen level while maintaining a power level of 150 W raised the symmetric/asymmetric ratio still further, as shown by comparing samples 6 and 7 to samples 8 and 9 in Table 9.

It is believed that, other conditions being equal, increasing the symmetric/asymmetric ratio increases the shelf life of a vessel filled with a material having a pH exceeding 5.

Table 10 shows the calculated O-Parameters and N-Parameters (as defined in U.S. Pat. No. 8,067,070) for the experiments summarized in Table 9. As Table 10 shows, the O-Parameters ranged from 0.134 to 0.343, and the N-Parameters ranged from 0.408 to 0.623-all outside the ranges claimed in U.S. Pat. No. 8,067,070.

TABLE 9

| | | | | | | | Symmetric | Asymmetric | |
|---|---|---|---|---|---|---|---|---|---|
| | OMCTS | Argon | O₂ | | | | Stretch | Stretch | |
| | Flow | Flow | Flow | | Plasma | | Peak at | Peak at | Symmetric/ |
| Sample | Rate | Rate | Rate | Power | Duration | W/FM | 1000-1040 | 1060-1100 | Assymetric |
| ID | (sccm) | (sccm) | (sccm) | (W) | (sec) | (kJ/kg) | cm⁻¹ | cm⁻¹ | Ratio |
| | Process Parameters | | | | | | FTIR Results | | |
| 1 | 1 | 20 | 0.5 | 20 | 20 | 85,730 | 0.0793 | 0.1007 | 0.79 |
| 2 | 1 | 20 | 0.5 | 40 | 20 | 171,460 | 0.0619 | 0.0507 | 1.22 |
| 3 | 1 | 20 | 0.5 | 60 | 20 | 257,190 | 0.1092 | 0.0904 | 1.21 |
| 4 | 1 | 20 | 0.5 | 80 | 20 | 342,919 | 0.1358 | 0.1116 | 1.22 |
| 5 | 1 | 20 | 0.5 | 100 | 20 | 428,649 | 0.209 | 0.1658 | 1.26 |
| 6 | 1 | 20 | 0.5 | 150 | 20 | 642,973 | 0.2312 | 0.1905 | 1.21 |
| 7 | 1 | 20 | 0.5 | 150 | 20 | 642,973 | 0.2324 | 0.1897 | 1.23 |
| 8 | 0.5 | 20 | 0.5 | 150 | 20 | 1,223,335 | 0.1713 | 0.1353 | 1.27 |
| 9 | 0.5 | 20 | 0.5 | 150 | 20 | 1,223,335 | 0.1475 | 0.1151 | 1.28 |

TABLE 10

| | | | | | | | | |
|---|---|---|---|---|---|---|---|---|
| | OMCTS | Argon | O₂ | | | | | |
| | Flow | Flow | Flow | | Plasma | | | |
| Samples | Rate | Rate | Rate | Power | Duration | W/FM | O— | N— |
| ID | (sccm) | (sccm) | (sccm) | (W) | (sec) | (kJ/kg) | Parameter | Parameter |

Process Parameters

| | | | | | | | | |
|---|---|---|---|---|---|---|---|---|
| 1 | 1 | 20 | 0.5 | 20 | 20 | 85,730 | 0.343 | 0.436 |
| 2 | 1 | 20 | 0.5 | 40 | 20 | 171,460 | 0.267 | 0.408 |
| 3 | 1 | 20 | 0.5 | 60 | 20 | 257,190 | 0.311 | 0.457 |
| 4 | 1 | 20 | 0.5 | 80 | 20 | 342,919 | 0.270 | 0.421 |
| 5 | 1 | 20 | 0.5 | 100 | 20 | 428,649 | 0.177 | 0.406 |
| 6 | 1 | 20 | 0.5 | 150 | 20 | 642,973 | 0.151 | 0.453 |
| 7 | 1 | 20 | 0.5 | 150 | 20 | 642,973 | 0.151 | 0.448 |
| 8 | 0.5 | 20 | 0.5 | 150 | 20 | 1,223,335 | 0.134 | 0.623 |
| 9 | 0.5 | 20 | 0.5 | 150 | 20 | 1,223,335 | 0.167 | 0.609 |

Optionally in any embodiment of the vessel, the barrier coating or layer comprises SiOₓ, where x is from 1.5 to 2.9.

Optionally in any embodiment of the vessel, the barrier coating or layer consists essentially of SiOₓ, where x is from 1.5 to 2.9.

Optionally in any embodiment of the vessel, the barrier coating or layer is deposited by vapor deposition.

Optionally in any embodiment of the vessel, the barrier coating or layer is deposited by chemical vapor deposition.

Optionally in any embodiment of the vessel, the barrier coating or layer is deposited by plasma-enhanced chemical vapor deposition.

Optionally in any embodiment of the vessel, the pH protective coating or layer is deposited by vapor deposition.

Optionally in any embodiment of the vessel, the pH protective coating or layer is deposited by chemical vapor deposition.

Optionally in any embodiment of the vessel, the pH protective coating or layer is deposited by plasma-enhanced chemical vapor deposition.

### Graded Composite Layer

Another expedient contemplated here, for adjacent layers of SiOₓ and a pH protective coating or layer, is a graded composite of any two or more adjacent PECVD layers, for example, the barrier coating or layer 288 and a pH protective coating or layer 286. A graded composite can be separate layers of a pH protective and/or barrier layer or coating with a transition or interface of intermediate composition between them, or separate layers of a protective and/or hydrophobic layer and SiOₓ with an intermediate distinct pH protective coating or layer of intermediate composition between them, or a single coating or layer that changes continuously or in steps from a composition of a protective and/or hydrophobic layer to a composition more like SiOₓ, going through the pH protective coating or layer in a normal direction.

The grade in the graded composite can go in either direction. For example, the composition of SiOₓ can be applied directly to the substrate and graduate to a composition further from the surface of a pH protective coating or layer, and optionally can further graduate to another type of coating or layer, such as a hydrophobic coating or layer or a lubricity coating or layer. Additionally, in any embodiment an adhesion coating or layer, for example, Si_{W}OₓC_{y}, or its equivalent SiOₓC_{y}, optionally can be applied directly to the substrate before applying the barrier layer. A graduated pH protective coating or layer is particularly contemplated if a layer of one composition is better for adhering to the substrate than another, in which case the better-adhering composition can, for example, be applied directly to the substrate. It is contemplated that the more distant portions of the graded pH protective coating or layer can be less compatible with the substrate than the adjacent portions of the graded pH protective coating or layer, since at any point the pH protective coating or layer is changing gradually in properties, so adjacent portions at nearly the same depth of the pH protective coating or layer have nearly identical composition, and more widely physically separated portions at substantially different depths can have more diverse properties. It is also contemplated that a pH protective coating or layer portion that forms a better barrier against transfer of material to or from the substrate can be directly against the substrate, to prevent the more remote pH protective coating or layer portion that forms a poorer barrier from being contaminated with the material intended to be barred or impeded by the barrier.

The applied coatings or layers, instead of being graded, optionally can have sharp transitions between one layer and the next, without a substantial gradient of composition. Such pH protective coating or layer can be made, for example, by providing the gases to produce a layer as a steady-state flow in a non-plasma state, then energizing the system with a brief plasma discharge to form a coating or layer on the substrate. If a subsequent coating or layer is to be applied, the gases for the previous coating or layer are cleared out and the gases for the next coating or layer are applied in a steady-state fashion before energizing the plasma and again forming a distinct layer on the surface of the substrate or its outermost previous coating or layer, with little if any gradual transition at the interface.

### Common Conditions for All Embodiments

In any embodiment contemplated here, many common conditions can be used, for example any of the following, in any combination. For example, the low-pressure PECVD process described in U.S. Pat. No. 7,985,188 can be used. A brief synopsis of that process follows.

The organosilicon precursor for the protective layer can include any of the following precursors useful for PECVD. The precursor for the PECVD coating or layer of the present disclosure is broadly defined as an organometallic precursor. An organometallic precursor is defined in this specification as comprehending compounds of metal elements from Group III and/or Group IV of the Periodic Table having organic residues, for example, hydrocarbon, aminocarbon or oxycarbon residues. Organometallic compounds as presently defined include any precursor having organic moieties bonded to silicon or other Group III/IV metal atoms directly, or optionally bonded through oxygen or nitrogen atoms. The relevant elements of Group III of the Periodic Table are Boron, Aluminum, Gallium, Indium, Thallium, Scandium, Yttrium, and Lanthanum, Aluminum and Boron being preferred. The relevant elements of Group IV of the Periodic Table are Silicon, Germanium, Tin, Lead, Titanium, Zirconium, Hafnium, and Thorium, with Silicon and Tin being preferred. Other volatile organic compounds can also be contemplated. However, organosilicon compounds are preferred for performing present disclosure.

An organosilicon precursor is contemplated, where an “organosilicon precursor” is defined throughout this specification most broadly as a compound having the linkage:

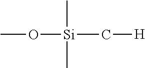

The structure immediately above is a tetravalent silicon atom connected to an oxygen atom and an organic carbon atom (an organic carbon atom being a carbon atom bonded to at least one hydrogen atom). Optionally, the organosilicon precursor is selected from the group consisting of a linear siloxane, a monocyclic siloxane, a polycyclic siloxane, a polysilsesquioxane, a linear silazane, a monocyclic silazane, a polycyclic silazane, a polysilsesquiazane, and a combination of any two or more of these precursors. Also contemplated as a precursor, though not within the two formulas immediately above, is an alkyl trimethoxysilane.

If an oxygen-containing precursor (for example a siloxane) is used, a representative predicted empirical composition resulting from PECVD under conditions forming a coating or layer would be Si_{w}OₓC_{y}H_{z} or its equivalent SiOₓC_{y} as defined in the Definition Section, while a representative predicted empirical composition resulting from PECVD under conditions forming a barrier coating or layer would be SiOₓ, where x in this formula is from about 1.5 to about 2.9.

One type of precursor starting material having the above empirical formula is a linear siloxane, for example, a material having the following formula:

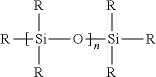

in which each R is independently selected from alkyl, for example, methyl, ethyl, propyl, isopropyl, butyl, isobutyl, t-butyl, vinyl, alkyne, or others, and n is 1, 2, 3, 4, or greater, optionally two or greater. Several examples of contemplated linear siloxanes are hexamethyldisiloxane (HMDSO), octamethyltrisiloxane, decamethyltetrasiloxane, dodecamethylpentasiloxane, or combinations of two or more of these.

Another type of precursor starting material, among the preferred starting materials in the present context, is a monocyclic siloxane, for example, a material having the following structural formula:

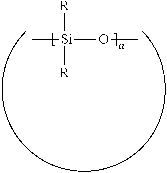

in which R is defined as for the linear structure and “a” is from 3 to about 10, or the analogous monocyclic silazanes. Several examples of contemplated hetero-substituted and unsubstituted monocyclic siloxanes and silazanes include 1,3,5-trimethyl-1,3,5-tris cyclotrisiloxane 2,4,6,8-tetramethyl-2,4,6,8-tetravinylcyclotetrasiloxane, pentamethylcyclopentasiloxane, pentavinylpentamethylcyclopentasiloxane, hexamethylcyclotrisiloxane, hexaphenylcyclotrisiloxane, octamethylcyclotetrasiloxane (OMCTS), octaphenylcyclotetrasiloxane, decamethylcyclopentasiloxane dodecamethylcyclohexasiloxane, methyl(3,3,3-trifluoropropl)cyclosiloxane, or combinations of any two or more of these.

Another type of precursor starting material, among the preferred starting materials in the present context, is a polycyclic siloxane, for example, a material having one of the following structural formulas:

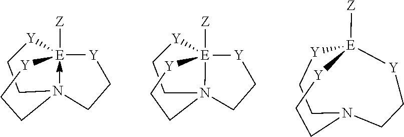

in which Y can be oxygen or nitrogen, E is silicon, and Z is a hydrogen atom or an organic substituent, for example, alkyl such as methyl, ethyl, propyl, isopropyl, butyl, isobutyl, t-butyl, vinyl, alkyne, or others. When each Y is oxygen, the respective structures, from left to right, are a Silatrane, a Silquasilatrane, and a Silproatrane.

Another type of polycyclic siloxane precursor starting material, among the preferred starting materials in the present context, is a polysilsesquioxane, with the empirical formula RSiO_{1.5} and the structural formula:

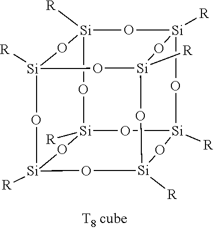

in which each R is a hydrogen atom or an organic substituent, for example alkyl such as methyl, ethyl, propyl, isopropyl, butyl, isobutyl, t-butyl, vinyl, alkyne, or others. Two commercial materials of this sort are SST-eM01 poly(methylsilsesquioxane), in which each R is methyl, and SST-3MH1.1 poly(Methyl-Hydridosilsesquioxane), in which 90% of the R groups are methyl, 10% are hydrogen atoms. This material is available in a 10% solution in tetrahydrofuran, for example. Combinations of two or more of these are also contemplated. Other examples of a contemplated precursor are methylsilatrane, CAS No. 2288-13-3, in which each Y is oxygen and Z is methyl, poly(methylsilsesquioxane) (for example SST-eM01 poly(methylsilsesquioxane)), in which each R optionally can be methyl, SST-3MH1.1 poly(Methyl-Hydridosilsesquioxane) (for example SST-3MH1.1 poly(Methyl-Hydridosilsesquioxane)), in which 90% of the R groups are methyl and 10% are hydrogen atoms, or a combination of any two or more of these.

One particularly contemplated precursor for the barrier coating or layer according to the present disclosure is a linear siloxane, for example, is HMDSO. One particularly contemplated precursor for the pH protective coating or layer according to the present disclosure is a cyclic siloxane, for example, octamethylcyclotetrasiloxane (OMCTS).

It is believed that under certain conditions, the OMCTS or other cyclic siloxane molecule provides several advantages over other siloxane materials. First, its ring structure results in a less dense coating or layer (as compared to a coating or layer prepared from HMDSO). The molecule also allows selective ionization so that the final structure and chemical composition of the coating or layer can be directly controlled through the application of the plasma power. Other organosilicon molecules are readily ionized (fractured) so that it is more difficult to retain the original structure of the molecule.

Under certain other conditions, acyclic siloxane molecules such as TMDSO or HMDSO can instead be used to provide a suitable tie coating or layer 289, barrier coating or layer 288, or pH protective coating or layer 286.

Optionally, the PECVD coating or layer can be formed by chemical vapor deposition of a precursor selected from a monocyclic siloxane, a polycyclic siloxane, a polysilsesquioxane, a silatrane, a silquasilatrane, a silproatrane, or a combination of any two or more of these precursors.

In any of the PECVD methods according to the present disclosure, the applying step optionally can be carried out by vaporizing the precursor and providing it in the vicinity of the substrate. For example, OMCTS is usually vaporized by heating it to about 50° C. before applying it to the PECVD apparatus.

The reaction gas or precursor can also include a hydrocarbon. The hydrocarbon can comprise methane, ethane, ethylene, propane, acetylene, or a combination of two or more of these.

The organosilicon precursor can be delivered at a rate of equal to or less than 10 sccm, optionally equal to or less than 6 sccm, optionally equal to or less than 2.5 sccm, optionally equal to or less than 1.5 sccm, optionally equal to or less than 1.25 sccm. Larger pharmaceutical packages or other vessels or other changes in conditions or scale may require more or less of the precursor. The precursor can be provided at less than 1 Torr absolute pressure.

### Other Components of PECVD Reaction Mixture and Ratios of Components

Generally, for a tie coating or layer 289 or pH protective coating or layer 286, O₂ can be present in an amount (which can, for example be expressed by the flow rate in sccm) which is less than, or less than one order of magnitude greater than, the organosilicon amount. In contrast, in order to achieve a barrier coating or layer, the amount of O₂ typically is at least one order of magnitude higher than the amount of organosilicon precursor. In particular, the volume ratio (in sccm) of organosilicon precursor to O₂ for a tie coating or layer 289 or pH protective coating or layer 286, can be in the range from 0.1:1 to 10:1, optionally in the range from 0.3:1 to 8:1, optionally in the range from 0.5:1 to 5:1, optionally from 1:1 to 3:1. The presence of the precursor and O₂ in the volume ratios as given in the working examples is specifically suitable to achieve a tie coating or layer 289 or pH protective coating or layer 286.

### Carrier Gas of any Embodiment

The tie coating or layer 289 or pH protective coating or layer 286 optionally can be made using a carrier gas. The carrier gas, alternatively referred to as a diluent gas since it is not used to take up or entrain the precursor in the illustrated embodiments, can comprise or consist of an inert gas, for example argon, helium, xenon, neon, another gas that is inert to the other constituents of the process gas under the deposition conditions, or any combination of two or more of these.

In one aspect, a carrier gas is absent in the reaction mixture, in another aspect, it is present. Suitable carrier gases include Argon, Helium and other noble gases such as Neon and Xenon. When the carrier gas is present in the reaction mixture, it is typically present in a volume (in sccm) exceeding the volume of the organosilicon precursor. For example, the ratio of the organosilicon precursor to carrier gas can be from 1:1 to 1:50, optionally from 1:5 to 1:40, optionally from 1:10 to 1:30. One function of the carrier gas is to dilute the reactants in the plasma, encouraging the formation of a coating on the substrate instead of powdered reaction products that do not adhere to the substrate and are largely removed with the exhaust gases.

Since the addition of Argon gas improves the protective performance (see the working examples below), it is believed that additional ionization of the molecule in the presence of Argon contributes to providing a tie coating or layer 289 or pH protective coating or layer 286. The Si—O—Si bonds of the molecule have a high bond energy followed by the Si—C, with the C—H bonds being the weakest. A tie coating or layer 289 or pH protective coating or layer 286, appears to be achieved when a portion of the C—H bonds are broken. This allows the connecting (cross-linking) of the structure as it grows. Addition of oxygen (with the Argon) is understood to enhance this process. A small amount of oxygen can also provide C—O bonding to which other molecules can bond. The combination of breaking C—H bonds and adding oxygen all at low pressure and power leads to a chemical structure that is solid while providing a tie coating or layer 289 or pH protective coating or layer 286.

### Oxidizing Gas of any Embodiment

The oxidizing gas can comprise or consist of oxygen (O₂ and/or O₃ (commonly known as ozone)), nitrous oxide, or any other gas that oxidizes the precursor during PECVD at the conditions employed. The oxidizing gas comprises about 1 standard volume of oxygen. The gaseous reactant or process gas can be at least substantially free of nitrogen.

In any of embodiments, one preferred combination of process gases includes octamethylcyclotetrasiloxane (OMCTS) or another cyclic siloxane as the precursor, in the presence of oxygen as an oxidizing gas and argon as a carrier gas. Without being bound to the accuracy of this theory, the inventors believe this particular combination is effective for the following reasons. The presence of O₂, N₂O, or another oxidizing gas and/or of a carrier gas, in particular of a carrier gas, for example a noble gas, for example Argon (Ar), is contemplated to improve the resulting pH protective coating or layer.

Some non-exhaustive alternative selections and suitable proportions of the precursor gas, oxygen, and a carrier gas are provided below.
OMCTS: 0.5-5.0 sccm
Oxygen: 0.1-5.0 sccm
Argon: 1.0-20 sccm

### RF POWER OF ANY EMBODIMENT

The precursor can be contacted with a plasma made by energizing the vicinity of the precursor with electrodes powered at a frequency of 10 kHz to 2.45 GHz, alternatively from about 13 to about 14 MHz.

The precursor can be contacted with a plasma made by energizing the vicinity of the precursor with electrodes powered at radio frequency, optionally at a frequency of from 10 kHz to less than 300 MHz, optionally from 1 to 50 MHz, even optionally from 10 to 15 MHz, optionally at 13.56 MHz.

The precursor can be contacted with a plasma made by energizing the vicinity of the precursor with electrodes supplied with electric power at from 0.1 to 25 W, optionally from 1 to 22 W, optionally from 1 to 10 W, even optionally from 1 to 5 W, optionally from 2 to 4 W, for example of 3 W, optionally from 3 to 17 W, even optionally from 5 to 14 W, for example 6 or 7.5 W, optionally from 7 to 11 W, for example of 8 W, from 0.1 to 500 W, optionally from 0.1 to 400 W, optionally from 0.1 to 300 W, optionally from 1 to 250 W, optionally from 1 to 200 W, even optionally from 10 to 150 W, optionally from 20 to 150 W, for example of 40 W, optionally from 40 to 150 W, even optionally from 60 to 150 W.

The precursor can be contacted with a plasma made by energizing the vicinity of the precursor with electrodes supplied with electric power density at less than 10 W/ml of plasma volume, alternatively from 6 W/ml to 0.1 W/ml of plasma volume, alternatively from 5 W/ml to 0.1 W/ml of plasma volume, alternatively from 4 W/ml to 0.1 W/ml of plasma volume, alternatively from 2 W/ml to 0.2 W/ml of plasma volume, alternatively from 10 W/ml to 50 W/ml, optionally from 20 W/ml to 40 W/ml.

The plasma can be formed by exciting the reaction mixture with electromagnetic energy, alternatively microwave energy.

### OTHER PROCESS OPTIONS OF ANY EMBODIMENT

The applying step for applying a coating or layer to the substrate can be carried out by vaporizing the precursor and providing it in the vicinity of the substrate.

The chemical vapor deposition employed can be PECVD and the deposition time can be from 1 to 30 sec, alternatively from 2 to 10 sec, alternatively from 3 to 9 sec. The purposes for optionally limiting deposition time can be to avoid overheating the substrate, to increase the rate of production, and to reduce the use of process gas and its constituents. The purpose for optionally extending deposition time can be to provide a thicker coating or layer for particular deposition conditions.

### GASEOUS REACTANT OR PROCESS GAS LIMITATIONS OF ANY EMBODIMENT

The plasma for PECVD, if used, can be generated at reduced pressure and the reduced pressure can be less than 300 mTorr, optionally less than 200 mTorr, even optionally less than 100 mTorr. The physical and chemical properties of the tie coating or layer 289, the pH protective coating or layer 286, or a layer serving the function of more than one of these can be set by setting the ratio of O₂ to the organosilicon precursor in the gaseous reactant, and/or by setting the electric power used for generating the plasma. The plasma of any PECVD embodiment can be formed in the vicinity of the substrate. The plasma can in certain cases, especially when preparing a barrier coating or layer, be a non-hollow-cathode plasma. In other certain cases, especially when preparing the tie coating or layer 289, the pH protective coating or layer 286, or a layer serving the function of more than one of these a non-hollow-cathode plasma is not desired. The plasma can be formed from the gaseous reactant at reduced pressure. Sufficient plasma generation power input can be provided to induce coating or layer formation on the substrate.

Relative proportions of gases for producing the tie coating or layer 289 or the pH protective coating or layer 286.

The process gas can contain this ratio of gases for preparing the tie coating or layer 289, the pH protective coating or layer 286, or a layer serving the function of more than one of these:
from 0.5 to 10 standard volumes of the precursor;
from 1 to 100 standard volumes of a carrier gas,
from 0.1 to 10 standard volumes of an oxidizing agent.
   alternatively this ratio:
from 1 to 6 standard volumes of the precursor;
from 1 to 80 standard volumes of a carrier gas,
from 0.1 to 2 standard volumes of an oxidizing agent.
   alternatively this ratio:
from 2 to 4 standard volumes, of the precursor;
from 1 to 100 standard volumes of a carrier gas,
from 0.1 to 2 standard volumes of an oxidizing agent.
   alternatively this ratio:
from 1 to 6 standard volumes of the precursor;
from 3 to 70 standard volumes, of a carrier gas,
from 0.1 to 2 standard volumes of an oxidizing agent.
   alternatively this ratio:
from 2 to 4 standard volumes, of the precursor;
from 3 to 70 standard volumes of a carrier gas,
from 0.1 to 2 standard volumes of an oxidizing agent.
   alternatively this ratio:
from 1 to 6 standard volumes of the precursor;
from 1 to 100 standard volumes of a carrier gas,
from 0.2 to 1.5 standard volumes of an oxidizing agent.
   alternatively this ratio:
from 2 to 4 standard volumes, of the precursor;
from 1 to 100 standard volumes of a carrier gas,
from 0.2 to 1.5 standard volumes of an oxidizing agent.
   alternatively this ratio:
from 1 to 6 standard volumes of the precursor;
from 3 to 70 standard volumes of a carrier gas,
from 0.2 to 1.5 standard volumes of an oxidizing agent.
   alternatively this ratio:
from 2 to 4 standard volumes of the precursor;
from 3 to 70 standard volumes of a carrier gas,
from 0.2 to 1.5 standard volumes of an oxidizing agent.
   alternatively this ratio:
from 1 to 6 standard volumes of the precursor;
from 1 to 100 standard volumes of a carrier gas,
from 0.2 to 1 standard volume of an oxidizing agent.
   alternatively this ratio:
from 2 to 4 standard volumes of the precursor;
from 1 to 100 standard volumes of a carrier gas,
from 0.2 to 1 standard volume of an oxidizing agent.
   alternatively this ratio:
from 1 to 6 standard volumes of the precursor;
from 3 to 70 standard volumes of a carrier gas,
from 0.2 to 1 standard volume of an oxidizing agent.
   alternatively this ratio:
2 to 4 standard volumes, of the precursor;
from 3 to 70 standard volumes of a carrier gas,
from 0.2 to 1 standard volume of an oxidizing agent.
   alternatively this ratio:
from 1 to 6 standard volumes of the precursor;
from 5 to 100 standard volumes of a carrier gas,
from 0.1 to 2 standard volumes of an oxidizing agent.
   alternatively this ratio:
from 2 to 4 standard volumes, of the precursor;
from 5 to 100 standard volumes of a carrier gas,
from 0.1 to 2 standard volumes of an oxidizing agent.
   alternatively this ratio:
from 1 to 6 standard volumes of the precursor;
from 10 to 70 standard volumes, of a carrier gas,
from 0.1 to 2 standard volumes of an oxidizing agent.
   alternatively this ratio:
from 2 to 4 standard volumes, of the precursor;
from 10 to 70 standard volumes of a carrier gas,
from 0.1 to 2 standard volumes of an oxidizing agent.
   alternatively this ratio:
from 1 to 6 standard volumes of the precursor;
from 5 to 100 standard volumes of a carrier gas,
from 0.5 to 1.5 standard volumes of an oxidizing agent.
   alternatively this ratio:
from 2 to 4 standard volumes, of the precursor;
from 5 to 100 standard volumes of a carrier gas,
from 0.5 to 1.5 standard volumes of an oxidizing agent.
   alternatively this ratio:
from 1 to 6 standard volumes of the precursor;
from 10 to 70 standard volumes, of a carrier gas,
from 0.5 to 1.5 standard volumes of an oxidizing agent.
   alternatively this ratio:
from 2 to 4 standard volumes of the precursor;
from 10 to 70 standard volumes of a carrier gas,
from 0.5 to 1.5 standard volumes of an oxidizing agent.
   alternatively this ratio:
from 1 to 6 standard volumes of the precursor;
from 5 to 100 standard volumes of a carrier gas,
from 0.8 to 1.2 standard volumes of an oxidizing agent.
   alternatively this ratio:
from 2 to 4 standard volumes of the precursor;
from 5 to 100 standard volumes of a carrier gas,
from 0.8 to 1.2 standard volumes of an oxidizing agent.
   alternatively this ratio:
from 1 to 6 standard volumes of the precursor;
from 10 to 70 standard volumes of a carrier gas,
from 0.8 to 1.2 standard volumes of an oxidizing agent.
   alternatively this ratio:
2 to 4 standard volumes, of the precursor;
from 10 to 70 standard volumes of a carrier gas,
from 0.8 to 1.2 standard volumes of an oxidizing agent.

### Additional Embodiments

The tie coating or layer 289, the pH protective coating or layer 286, or a layer serving the function of more than one of these described in this specification can be applied in many different ways. For one example, the low-pressure PECVD process described in U.S. Pat. No. 7,985,188 can be used. For another example, instead of using low-pressure PECVD, atmospheric PECVD can be employed to deposit the tie coating or layer 289, the pH protective coating or layer 286, or a layer serving the function of more than one of these. For another example, the injection moulding may prepare the coatings at approximately 135 Mpa. For another example, the coating can be simply evaporated and allowed to deposit on the SiOₓ layer to be protected. For another example, the coating can be sputtered on the SiOₓ layer to be protected. In yet another example, the blood collections tubes may include a variety of sizes of tubes. For example, a first blood collection tube may be molded with a nominal fill volume of 4 ml (including a 13 mm outside diameter O.D. and 74 mm height) and a second blood collection tube may be moulded with a nominal fill volume of 9 ml (including a 16 mm outside diameter O.D. and 100 mm height).

### Process for Applying Tie Coating or Layer 289

The general PECVD process described in this specification is used, for example, as follows, to produce a tie or adhesion coating or layer 289 (several names for the same type of coating) on a 1 to 5 mL vessel such as a pharmaceutical vessel or prefilled syringe. A person skilled in the art will know from this description how to scale the process conditions to suit larger or smaller vessels.

The tie or adhesion coating or layer can be produced, for example, using as the precursor tetramethyldisiloxane (TMDSO) or hexamethyldisiloxane (HMDSO) at a flow rate of 0.5 to 10 sccm, preferably 1 to 5 sccm; oxygen flow of 0.25 to 5 sccm, preferably 0.5 to 2.5 sccm; and argon flow of 1 to 120 sccm, preferably in the upper part of this range for a 1 mL vessel and the lower part of this range for a 5 ml. vessel. Further, the gas mixture of the precursor, oxygen and argon flows continuously in and out of the blood tube under steady-state flow conditions. The overall pressure in the vessel during PECVD can be from 0.01 to Torr, preferably from 0.1 to 1.5 Torr. The power level applied can be from 5 to 100 Watts, preferably in the upper part of this range for a 1 mL vessel and the lower part of this range for a 5 ml. vessel. The deposition time (i.e. “on” time for RF power) is from 0.1 to 10 seconds, preferably 1 to 3 seconds. The power cycle optionally can be ramped or steadily increased from 0 Watts to full power over a short time period, such as 2 seconds, when the power is turned on, which may improve the plasma uniformity. The ramp-up of power over a period of time is optional, however.

### PECVD Process for Applying Barrier Layer

The PECVD processes described as suitable in U.S. Pat. No. 7,985,188, incorporated by reference here, can be used to apply an SiOₓ barrier coating or layer 288 as defined in this specification.

### PECVD Process for pH Protective Layer

A tie coating or layer 289 or a pH protective coating or layer 286 can be a SiOₓC_{y} coating or layer applied as described in any embodiment of this specification. For example, the pH protective coating or layer 286 of any embodiment comprises or consists essentially of a coating or layer of SiOₓC_{y} optionally applied over the barrier coating or layer 288 to protect at least a portion of the barrier coating or layer from the pharmaceutical preparation such as 218. The pH protective coating or layer such as 286 is provided, for example, by applying one of the described precursors on or in the vicinity of a substrate in a PECVD process, providing a pH protective coating or layer. The coating can be applied, for example, at a thickness of 1 to 5000 nm, or 10 to 1000 nm, or 10 to 500 nm, or 10 to 200 nm, or 20 to 100 nm, or 30 to 1000 nm, or 30 to 500 nm thick, or 30 to 1000 nm, or 20 to 100 nm, or 80 to 150 nm, and crosslinking or polymerizing (or both) the protective layer, optionally in a PECVD process, to provide a protected surface.

Although not intending to be bound according to the accuracy of the following theory, the inventors contemplate that the pH protective coating or layer 286, applied over an SiOₓ barrier layer on a vessel wall, functions at least in part by passivating the SiOₓ barrier layer surface against attack by the contents of the vessel, as well as providing a more resistant or sacrificial independent layer to isolate the SiOₓ barrier layer from the contents of the vessel. It is thus contemplated that the pH protective coating or layer can be very thin, and even so improve the shelf life of the pharmaceutical package.

Exemplary reaction conditions for preparing a pH protective coating or layer 286 coating or layer according to the present disclosure in a 3 ml sample size vessel with a ⅛″ diameter tube (open at the end) are as follows:

Flow rate ranges:
OMCTS: 0.5-10 sccm
Oxygen: 0.1-10 sccm
Argon: 1.0-200 sccm
Power: 0.1-500 watts
   Specific Flow rates:
OMCTS: 2.0 sccm
Oxygen: 0.7 sccm
Argon: 7.0 sccm
Power: 3.5 watts

The pH protective coating or layer 286 and its application are described in more detail below. A method for applying the coating includes several steps. A vessel wall is provided, as is a reaction mixture comprising plasma forming gas, i.e., an organosilicon compound gas, optionally an oxidizing gas, and optionally a hydrocarbon gas.

Plasma is formed in the reaction mixture that is substantially free of hollow cathode plasma. The vessel wall is contacted with the reaction mixture, and the coating or layer of SiOₓ is deposited on at least a portion of the vessel wall.

It has been found that acyclic organosiloxanes, for example, HMDSO and TMDSO, can be used to form the pH protective coating or layer, as described elsewhere in this specification.

In certain embodiments, the generation of a uniform plasma throughout the portion of the vessel to be coated is contemplated, as it has been found in certain instances to generate a better coating or layer. Uniform plasma means regular plasma that does not include a substantial amount of hollow cathode plasma (which has a higher emission intensity than regular plasma and is manifested as a localized area of higher intensity interrupting the more uniform intensity of the regular plasma).

### Additional Embodiments

The pH protective coating or layer 286 described in this specification can be applied in many different ways. For one example, the low-pressure PECVD process described in U.S. Pat. No. 7,985,188 can be used. For another example, instead of using low-pressure PECVD, atmospheric PECVD can be employed to deposit the pH protective coating or layer. For another example, the coating can be simply evaporated and allowed to deposit on the SiOₓ layer to be protected. For another example, the coating can be sputtered on the SiOₓ layer to be protected. For still another example, the pH protective coating or layer 286 can be applied from a liquid medium used to rinse or wash the SiOₓ layer.

Other precursors and methods can be used to apply the pH protective coating or layer or passivating treatment. For example, hexamethylene disilazane (HMDZ) can be used as the precursor. HMDZ has the advantage of containing no oxygen in its molecular structure. This passivation treatment is contemplated to be a surface treatment of the SiOₓ barrier layer with HMDZ. To slow down and/or eliminate the decomposition of the silicon dioxide coatings at silanol bonding sites, the coating must be passivated. It is contemplated that passivation of the surface with HMDZ (and optionally application of a few mono layers of the HMDZ-derived coating) will result in a toughening of the surface against dissolution, resulting in reduced decomposition. It is contemplated that HMDZ will react with the —OH sites that are present in the silicon dioxide coating, resulting in the evolution of NH3 and bonding of S—(CH3)3 to the silicon (it is contemplated that hydrogen atoms will be evolved and bond with nitrogen from the HMDZ to produce NH3).

It is contemplated that this HMDZ passivation can be accomplished through several possible paths.

One contemplated path is dehydration/vaporization of the HMDZ at ambient temperature. First, an SiOₓ surface is deposited, for example, using hexamethylene disiloxane (HMDSO). The as-coated silicon dioxide surface is then reacted with HMDZ vapor. In an embodiment, as soon as the SiOₓ surface is deposited onto the article of interest, the vacuum is maintained. The HMDSO and oxygen are pumped away and a base vacuum is achieved. Once base vacuum is achieved, HMDZ vapor is flowed over the surface of the silicon dioxide (as coated on the part of interest) at pressures from the mTorr range to many Torr. The HMDZ is then pumped away (with the resulting NH3 that is a by-product of the reaction). The amount of NH₃ in the gas stream can be monitored (with a residual gas analyser—RGA—as an example) and when there is no more NH₃ detected, the reaction is complete. The part is then vented to atmosphere (with a clean dry gas or nitrogen). The resulting surface is then found to have been passivated. It is contemplated that this method optionally can be accomplished without forming a plasma.

Alternatively, after formation of the SiOₓ barrier coating or layer, the vacuum can be broken before dehydration/vaporization of the HMDZ. Dehydration/vaporization of the HMDZ can then be carried out in either the same apparatus used for formation of the SiOₓ barrier coating or layer or different apparatus.

Dehydration/vaporization of HMDZ at an elevated temperature is also contemplated. The above process can alternatively be carried out at an elevated temperature exceeding room temperature up to about 150° C. The maximum temperature is determined by the material from which the coated part is constructed. An upper temperature should be selected that will not distort or otherwise damage the part being coated.

Dehydration/vaporization of HMDZ with a plasma assist is also contemplated. After carrying out any of the above embodiments of dehydration/vaporization, once the HMDZ vapor is admitted into the part, a plasma is generated. The plasma power can range from a few watts to 100+watts (similar power as used to deposit the SiOₓ). The above is not limited to HMDZ and could be applicable to any molecule that will react with hydrogen, for example, any of the nitrogen-containing precursors described in this specification.

For depositing a pH protective coating or layer, a precursor feed or process gas can be employed having a standard volume ratio of, for example:
from 0.5 to 10 standard volumes, optionally from 1 to 6 standard volumes, optionally from 2 to 4 standard volumes, optionally equal to or less than 6 standard volumes, optionally equal to or less than 2.5 standard volumes, optionally equal to or less than 1.5 standard volumes, optionally equal to or less than 1.25 standard volumes of the precursor, for example, OMCTS or one of the other precursors of any embodiment;
from 0 to 100 standard volumes, optionally from 1 to 80 standard volumes, optionally from 5 to 100 standard volumes, optionally from 10 to 70 standard volumes, of a carrier gas of any embodiment;
from 0.1 to 10 standard volumes, optionally from 0.1 to 2 standard volumes, optionally from 0.2 to 1.5 standard volumes, optionally from 0.2 to 1 standard volume, optionally from 0.5 to 1.5 standard volumes, optionally from 0.8 to 1.2 standard volumes of an oxidizing agent.

Another embodiment is a pH protective coating or layer of the type made by the above process.

Another embodiment is a vessel such as the vessel 214 (FIG. 1) including a lumen defined by a surface defining a substrate. A pH protective coating or layer is present on at least a portion of the substrate, typically deposited over an SiOₓ barrier layer to protect the barrier layer from dissolution. The pH protective coating or layer is made by the previously defined process.

### PECVD Process for Trilayer Coating

The PECVD trilayer coating described in this specification can be applied, for example, as follows for a 1 to 5 mL vessel. Two specific examples are 1 mL thermoplastic resin vessel and a 5 mL thermoplastic resin drug vessel. Larger or smaller vessels will call for adjustments in parameters that a person of ordinary skill can carry out in view of the teaching of this specification.

The apparatus used is the PECVD apparatus with rotating quadrupole magnets as described generally in this specification.

The general coating parameter ranges, with preferred ranges in parentheses, for a trilayer coating for a 1 mL vessel are shown in the PECVD Trilayer Process General Parameters Tables (1 mL vessel and 5 mL vessel).

PECVD Trilayer Process General Parameters Table (1 mL vessel)

| Parameter | Units | Tie | Barrier | pH Protective |
|---|---|---|---|---|
| Power | W | 40-90 | 140 | 40-90 |
| | | (60-80) | | (60-80) |
| TMDSO Flow | sccm | 1-10 | None | 1-10 |
| | | (3-5) | | (3-5) |
| HMDSO Flow | sccm | None | 1.56 | None |
| O₂ Flow | sccm | 0.5-5 | 20 | 0.5-5 |
| | | (1.5-2.5) | | (1.5-2.5) |
| Argon Flow | sccm | 40-120 | 0 | 40-120 |
| | | (70-90) | | (70-90) |
| Ramp Time | seconds | None | None | None |
| Deposition Time | seconds | 0.1-10 | 20 | 0.1-40 |
| | | (1-3) | | (15-25) |
| Tube Pressure | Torr | 0.01-10 | 0.59 | 0.01-10 |
| | | (0.1-1.5) | | (0.1-1.5) |

PECVD Trilayer Process General Parameters Table (5 mL vessel)

| Parameter | Units | Adhesion | Barrier | Protection |
|---|---|---|---|---|
| Power | W | 40-90 | 140 | 40-90 |
| | | (60-80) | | (60-80) |
| TMDSO Flow | sccm | 1-10 | None | 1-10 |
| | | (3-5) | | (3-5) |
| HMDSO Flow | sccm | None | 1.56 | None |
| O₂ Flow | sccm | 0.5-5 | 20 | 0.5-5 |
| | | (1.5-2.5) | | (1.5-2.5) |
| Argon Flow | sccm | 40-120 | 0 | 40-120 |
| | | (70-90) | | (70-90) |
| Ramp Time | seconds | None | None | None |
| Deposition Time | seconds | 0.1-10 | 20 | 0.1-40 |
| | | (1-3) | | (15-25) |
| Tube Pressure | Torr | 0.01-10 | 0.59 | 0.01-10 |
| | | (0.1-1.5) | | (0.1-1.5) |

### Trilayer Working Examples

Examples of specific coating parameters that have been used for a 1 mL vessel and 5 mL vessel are shown in the PECVD Trilayer Process Specific Parameters Tables (1 mL vessel and 5 mL vessel):

PECVD Trilayer Process Specific Parameters Table (1 mL syringe)

| Parameter | Units | Tie | Barrier | Protection |
|---|---|---|---|---|
| Power | W | 70 | 140 | 70 |
| TMDSO Flow | sccm | 4 | None | 4 |
| HMDSO Flow | sccm | None | 1.56 | None |
| O₂ Flow | sccm | 2 | 20 | 2 |
| Argon Flow | sccm | 80 | 0 | 80 |
| Ramp Time | seconds | None | None | None |
| Deposition Time | seconds | 2.5 | 20 | 10 |
| Tube Pressure | Torr | 1 | 0.59 | 1 |

PECVD Trilayer Process Specific Parameters Table (5 mL vessel)

| Parameter | Units | Adhesion | Barrier | Protection |
|---|---|---|---|---|
| Power | W | 20 | 40 | 20 |
| TMDSO Flow | sccm | 2 | 0 | 2 |
| HMDSO Flow | sccm | 0 | 3 | 0 |
| O₂ Flow | sccm | 1 | 50 | 1 |
| Argon Flow | sccm | 20 | 0 | 20 |
| Ramp Time | seconds | 0 | 2 | 2 |
| Deposition Time | seconds | 2.5 | 10 | 10 |
| Tube Pressure | Torr | 0.85 | 1.29 | 0.85 |

The O-parameter and N-parameter values for the pH protective coating or layer applied to the 1 mL vessel as described above are 0.34 and 0.55, respectively.

The O-parameter and N-parameter values for the pH protective coating or layer applied to the 5 mL vessel are 0.24 and 0.63, respectively.

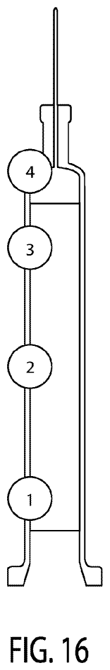

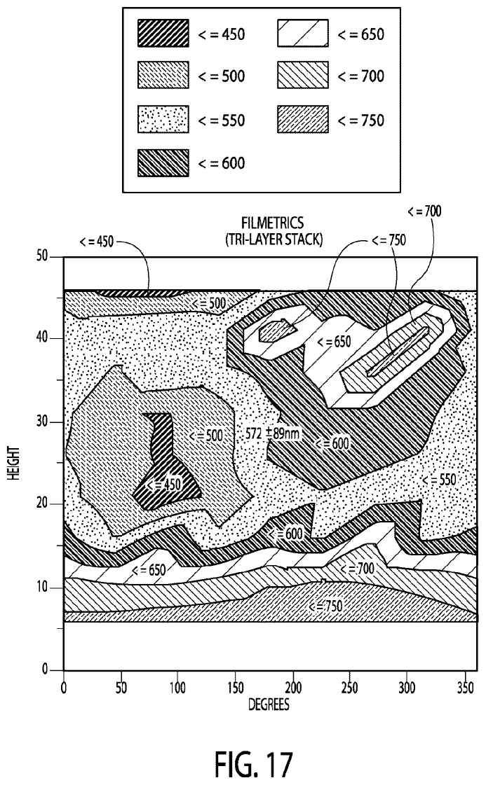

Referring to FIGS. 16-18, the thickness uniformity at four different points along the length of a 1 mL vessel with a staked needle as the vessel (present during PECVD deposition) and the indicated trilayer coating (avg. thicknesses: 38 nm adhesion or tie coating or layer; 55 nm barrier coating or layer, 273 nm pH protective coating or layer) is shown. The plot maps the coating thickness over the cylindrical inner surface of the barrel, as though unrolled to form a rectangle. The overall range is 572 plus or minus 89 nm. The table shows individual layer thicknesses at the four marked points, showing adequate thickness of each layer at each point along the high profile vessel.

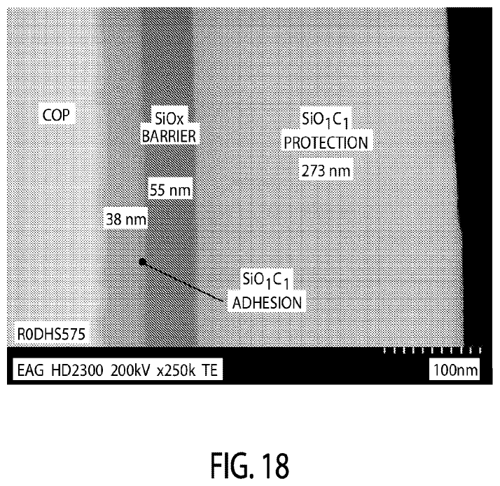

A vessel having a coating similar to the trilayer coating of FIG. 18 is tested for shelf life, using the silicon dissolution and extrapolation method described in this specification, compared to vessels having a bilayer coating (similar to the trilayer coating except lacking the tie coating or layer) and a monolayer coating which is just the pH protective coating or layer directly applied to the thermoplastic barrel of the vessel, with no barrier layer. The test solution was a 0.2% Tween, pH 8 phosphate buffer. The extrapolated shelf lives of the monolayer and trilayer coatings were similar and very long—on the order of 14 years. The shelf life of the vessels having a bilayer coating were much lower—less than two years. In other words, the presence of a barrier layer under the pH protective layer shortened the shelf life of the coating substantially, but the shelf life was restored by providing a tie coating or layer under the barrier layer, sandwiching the barrier coating or layer with respective SiOₓC_{y} layers. The barrier layer is necessary to establish a gas barrier, so the monolayer coating would not be expected to provide adequate gas barrier properties by itself. Thus, only the trilayer coating had the combination of gas barrier properties and a long shelf life, even while in contact with a solution that would attack an exposed barrier coating or layer.

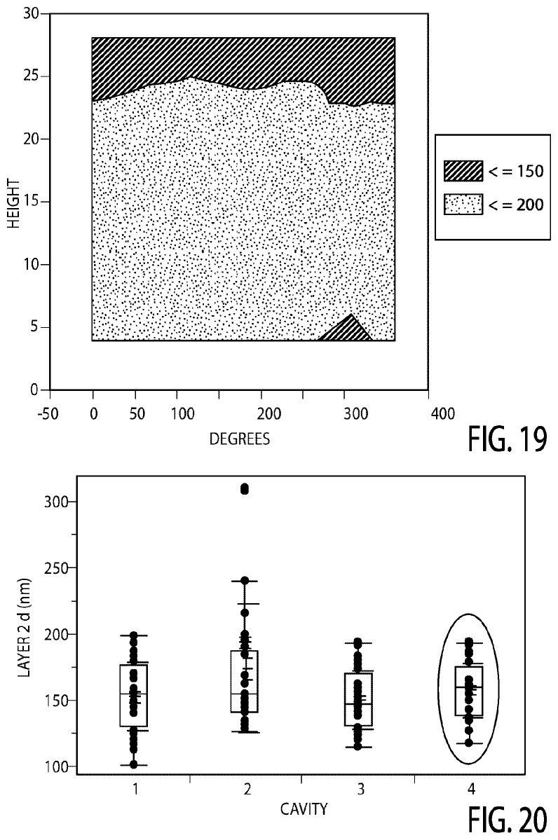

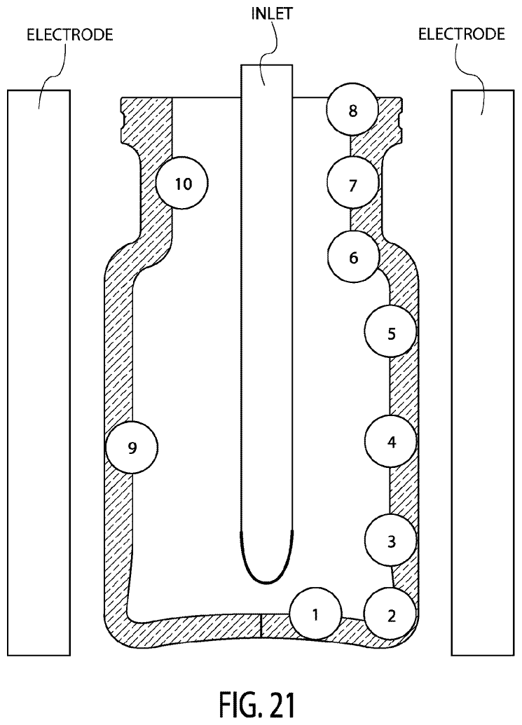

FIGS. 19-20 show the coating distribution for a 5 mL vessel—a vial similar to that of FIG. 21—showing very little variation in coating thickness, with the great majority of the surface coated between 150 and 250 nm thickness of the trilayer, with only a small proportion of the container coated with between 50 and 250 nm of the trilayer.

The Vessel Coating Distribution Table shows the breakdown of coating thickness (nm) by vessel location as shown in FIG. 21. The Vessel Coating Distribution Table shows the uniformity of coating.

Vessel Coating Distribution Table

| | | | | Total |
|---|---|---|---|---|
| Vessel Location | Adhesion | Barrier | Protection | Trilayer, nm |
| 1 | 13 | 29 | 77 | 119 |
| 2 | 14 | 21 | 58 | 93 |
| 3 | 25 | 37 | 115 | 177 |
| 4 | 35 | 49 | 158 | 242 |
| 5 | 39 | 49 | 161 | 249 |
| 6 | 33 | 45 | 148 | 226 |
| 7 | 31 | 29 | 153 | 213 |
| 8 | 48 | 16 | 218 | 282 |
| 9 | 33 | 53 | 155 | 241 |
| 10 | 31 | 29 | 150 | 210 |
| Average | 30 | 36 | 139 | 205 |

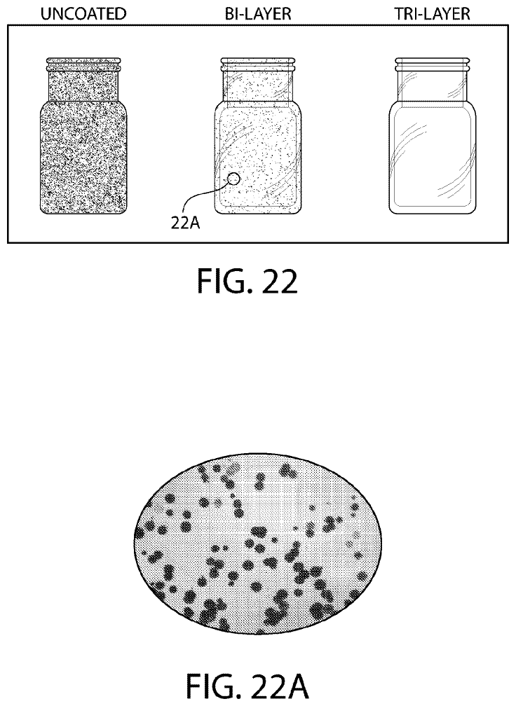

FIG. 22 is a visual test result showing the integrity of the trilayer vessel coating described above. The three 5 mL cyclic olefin polymer (COC) vessels of FIG. 22 were respectively:
uncoated (left vessel),
coated with the bilayer coating described in this specification (a barrier coating or layer plus a pH protective coating or layer—the second and third components of the trilayer coating) (center vessel); and
coated with the trilayer coating as described above (right vessel).

The three vessels were each exposed to 1 N potassium hydroxide for four hours, then exposed for 24 hours to a ruthenium oxide (RuO₄) stain that darkens any exposed part of the thermoplastic vessel unprotected by the coatings. The high pH potassium hydroxide exposure erodes any exposed part of the barrier coating or layer at a substantial rate, greatly reduced, however by an intact pH protective coating or layer. In particular, the high pH exposure opens up any pinholes in the coating system. As FIG. 22 shows, the uncoated vessel is completely black, showing the absence of any effective coating. The bilayer coating (Center, FIG. 22, portion enlarged in FIG. 22A) was mostly intact under the treatment conditions, but on microscopic inspection has many pinholes where the ruthenium stain reached the thermoplastic substrate through the coating. The overall appearance of the bilayer coating clearly shows visible “soiled” areas where the stain penetrated. The trilayer coating, however (FIG. 22, Right), protected the entire vessel against penetration of the stain, and the illustrated vessel remains clear after treatment. This is believed to be the result of sandwiching the barrier coating or layer between two layers of SiOₓC_{y}, which both protects the barrier layer against direct etching and against undercutting and removal of flakes of the barrier layer.

The rate of erosion of the pH protective coating or layer 286, if directly contacted by the fluid 218, is less than the rate of erosion of the barrier coating 288, if directly contacted by the fluid 218.

The pH protective coating or layer 286 is effective to isolate the fluid 218 from the barrier coating 288.

Optionally for any of the embodiments, at least a portion of the wall 214 of the vessel 250 comprises or consists essentially of a polymer, for example, a polyolefin (for example a cyclic olefin polymer, a cyclic olefin copolymer

Optionally for any of the embodiments, the fluid 218 in the lumen such as 212 or 274 has a pH between 5 and 6, optionally between 6 and 7, optionally between 7 and 8, optionally between 8 and 9, optionally between 6.5 and 7.5, optionally between 7.5 and 8.5, optionally between 8.5 and 9.

Optionally for any of the embodiments, the fluid 218 is a liquid at 20° C. and ambient pressure at sea level, which is defined as a pressure of 760 mm Hg.

Optionally for any of the embodiments, the fluid 218 is an aqueous liquid.

Optionally for any of the embodiments, the barrier coating 288 is from 4 nm to 500 nm thick, optionally from 7 nm to 400 nm thick, optionally from 10 nm to 300 nm thick, optionally from 20 nm to 200 nm thick, optionally from 30 nm to 100 nm thick.

Optionally for any of the embodiments, the pH protective coating or layer 286 comprises or consists essentially of SiOₓC_{y}. Optionally for any of the embodiments, the pH protective coating or layer 286 comprises or consists essentially of SiNₓC_{y}.

Optionally for any of the embodiments, the precursor comprises a monocyclic siloxane, a polycyclic siloxane, a polysilsesquioxane, a monocyclic silazane, a polycyclic silazane, a polysilsesquiazane, a silatrane, a silquasilatrane, a silproatrane, an azasilatrane, an azasilquasiatrane, an azasilproatrane, or a combination of any two or more of these precursors.

Optionally for any of the embodiments, the precursor comprises a monocyclic siloxane, a polycyclic siloxane, a polysilsesquioxane, a Silatrane, a Silquasilatrane, a Silproatrane, or a combination of any two or more of these precursors. Optionally for any of the embodiments, the precursor comprises octamethylcyclotetrasiloxane (OMCTS) or consists essentially of OMCTS. Other precursors described elsewhere in this specification or known in the art are also contemplated for use according to the disclosure.

Optionally for any of the embodiments, the pH protective coating or layer 286 as applied is between 10 and 1000 nm thick, optionally between 50 and 800 nm thick, optionally between 100 and 700 nm thick, optionally between 300 and 600 nm thick. The thickness does not need to be uniform throughout the vessel, and will typically vary from the preferred values in portions of a vessel.

Optionally for any of the embodiments, the pH protective coating or layer 286 contacting the fluid 218 is between 10 and 1000 nm thick, optionally between 50 and 500 nm thick, optionally between 100 and 400 nm thick, optionally between 150 and 300 nm thick two years after the pharmaceutical package 210 is assembled.

Optionally for any of the embodiments, the rate of erosion of the pH protective coating or layer 286, if directly contacted by a fluid 218 having a pH of 8, is less than 20%, optionally less than 15%, optionally less than 10%, optionally less than 7%, optionally from 5% to 20%, optionally 5% to 15%, optionally 5% to 10%, optionally 5% to 7%, of the rate of erosion of the barrier coating 288, if directly contacted by the same fluid 218 under the same conditions.

Optionally for any of the embodiments, the pH protective coating or layer 286 is at least coextensive with the barrier coating 288. The pH protective coating or layer 286 alternatively can be less extensive than the barrier coating, as when the fluid does not contact or seldom is in contact with certain parts of the barrier coating absent the pH protective coating or layer. The pH protective coating or layer 286 alternatively can be more extensive than the barrier coating, as it can cover areas that are not provided with a barrier coating.

Optionally for any of the embodiments, the pharmaceutical package 210 can have a shelf life, after the pharmaceutical package 210 is assembled, of at least one year, alternatively at least two years.

Optionally for any of the embodiments, the shelf life is measured at 3° C., alternatively at 4° C. or higher, alternatively at 20° C. or higher, alternatively at 23° C., alternatively at 40° C.

Optionally for any of the embodiments, the pH of the fluid 218 is between 5 and 6 and the thickness by TEM of the pH protective coating or layer 286 is at least 80 nm at the end of the shelf life. Alternatively, the pH of the fluid 218 is between 6 and 7 and the thickness by TEM of the pH protective coating or layer 286 is at least 80 nm at the end of the shelf life. Alternatively, the pH of the fluid 218 is between 7 and 8 and the thickness by TEM of the pH protective coating or layer 286 is at least 80 nm at the end of the shelf life. Alternatively, the pH of the fluid 218 is between 8 and 9 and the thickness by TEM of the pH protective coating or layer 286 is at least 80 nm at the end of the shelf life. Alternatively, the pH of the fluid 218 is between 5 and 6 and the thickness by TEM of the pH protective coating or layer 286 is at least 150 nm at the end of the shelf life. Alternatively, the pH of the fluid 218 is between 6 and 7 and the thickness by TEM of the pH protective coating or layer 286 is at least 150 nm at the end of the shelf life. Alternatively, the pH of the fluid 218 is between 7 and 8 and the thickness by TEM of the pH protective coating or layer 286 is at least 150 nm at the end of the shelf life. Alternatively, the pH of the fluid 218 is between 8 and 9 and the thickness by TEM of the pH protective coating or layer 286 is at least 150 nm at the end of the shelf life.

Optionally for any of the embodiments, the fluid 218 removes the pH protective coating or layer 286 at a rate of 1 nm or less of pH protective coating or layer thickness per 44 hours of contact with the fluid 218 (200 nm per year), alternatively 1 nm or less of pH protective coating or layer thickness per 88 hours of contact with the fluid 218 (100 nm per year), alternatively 1 nm or less of pH protective coating or layer thickness per 175 hours of contact with the fluid 218 (50 nm per year), alternatively 1 nm or less of pH protective coating or layer thickness per 250 hours of contact with the fluid 218 (35 nm per year), alternatively 1 nm or less of pH protective coating or layer thickness per 350 hours of contact with the fluid 218 (25 nm per year). The rate of removing the pH protective coating or layer can be determined by TEM from samples exposed to the fluid for known periods.

Optionally for any of the embodiments, the pH protective coating or layer 286 is effective to provide a lower frictional resistance than the uncoated article surface 254. Preferably the frictional resistance is reduced by at least 25%, more preferably by at least 45%, even more preferably by at least 60% in comparison to the uncoated article surface 254. For example, the pH protective coating or layer 286 preferably is effective to reduce the frictional resistance between a portion of the wall 214 contacted by the fluid 218 and a relatively sliding part 258 after the pharmaceutical package 210 is assembled. Preferably, the pH protective coating or layer 286 is effective to reduce the frictional resistance between the wall 214 and a relatively sliding part 258 at least two years after the pharmaceutical package 210 is assembled.

Optionally, in any embodiment the calculated shelf life of the blood sample collection tube (total Si/Si dissolution rate) is more than six months, or more than 1 year, or more than 18 months, or more than 2 years, or more than 2½ years, or more than 3 years, or more than 4 years, or more than 5 years, or more than 10 years, or more than 20 years. Optionally, in any embodiment the calculated shelf life of the blood sample collection tube (total Si/Si dissolution rate) is less than 60 years.

Any minimum time stated here can be combined with any maximum time stated here, as an alternative embodiment.

Optionally, in any embodiment the shelf life of at least six months, alternatively at least 12 months, alternatively at least 18 months, alternatively 24 months, measured at a temperature of 5 degrees Celsius.

Optionally, in any embodiment the water vapor transmission rate (WVTR) in units of mg of moisture per day (mg/day) less than 0.01 mg/day, alternatively less than 0.008 mg/day, alternatively less than 0.005 mg/day, or alternatively less than 0.003 mg/day, as measured with water vapor transmission rate (WVTR), using the formula:

$$WVTR = P \frac{( {p}_{2} - {p}_{1} )}{l} {e}^{- {E}_{A} / RT}$$

where P, is the permeability of water vapor, l, is the thickness, p₂, is the partial pressure of water vapor on one side of the film and p₁ is on the other side, E_{A} is the activation energy, R is the universal gas constant and Tis the temperature.

Optionally, in any embodiment the oxygen permeation rate of less than 0.00030/day, alternatively less than 0.00020/day, alternatively less than 0.00018/day, or alternatively less than 0.00018/day measured in a units of time⁻¹ of the article, as measured with oxygen transmission rate (OTR) constant (k_{OTR}), using the formula:

$${k}_{OTR} = \frac{RT}{V} {P}_{article}$$

where V is the BCT volume, P_{article}, is the oxygen permeation rate constant of the BCT (with units moles time⁻¹ pressure⁻¹), T is the absolute temperature, and R is the Universal gas constant.

Optionally, in any embodiment the tensile strength in a range of about 40 to about 80 MPa, alternatively a range of about 50 to about 70 MPa, alternatively a range of about 55 to about 65 MPa, alternatively a range of about 59 to about 63 MPa.

Optionally, in any embodiment the draw volume that retains at least 90% of an original draw volume for a shelf life of at least 24 months.

Optionally, in any embodiment the draw volume that retains at least 90% of an original draw volume for a shelf life of at least 24 months.

Optionally, in any embodiment the draw volume that retains at least 95% of an original draw volume for a shelf life of at least 24 months.

Even another embodiment is a medical or diagnostic kit including a vessel having a coating or layer as defined in any embodiment herein on a substrate as defined in any embodiment above. Optionally, the kit additionally includes a medicament or diagnostic agent which is contained in the vessel in contact with the coating or layer; and/or a hypodermic needle, double-ended needle, or other delivery conduit; and/or an instruction sheet.

The substrate can be a pharmaceutical package or another vessel, for protecting a compound or composition contained or received in the vessel with a coating or layer against mechanical and/or chemical effects of the surface of the uncoated substrate.

The substrate can be a pharmaceutical package or another vessel, for preventing or reducing precipitation and/or clotting of a compound or a component of the composition in contact with the inner or interior surface of the vessel. The compound or composition can be a biologically active compound or composition, for example, a medicament, for example, the compound or composition can comprise insulin, wherein insulin precipitation can be reduced or prevented. Alternatively, the compound or composition can be a biological fluid, for example, a bodily fluid, for example, blood or a blood fraction wherein blood clotting can be reduced or prevented.

A concern of converting from glass to plastic vessels centers around the potential for leachable materials from plastics. With plasma coating technology, the coatings or layers derived from non-metal gaseous precursors, for example, HMDSO or OMCTS or other organosilicon compounds, will itself contain no trace metals and function as a barrier to inorganic, metals and organic solutes, preventing leaching of these species from the coated substrate into vessel fluids. In addition to leaching control of plastic vessels, the same plasma coating or layer technology offers potential to provide a solute barrier to the plunger tip, typically made of elastomeric plastic compositions containing even higher levels of leachable organic oligomers and catalysts.

Moreover, a critical factor in the conversion from glass to plastic vessels will be the improvement of plastic oxygen and moisture barrier performance. The plasma coating technology is suitable to maintain the SiOₓ barrier coating or layer for protection against oxygen and moisture over an extended shelf life.

### Substrate

### Vessels Generally

A vessel with a coating or layer as described herein and/or prepared according to a method described herein can be used for reception and/or storage and/or delivery of a compound or composition. The compound or composition can be sensitive, for example, air-sensitive, oxygen-sensitive, sensitive to humidity and/or sensitive to mechanical influences. It can be a biologically active compound or composition, for example, a pharmaceutical preparation or medicament like insulin or a composition comprising insulin. In another aspect, it can be a biological fluid, optionally a bodily fluid, for example, blood or a blood fraction. In certain aspects of the present disclosure, the compound or composition can be a product to be administrated to a subject in need thereof, for example, a product to be injected, like blood (as in transfusion of blood from a donor to a recipient or reintroduction of blood from a patient back to the patient) or insulin.

A vessel as described herein and/or prepared according to a method described herein can further be used for protecting a compound or composition contained in its interior space against mechanical and/or chemical effects of the surface of the vessel material. For example, it can be used for preventing or reducing precipitation and/or clotting or platelet activation of the compound or a component of the composition, for example, insulin precipitation or blood clotting or platelet activation.

It can further be used for protecting a compound or composition contained in its interior against the environment outside of the vessel, for example by preventing or reducing the entry of one or more compounds from the environment surrounding the vessel into the interior space of the vessel. Such environmental compound can be a gas or liquid, for example an atmospheric gas or liquid containing oxygen, air, and/or water vapor.

A vessel with a trilayer coating, as described herein, can also be evacuated and stored in an evacuated state. For example, the trilayer coating or layer allows better maintenance of the vacuum in comparison to a corresponding vessel without a trilayer coating or layer. In one aspect of this embodiment, the vessel with a trilayer coating or layer is a blood collection tube. The tube can also contain an agent for preventing blood clotting or platelet activation, for example, EDTA or heparin.

### Basic Protocols for Forming and Coating Vessels

The vessels tested in the subsequent working examples were formed and coated according to the following exemplary protocols, except as otherwise indicated in individual examples. Particular parameter values given in the following basic protocols, for example, the electric power and gaseous reactant or process gas flow, are typical values. When parameter values were changed in comparison to these typical values, this will be indicated in the subsequent working examples. The same applies to the type and composition of the gaseous reactant or process gas.

In some instances, the reference characters and Figures mentioned in the following protocols and additional details can be found in U.S. Pat. No. 7,985,188.

Protocol for Coating Vessel Interior with SiOₓ

The apparatus and protocol generally, as found in U.S. Pat. No. 7,985,188, were used for coating vessel interiors with an SiOₓ barrier coating or layer, in some cases with minor variations. A similar apparatus and protocol were used for coating vessels with an SiOₓ barrier coating or layer, in some cases with minor variations.

Protocol for Coating Vessel Interior with OMCTS pH Protective Coating or Layer

Vessels already interior coated with a barrier coating or layer of SiOₓ, as previously identified, are further interior coated with a pH protective coating or layer as previously identified, generally following the protocols of U.S. Pat. No. 7,985,188 for applying the lubricity coating or layer, except with modified conditions in certain instances as noted in the working examples. The conditions given here are for a cyclic olefin copolymer (COC) vessel, and can be modified as appropriate for vessels made of other materials. The apparatus as generally shown in FIGS. 3 and 4 of U.S. Pat. No. 7,985,188 is used to hold a vessel with butt sealing at the base of the vessel. Additionally, a cap is provided that seals the end of the vessel (illustrated in present FIG. 3).

The vessel is carefully moved into the sealing position over the extended probe or counter electrode 108 of U.S. Pat. No. 7,985,188 and pushed against a plasma screen. The plasma screen is fit snugly around the probe or counter electrode 108 ensuring good electrical contact. The probe or counter electrode 108 is grounded to the casing of the RF matching network.

The gas delivery port 110 of U.S. Pat. No. 7,985,188 is connected to a manual ball valve or similar apparatus for venting, a thermocouple pressure gauge and a bypass valve connected to the vacuum pumping line. In addition, the gas system is connected to the gas delivery port 110 allowing the gaseous reactant or process gas, octamethylcyclotetrasiloxane (OMCTS) (or the specific gaseous reactant or process gas reported for a particular example) to be flowed through the gas delivery port 110 (under process pressures) into the interior of the vessel.

The gas system is comprised of a commercially available heated mass flow vaporization system that heats the OMCTS to about 100° C. The heated mass flow vaporization system is connected to liquid octamethylcyclotetrasiloxane (Alfa Aesar® Part Number A12540, 98%). The OMCTS flow rate is set to the specific organosilicon precursor flow reported for a particular example. To ensure no condensation of the vaporized OMCTS flow past this point, the gas stream is diverted to the pumping line when it is not flowing into the interior of the COC vessel for processing.

Once the vessel is installed, the vacuum pump valve is opened to the vessel holder 50 and the interior of the COC vessel of U.S. Pat. No. 7,985,188. A vacuum pump and blower comprise the vacuum pump system. The pumping system allows the interior of the COC vessel to be reduced to pressure(s) of less than 100 mTorr while the gaseous reactant or process gases is flowing at the indicated rates.

Once the base vacuum level is achieved, the vessel holder 50 assembly is moved into the electrode 160 assembly. The gas stream (OMCTS vapor) is flowed into the gas delivery port 110 (by adjusting the 3-way valve from the pumping line to the gas delivery port 110. The pressure inside the COC vessel is approximately 140 mTorr as measured by a capacitance manometer (MKS) installed on the pumping line near the valve that controls the vacuum. In addition to the COC vessel pressure, the pressure inside the gas delivery port 110 and gas system is also measured with the thermocouple vacuum gauge that is connected to the gas system. This pressure is typically less than 6 Torr.

Once the gas is flowing to the interior of the COC vessel, the RF power supply is turned on to its fixed power level. A 600 Watt RF power supply is used (at 13.56 MHz) at a fixed power level indicated in a specific example. The RF power supply is connected to an auto-match, which matches the complex impedance of the plasma (to be created in the vessel) to the output impedance of the RF power supply. The forward power is as stated and the reflected power is 0 Watts so that the stated power is delivered to the interior of the vessel. The RF power supply is controlled by a laboratory timer and the power on time set to 10 seconds (or a different time stated in a given example).

Upon initiation of the RF power, a uniform plasma is established inside the interior of the vessel. The plasma is maintained for the entire coating time, until the RF power is terminated by the timer. The plasma produces a pH protective coating or layer on the interior of the vessel.

After pH protective coating, the gas flow is diverted back to the vacuum line and the vacuum valve is closed. The vent valve is then opened, returning the interior of the COC vessel to atmospheric pressure (approximately 760 Torr). The treated vessel is then carefully removed from the vessel holder 50 assembly (after moving the vessel holder 50 assembly out of the electrode 160 assembly).

A similar protocol is used, except using apparatus generally like that of FIG. 1 of U.S. Pat. No. 7,985,188, for applying a pH protective coating or layer to vessels.

### Protocol for Total Silicon Measurement

This protocol is used to determine the total amount of silicon coatings present on the entire vessel wall. A supply of 0.1 N potassium hydroxide (KOH) aqueous solution is prepared, taking care to avoid contact between the solution or ingredients and glass. The water used is purified water, 18 MD quality. A Perkin Elmer Optima Model 7300DV ICP-OES instrument is used for the measurement except as otherwise indicated.

Each device (vessel, such as a vial, syringe, tube, or the like) to be tested and its closure (in the case of a vessel) or other closure are weighed empty to 0.001 g, then filled completely with the KOH solution (with no headspace), closed with the closure, and reweighed to 0.001 g. In a digestion step, each vessel is placed in an autoclave oven (liquid cycle) at 121° C. for 1 hour. The digestion step is carried out to quantitatively remove the silicon coatings from the vessel wall into the KOH solution. After this digestion step, the vessels are removed from the autoclave oven and allowed to cool to room temperature. The contents of the vessels are transferred into ICP tubes. The total Si concentration is run on each solution by ICP/OES following the operating procedure for the ICP/OES.

The total Si concentration is reported as parts per billion of Si in the KOH solution. This concentration represents the total amount of silicon coatings that were on the vessel wall before the digestion step was used to remove it.

The total Si concentration can also be determined for fewer than all the silicon layers on the vessel, as when an SiOₓ barrier layer is applied, an SiOₓC_{y} second layer (for example, a pH protective coating or layer) is then applied, and it is desired to know the total silicon concentration of just the SiOₓC_{y} layer. This determination is made by preparing two sets of vessels, one set to which only the SiOₓ layer is applied and the other set to which the same SiOₓ layer is applied, followed by the SiOₓC_{y} layer or other layers of interest. The total Si concentration for each set of vessels is determined in the same manner as described above. The difference between the two Si concentrations is the total Si concentration of the SiOₓC_{y} second layer.

### Protocol for Measuring Dissolved Silicon in a Vessel

In some of the working examples, the amount of silicon dissolved from the wall of the vessel by a test solution is determined, in parts per billion (ppb), for example to evaluate the dissolution rate of the test solution. This determination of dissolved silicon is made by storing the test solution in a vessel provided with an SiOₓ and/or SiOₓC_{y} coating or layer under test conditions, then removing a sample of the solution from the vessel and testing the Si concentration of the sample. The test is done in the same manner as the Protocol for Total Silicon Measurement, except that the digestion step of that protocol is replaced by storage of the test solution in the vessel as described in this protocol. The total Si concentration is reported as parts per billion of Si in the test solution

### Protocol for Determining Average Dissolution Rate

The average dissolution rates reported in the working examples are determined as follows. A series of test vessels having a known total silicon measurement are filled with the desired test solution analogous to the manner of filling the vessels with the KOH solution in the Protocol for Total Silicon Measurement. (The test solution can be a physiologically inactive test solution as employed in the present working examples or a physiologically active pharmaceutical preparation intended to be stored in the vessels to form a pharmaceutical package). The test solution is stored in respective vessels for several different amounts of time, then analyzed for the Si concentration in parts per billion in the test solution for each storage time. The respective storage times and Si concentrations are then plotted. The plots are studied to find a series of substantially linear points having the steepest slope.

The plot of dissolution amount (ppb Si) versus days decreases in slope with time, even though it does not appear that the Si layer has been fully digested by the test solution.

For the PC194 test data in Table 10 below, linear plots of dissolution versus time data are prepared by using a least squares linear regression program to find a linear plot corresponding to the first five data points of each of the experimental plots. The slope of each linear plot is then determined and reported as representing the average dissolution rate applicable to the test, measured in parts per billion of Si dissolved in the test solution per unit of time.

### Protocol for Determining Calculated Oxygen Transmission Rate

The oxygen transmission rate (OTR) reported in the working examples throughout this document are determined by measuring the oxygen partial pressure over time inside a stoppered blood sample collection tube with a Mocon (Brooklyn Park, MN) OpTech system. An oxygen sensor is affixed to the interior of a blood sample collection tube. The blood sample collection tube is sealed with epoxy and a glass slide under a reduced oxygen atmosphere (nominally 0.2 percent oxygen). Once the epoxy has set, the initial atmospheric composition within the blood sample collection tube is measured. The blood sample collection tubes are stored in a controlled temperature chamber at 25° C. As the oxygen content within the blood sample collection tube increases over time, the intensity of the light emitted by the sensor decreases in accordance with the Stern-Volmer relationship for fluorescence. The oxygen partial pressure, pₒ_{₂}, t, is measured once each day over the course of 7 days. Experimental plots of ln(pₒ_{_{2,outside}}−Pₒ_{₂},) vs t are linear with slope equal to the oxygen transmission rate constant of the article. The OTR constant, in units of day⁻¹, is a convenient metric that can be used to calculate the permeation rate, in units such as cubic centimeters of oxygen per day per package under specified conditions. The lower limit of detection (LOD) of the OTR test instrumentation is 0.00006 day⁻¹.

### Protocol for Determining Water Vapor Transmission Rate

The water vapor transmission rate (WVTR) reported in the working examples below are determined by measuring the water vapor transmission rate into blood sample collection tubes using a modified version of USP 671 test methodology. Each blood sample collection tube weight was measured on a microbalance with four significant digits. Four grams of 3A molecular sieve pellets (purchased from Delta Adsorbents) was weighed separately on a microbalance. Pellets were transferred into a blood sample collection tube, which was closed with a rubber stopper. The measured moisture weight loss of the blood sample collection tube was plotted over time. The slope of the linear regression was determined to be equal to the water vapor transmission rate (WVTR) in units of moisture per day (i.e. mg/day).

### Protocol for Determining Draw Volume

The draw volume of evacuated blood sample collection tube (BCTs) was conducted according to ISO 6710:1995 and ISO 6710:2017. The blood sample collection tube were stoppered under partial vacuum, stored at ambient temperature and tested at 1, 3, 6, 15 and 24 months intervals after the rubber stopper was inserted. A total of 16 blood sample collection tube (i.e. 4 BCTs from 4 different production lots) were measured at each time point.

### Protocol for Determining Drop Impact

The drop impact test for the blood sample collection tube (BCT) is determined as follows. A blood sample collection tube is filled with water to the desired fill volume (i.e. 9 ml or 4 ml) followed by rubber stopper insertion. A vacuum pump pulls a partial vacuum pressure of −26.8 in Hg for 9 ml BCTs and −26.0 in Hg for 4 ml BCTs. After 5 seconds of applied vacuum to the BCT, a rubber stopper is inserted. Each BCT was dropped in a vertical orientation inside a plexiglass tube to a height of 36 inches measured from the bottom of the tube to the surface of the floor. Each dropped BCT was visually inspected for cracks. If no cracks were observed, then the drop test was repeated with a new set of BCTs, but at the height of 48 inches. Visual inspection of cracks was repeated a second time.

### Protocol for Determining Calculated Shelf Life

The calculated shelf life values reported in the working examples below are determined by extrapolation of the total silicon measurements and average dissolution rates, respectively determined as described in the Protocol for Total Silicon Measurement and the Protocol for Determining Average Dissolution Rate. The assumption is made that under the indicated storage conditions, the SiOₓC_{y} pH protective coating or layer will be removed at the average dissolution rate until the coating is entirely removed. Thus, the total silicon measurement for the vessel, divided by the dissolution rate, gives the period of time required for the test solution to totally dissolve the SiOₓC_{y} coating. This period of time is reported as the calculated shelf life. Unlike commercial shelf life calculations, no safety factor is calculated. Instead, the calculated shelf life is the calculated time to failure.

It should be understood that because the plot of ppb Si versus hours decreases in slope with time, an extrapolation from relatively short measurement times to relatively long calculated shelf lives is believed to be a “worst case” test that tends to underestimate the calculated shelf life actually obtainable.

### TEST METHODS

### Barrier Improvement Factor

The barrier improvement factor (BIF) of a barrier coating or layer can be determined by providing two groups of identical containers, adding a barrier layer to one group of containers, testing a barrier property (such as the rate of outgassing in micrograms per minute or another suitable measure) on containers having a barrier, doing the same test on containers lacking a barrier, and taking a ratio of the properties of the materials with versus without a barrier. For example, if the rate of outgassing through the barrier is one-third the rate of outgassing without a barrier, the barrier has a BIF of 3.

### Measurement of Coating Thickness

The thickness of a PECVD coating or layer such as the pH protective coating or layer, the barrier coating or layer, the lubricity coating or layer, and/or a composite of any two or more of these layers can be measured, for example, by transmission electron microscopy (TEM). An exemplary TEM image for a pH protective coating or layer is shown in FIG. 18. An exemplary TEM image for an SiOₓ barrier coating or layer also is shown in FIG. 18.

The TEM can be carried out, for example, as follows. Samples can be prepared for Focused Ion Beam (FIB) cross-sectioning in two ways. Either the samples can be first coated with a thin layer of carbon (50-100 nm thick) and then coated with a sputtered coating or layer of platinum (50-100 nm thick) using a K575X Emitech primer coating or layer system, or the samples can be coated directly with the protective sputtered Pt layer. The coated samples can be placed in an FEI FIB200 FIB system. An additional coating or layer of platinum can be FIB-deposited by injection of an organometallic gas while rastering the 30 kV gallium ion beam over the area of interest. The area of interest for each sample can be chosen to be a location half way down the length of the vessel. Thin cross sections measuring approximately 15 μm (“micrometers”) long, 2 μm wide and 15 μm deep can be extracted from the die surface using an in-situ FIB lift-out technique. The cross sections can be attached to a 200 mesh copper TEM grid using FIB-deposited platinum. One or two windows in each section, measuring about 8 μm wide, can be thinned to electron transparency using the gallium ion beam of the FEI FIB.

Cross-sectional image analysis of the prepared samples can be performed utilizing either a Transmission Electron Microscope (TEM), or a Scanning Transmission Electron Microscope (STEM), or both. All imaging data can be recorded digitally. For STEM imaging, the grid with the thinned foils can be transferred to a Hitachi HD2300 dedicated STEM. Scanning transmitted electron images can be acquired at appropriate magnifications in atomic number contrast mode (ZC) and transmitted electron mode (TE). The following instrument settings can be used.

| | Scanning Transmission |
|---|---|
| Instrument | Electron Microscope |
| Manufacturer/Model | Hitachi HD2300 |
| Accelerating Voltage | 200 kV |
| Objective Aperture | 2 |
| Condenser Lens 1 Setting | 1.672 |
| Condenser Lens 2 Setting | 1.747 |
| Approximate Objective | 5.86 |
| Lens Setting | |
| ZC Mode Projector Lens | 1.149 |
| TE Mode Projector Lens | 0.7 |
| Image Acquisition | |
| Pixel Resolution | 1280 × 960 |
| Acquisition Time | 20 sec.(×4 |

For TEM analysis the sample grids can be transferred to a Hitachi HF2000 transmission electron microscope. Transmitted electron images can be acquired at appropriate magnifications. The relevant instrument settings used during image acquisition can be those given below.

| Instrument | Transmission Electron Microscope |
|---|---|
| Manufacturer/Model | Hitachi HF2000 |
| Accelerating Voltage | 200 kV |
| Condenser Lens 1 | 0.78 |
| Condenser Lens 2 | 0 |
| Objective Lens | 6.34 |
| Condenser Lens Aperture | 1 |
| Objective Lens Aperture | 3 |
| for imaging | |
| Selective Area Aperture | N/A |
| for SAD | |

### SEM Procedure

SEM Sample Preparation: Each vessel sample was cut in half along its length (to expose the inner or interior surface). The top of the vessel (Luer end) was cut off to make the sample smaller.

The sample was mounted onto the sample holder with conductive graphite adhesive, then put into a Denton Desk IV SEM Sample Preparation System, and a thin (approximately 50 Å) gold coating was sputtered onto the inner or interior surface of the vessel. The gold coating is used to eliminate charging of the surface during measurement.

The sample was removed from the sputter system and mounted onto the sample stage of a Jeol JSM 6390 SEM (Scanning Electron Microscope). The sample was pumped down to at least 1×10−6 Torr in the sample compartment. Once the sample reached the required vacuum level, the slit valve was opened and the sample was moved into the analysis station.

The sample was imaged at a coarse resolution first, then higher magnification images were accumulated. The SEM images can be, for example, 5 μm edge-to-edge (horizontal and vertical).

### XPS

The composition of the SiOₓ or other barrier coating or layer can be measured, for example, by X-ray photoelectron spectroscopy (XPS).

### Silicon Dissolution Rate

As shown in the working examples, the silicon dissolution rate is measured by determining the total silicon leached from the vessel into its contents, and does not distinguish between the silicon derived from the pH protective coating or layer 286, the barrier coating or layer 288, or other materials present.

### Additional Experimental Testing Protocols

### Gas Permeation, Draw Volume and Shelf-Life of any Embodiment

### Theory of Operation

The inventors offer the following theory of operation of the blood sample collection tube described here. The disclosure is not limited by this theory's accuracy or to the embodiments predictable by use of this theory.

Shelf-life prediction of a blood sample collection tube (BCT) starts with an understanding of the relationship between the inside pressure before filling and the volume of liquid drawn into the tube after filling. First, consider an empty, evacuated and stoppered blood sample collection tube with a total internal pressure at the storage time just prior to liquid draw, Pₜₒₜₐₗ and a volume, Vₜₒₜₐₗ. Pₜₒₜₐₗ at this point is less than one atmosphere of pressure or a partial vacuum pressure. After puncturing the stopper and connecting it to a source of liquid at atmospheric pressure, Pₐₜₘ, the liquid flows freely into the blood sample collection tube until the pressure reaches a final headspace pressure of Pₕ. After the blood sample collection tube is filled, the headspace pressure above the liquid, Pₕ, is equal to the atmospheric pressure or draw pressure, P_{d}. (i.e., 1 atm). The total blood sample collection tube volume, Vₜ, is the sum of the final headspace volume, Vₕ and the liquid volume or liquid draw volume, V_{d}. Applying Boyles Law, PₜₒₜₐₗVₜₒₜₐₗ=PₕVₕ, substituting Pₕ=P_{d} and solving for the draw volume is as follows:

$${V}_{D} = {V}_{total} ( 1 - \frac{{P}_{total}}{{P}_{d}} ) \qquad ( 1 )$$

Formula 1 indicates that the draw volume is directly related to the total internal pressure in the blood sample collection tube. During storage, air slowly enters both glass and plastic stoppered blood sample collection tubes, which increases the total pressure over time. As the inside pressure increases, the amount of blood drawn in to the blood sample collection tube decreases. The change of the total internal pressure over time may be related to the steady-state gas permeation in the blood sample collection tube.

The instantaneous oxygen transmission through a sealed blood collection tube (BCT) is calculated using Formula 2:

$$\frac{{dp}_{{O}_{2}}}{dt} = \frac{RT}{V} {P}_{article} ( {p}_{{O}_{2} , outside} - {p}_{{O}_{2} , t} ) \qquad ( 2 )$$

where V is the BCT volume, P_{article}, is the oxygen permeation rate constant of the BCT (with units moles time⁻¹ pressure⁻¹), Pₒ_{_{2,outside}} is the partial pressure of oxygen outside the BCT, pₒ_{_{2,t}} is the partial pressure of oxygen inside the BCT at a particular time t, T is the absolute temperature, and R is the Universal gas constant. The change rate of oxygen partial pressure is not a constant of the system; as oxygen permeates into the BCT, pₒ_{_{2,t}} increases and the rate decreases.

Performing integration of formula (2) results in formula 3:

$$\frac{{p}_{{O}_{2} , out} - {p}_{{O}_{2} , t}}{{p}_{{O}_{2} , out} - {p}_{{O}_{2} , o}} = {e}^{- \frac{RT}{V} {P}_{article} t} \qquad ( 3 )$$

where pₒ_{_{2,0}} is the oxygen partial pressure in the article at time zero, and k_{OTR} is the oxygen transmission rate (OTR) constant (with units of time⁻¹) of the article. Experimental plots of ln(pₒ_{_{2,outside}}−Pₒ_{_{2,t}}) vs t are linear with slope:

$$slope = - \frac{RT}{V} {P}_{article} = - {k}_{OTR} \qquad ( 4 )$$

The permeation rate constant (P_{article}) and oxygen transmission rate (OTR) constant (k_{OTR}) are related by the following relationship:

$${k}_{OTR} = \frac{RT}{V} {P}_{article} \qquad ( 5 )$$

The OTR constant, k_{OTR}, is the time constant for equilibration of the inside oxygen partial pressure with the outside oxygen pressure. For example, the oxygen pressure differential is 37% of the starting differential after 1/k_{OTR}, and the inside oxygen partial pressure has equilibrated with the outside after about 5/k_{OTR}. The OTR constant can be used to calculate the permeation rate constant and the instantaneous OTR at any particular oxygen partial pressure differential. A lower OTR constant implies a better barrier to oxygen permeation.

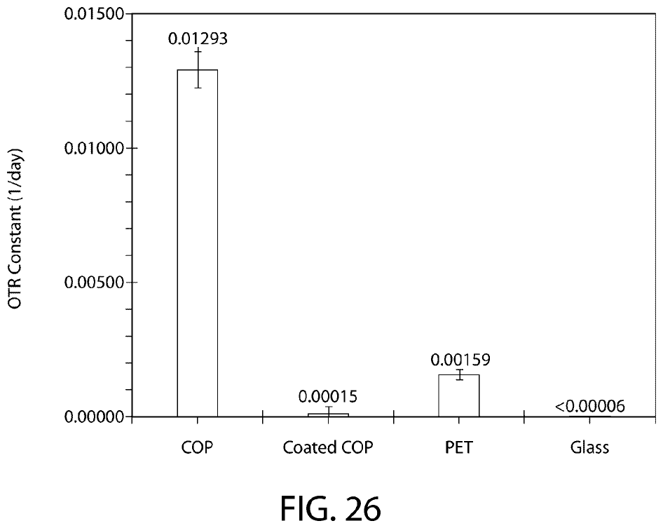

A comparison of the OTR constant for COP, barrier coated COP, PET and glass 9 ml BCTs are shown in FIG. 26. A plain COP BCT has the highest OTR constant of 0.0129 day⁻¹. A standard PET BCT has an OTR constant 8 times lower (i.e. 0.00159 day⁻¹) and a coated COP BCT has an OTR constant 86 times lower (i.e. 0.00015 day⁻¹) than an uncoated COP BCT. The application of the barrier coating to COP provides about 10 times lower OTR constant than the standard PET BCT. Since the relative shelf life of PET and coated COP BCTs is directly proportional to the ratio of their OTR constants, the COP BCT shelf-life is about 10 times longer than a PET BCT. A glass blood collection tube is below the lower detection limit of the test method or 0.00006 day⁻¹.

The permeation of air through the stopper and plastic BCT wall increases the internal pressure and ultimately decreases the draw volume over storage time. The relationship between draw volume and storage time can be derived by first realizing that the total internal pressure, Pₜₒₜₐₗ, in equation 1 is the sum of both nitrogen, p_{N}_{₂}, and oxygen, pₒ_{₂}, partial pressures inside the BCT. Solving for, Pₜₒₜₐₗ, in formula 6:

$${P}_{total} = {P}_{d} ( 1 - \frac{{V}_{D}}{{V}_{total}} ) = {p}_{{O}_{2}} + {p}_{{N}_{2}} \qquad ( 6 )$$

The permeation of nitrogen into the BCT follows a behavior analogous to formula 3, but with a nitrogen transmission rate constant, k_{N}_{₂}, that is approximately three to six times smaller (i.e. slower permeation rate) than oxygen for most polymer articles. Solving for both pₒ_{_{2,t}} and p_{N}_{_{2,t}} using formulas of the form of formula 3 and substituting into formula 6 results in the steady-state storage time dependence of the BCT internal pressure:

$${P}_{total} = {p}_{{N}_{2} , out} - ( {p}_{{N}_{2} , out} - {p}_{{N}_{2} , 0} ) {e}^{- {k}_{{N}_{2}} t} + ( {p}_{{O}_{2} , out} - {p}_{{O}_{2} , 0} ) {e}^{- {k}_{{O}_{2}} t} \qquad ( 7 )$$

This total internal pressure can then be used to estimate draw volume by substitution into formula 1. Although the change in internal pressure with relatively short storage times is approximately linear (using a Taylor series expansion approximation of formula 7), the internal pressure change is non-linear with longer storage times. Similarly, draw volume decay during storage is approximately linear at only short storage times.

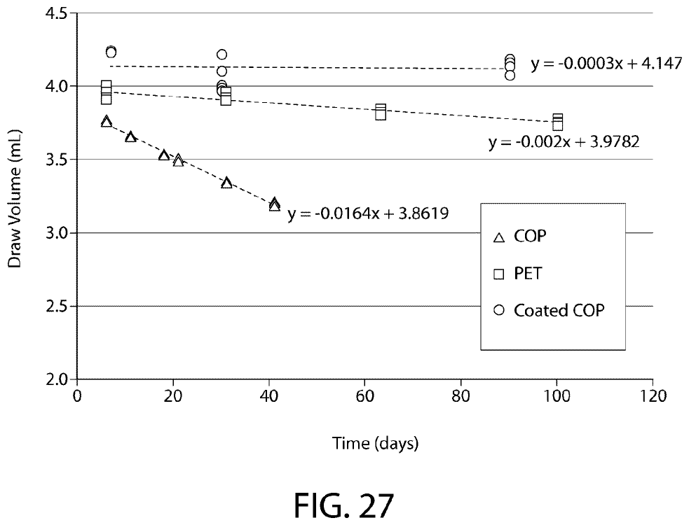

Draw volume was measured experimentally and plotted versus storage time for PET, COP and coated COP 4 ml BCTs. A linear regression analysis was applied to the data for each of the three BCT types as shown in FIG. 27. It should be noted that draw volume data for time zero was purposely omitted from the plot and the linear regression analysis. This is particularly important for the PET and COP BCTs because the measured draw volume at time zero (not shown) is much higher than an extrapolation of the linear regression analysis would predict. The reason for this is attributed to gas leaving the inside surface of the BCT and stopper while evacuated, also called degassing. The BCTs and stoppers are equilibrated with ambient air prior to evacuation, and as such, uncoated polymer, PET and stoppers are saturated with dissolved oxygen and nitrogen at the time of evacuation. After evacuation and stoppers inserted, the dissolved gas diffuses out into the evacuated interior of the BCT. Degassing can take several days to establish a steady-state gas permeation through the BCT wall and stopper. Coated COP BCTs are far less susceptible to degassing due to the dense barrier coating. Initial draw volumes were collected on day 7 in order to minimize the effects of degassing on the linear regression analysis.

For relatively short storage times shown in FIG. 27, the slope of the linear regression approximates the draw volume decay rate. The relative shelf life of two different BCT types can be approximated from the ratio of the draw volume decay rates. For example, the shelf life of a coated COP BCT is approximately 7 times longer than PET BCTs and 55 times longer than COP BCT.

The shelf life specifications for plastic blood collection tubes can vary depending on the manufacturer and materials of construction but are typically no more than 18 months. According to the draw volume decay in FIG. 27, 2-year shelf life is not achievable with a PET BCT due to its higher gas permeation. The specification for coated COP BCTs is a draw volume decay of no more than 10% over 2 years. In other words, if the BCT draw volume immediately after evacuation and stoppering is 4.0 ml, then after 2 years stored at ambient conditions, it cannot drop below 3.6 ml.

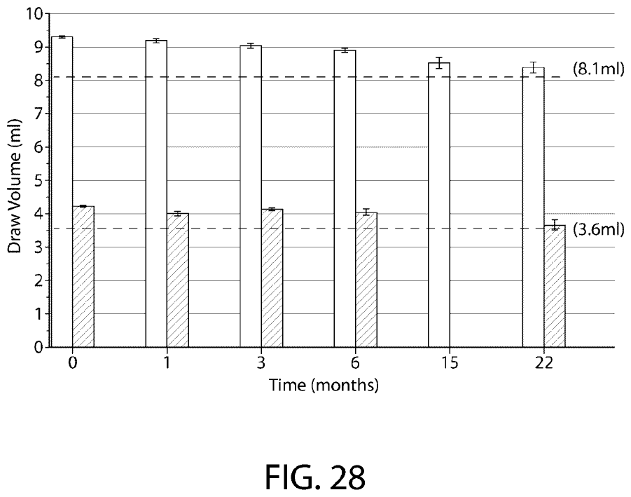

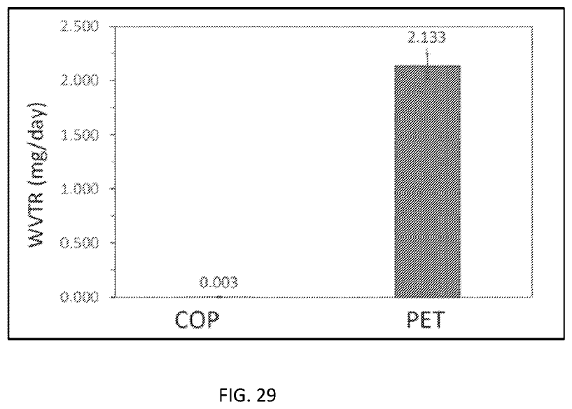

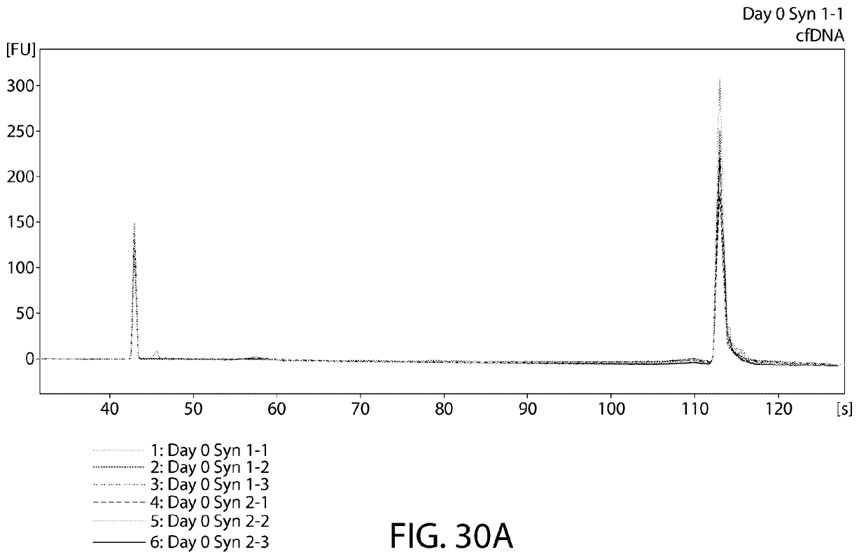

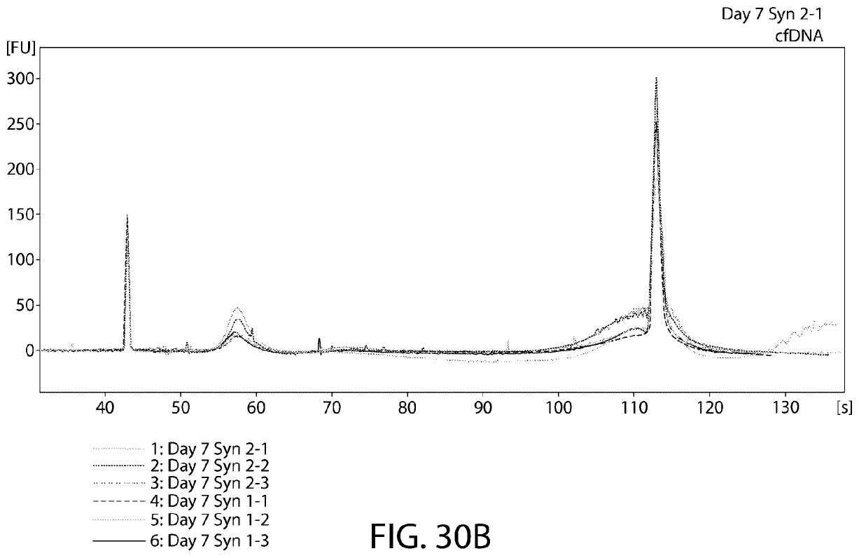

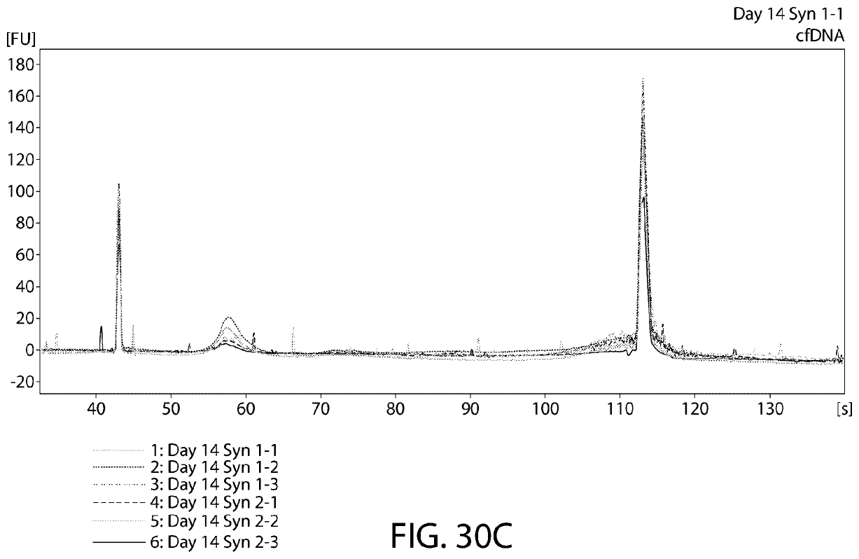

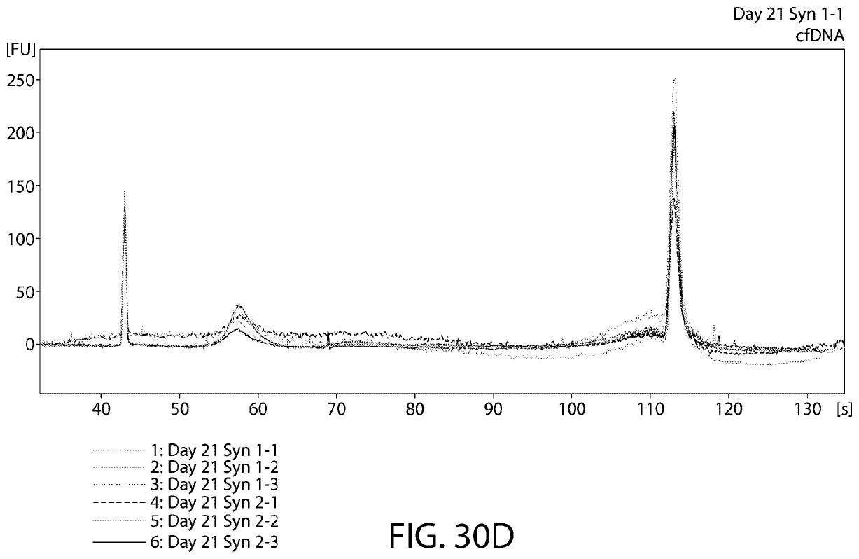

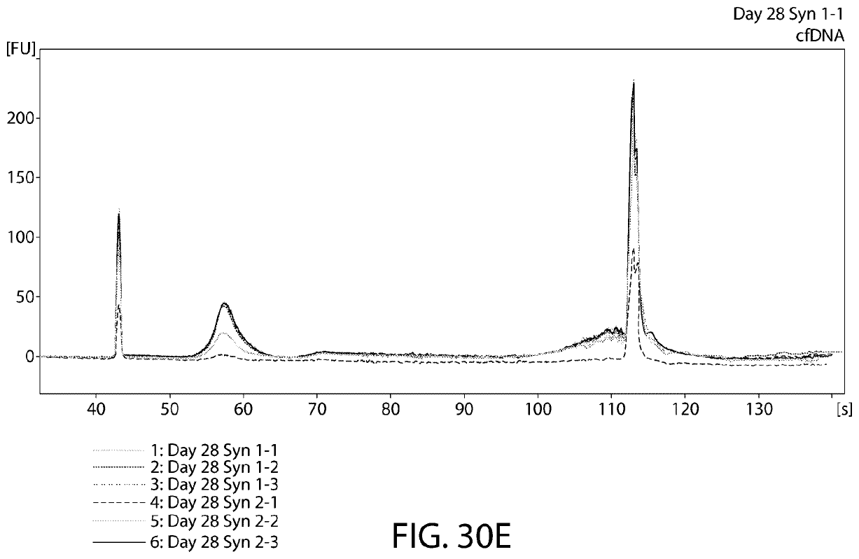

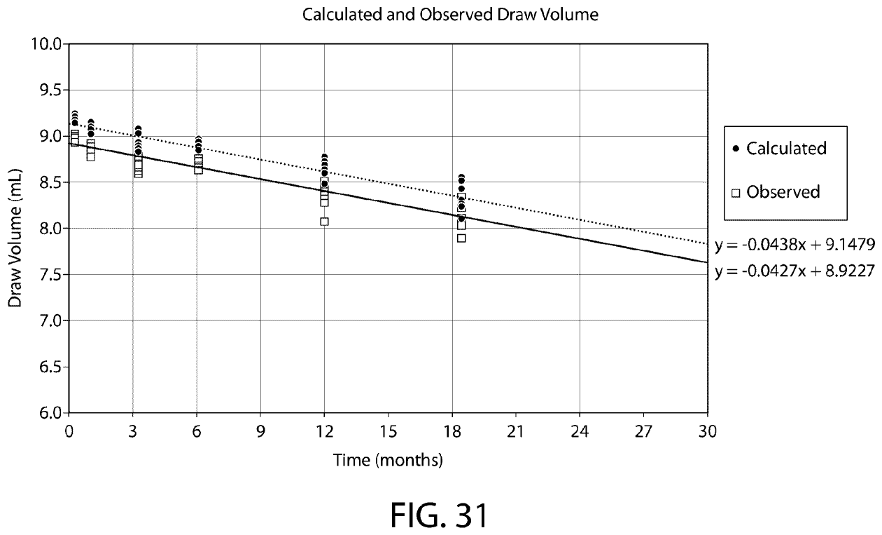

The draw volume decay of coated COP BCTs was measured over a period of 2 years. Two different BCT sizes with 9 ml and 4 ml nominal fill volumes were included in this testing. The targeted minimum draw volumes of 9 ml and 4 ml BCTs after 2 years was 8.1 ml and 3.6 ml, respectively. The bar graphs in FIG. 28 shows that the mean draw volumes are at or above the targeted draw volume minimums after 2 years of storage. Further, FIG. 31 shows the extrapolated draw volumes for the 9 ml blood collection tubes based on the observed draw volumes over a period of 30 months.

### Moisture Barrier and Absorption

The moisture barrier and absorption for the blood sample collection tube (BCT) is determined as an important metric. A moisture barrier and absorption for a blood sample collection tube is determined as follows to compare different materials. Low water vapor permeation is an important requirement of polymeric containers for preserving drug formulations stored in prefilled syringes, slowing evaporation from storage microplates and maintaining preservatives in blood collection tubes (BCTs). Moisture loss can result in gelation of the preservative and alter the appropriate ratio with blood serum, which affects the shelf life of the BCT. Water vapor permeability is a constant used to compare different materials without a geometry in mind.

Cyclic olefins exhibit superior water vapor permeability compared to other commodity polyolefins such as polypropylene (PP), and polyethylene terephthalate (PET). For example, the permeability of COP film is 0.25 g/m²/24 hr measured at 25° C. and 90% relative humidity (RH). PP and PET films of the same thickness have permeabilities of 0.5 and 1.9 g/m²/24 hr, respectively measured under the same temperature and RH conditions. The permeability, as with diffusivity, follows an Arrhenius temperature dependence whereby the permeability increases as the temperature increases. The extent of permeation and diffusion of water vapor at a higher temperature depends on the molecular structure and chemistry of the polymer. The water vapor permeability of COP film at 50° C. and 90% RH is 2 g/m²/24 hr. PP and PET films are 3 to 5 times more permeable measured at the same temperature and RH. COP, therefore, is a better material choice for safeguarding against water vapor permeability loss based on data from the literature.

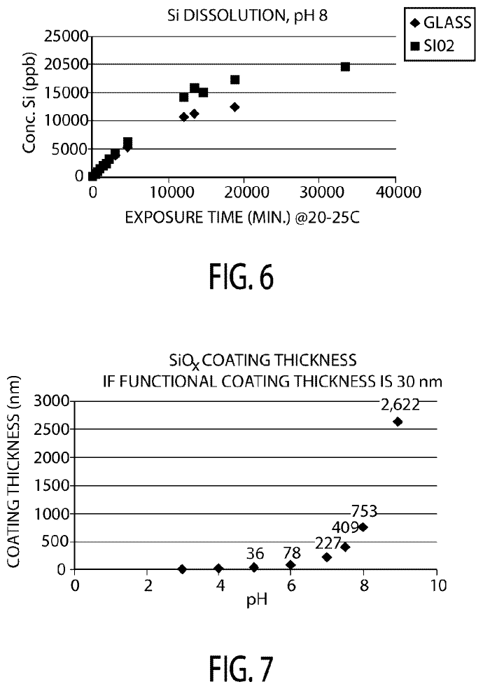

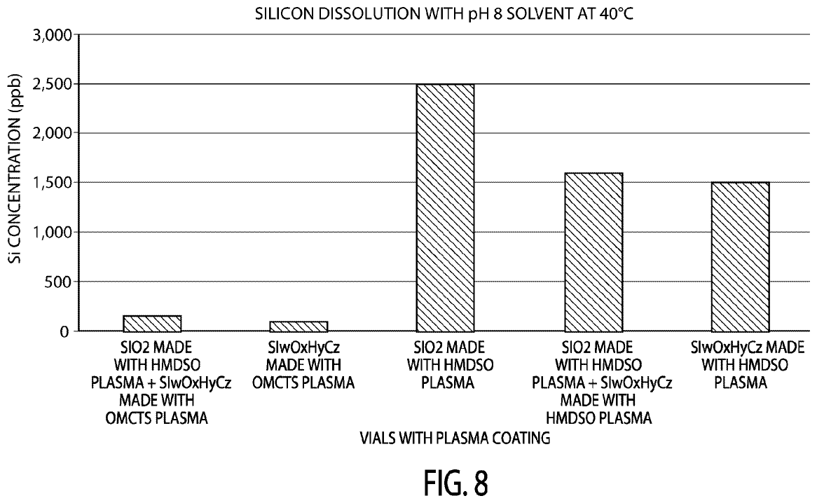

The water vapor transmission rate (WVTR) for the blood sample collection tube (BCT) is determined as an important metric. A water vapor transmission rate for a blood sample collection tube is determined as follows to compare different materials. The permeability constant, P, is a material-specific property independent of the geometry, which is related to WVTR by the following formula:

$$WVTR = P \frac{( {p}_{2} - {p}_{1} )}{l} {e}^{- {E}_{A} / RT} \qquad [ 8 ]$$

whereby, P, is the permeability of water vapor, l, is the thickness, p₂, is the partial pressure of water vapor on one side of the film and p₁ is on the other side, E_{A} is the activation energy, R is the universal gas constant and T is the temperature. The water vapor barrier of BCTs with different materials of construction was verified by measuring the water vapor transmission rate (WVTR). WVTR was measured according to USP 671 test method on plastic 4 mL blood collection tubes composed of COP and PET. Testing was conducted with a standard rubber stopper inserted into the BCT. COP BCTs exhibited over 700 times lower WVTR compared to PET as shown in FIG. 6. The WVTR of coated COP BCTs (data not shown) is essentially equivalent to COP BCTs. The barrier coating provides no additional water vapor barrier protection. Therefore the rate-limiting step of water vapor permeation is through the COP polymer. This is not the case for oxygen permeation, which is an ideal gas that follows Fick's Law of diffusion. Water vapor, by comparison, is a non-ideal gas that deviates from Fick's Law due to interactions with the oxide as it diffuses through the silicon oxide coating.

### Breakage, Mechanical Toughness and Heat Resistance

The breakage, mechanical toughness and heat resistance of the blood sample collection tubes was evaluated. The undisputed Achilles heel of glass has long been breakage and is unlikely to change despite attempts to improve it. While fused quartz has remarkably high thermal shock resistance, it's mechanical toughness, as measured by its compressive and tensile strength, is inferior to borosilicate glass as shown in Table 12. As part of the testing and comparison of the mechanical strength of blood sample collection tubes, the pure silica, borosilicate, COP and PER were compared.

Pure silica is impractical for manufacturing high-volume commodity containers due to its extremely high glass transition temperature (T_{g}). The addition of metal oxides to pure silica (e.g. borosilicate glass) is essential to lower its T_{g} for easier forming and shaping into complex shaped containers. This comes with improvements in mechanical toughness, particularly its ability to resist compressive and tensile stress compared to fused quartz. Nonetheless, borosilicate glass breakage and shattering from manufacturing and dropping is an omnipresent problem. This type of failure is exacerbated by defects and flaws in borosilicate glass, which cause it to fail well below its theoretical mechanical limits shown in Table 12. For example, even a simple scratch on the surface of a glass container can concentrate the stress from an impact causing it to fracture or shatter.

Polymers, in general, are inherently elastic materials that are less susceptible to impact-induced breakage compared to borosilicate glass. Cyclic olefin polymers (COP) are no exception with a high elongation (i.e., 20%/), low tensile modulus and high impact strength, as shown in Table 12. COP's impact strength is even maintained at temperatures down to −70° C., which is advantageous for cold storage.

Drop impact testing was conducted on 10 ml COP blood collection tubes (BCTs) filled with 9 ml of water, evacuated and stoppered. BCTs were dropped at the height of 36 inches onto a hard laboratory floor. None of the 24 tested COP BCTs showed any visual signs of breakage or cracking after impact. Glass BCTs filled with water, evacuated, stoppered and dropped from 36 inches had only 3 passing tubes while the rest failed due to breakage. All the glass BCTs failed when dropped from a height of 48 inches, as shown in Table 11 below.

The results of the drop test are listed below in Table 11. Specifically, the number of breaks and passes for a COP BTC and a glass tube are shown below.

TABLE 11
Summary Table of Drop Test of the COP
and glass blood sample collection tubes

| | | |
|---|---|---|
| Drop Height | COP | Glass |

| (inches) | # Break | # Pass | # Break | # Pass |
|---|---|---|---|---|
| 36 | 0 | 24 | 21 | 3 |
| 48 | 0 | 24 | 24 | |

Additional testing was conducted to ensure durability of the BCTs. For example, extreme centrifuge testing was conducted to ensure the durability of water-filled BCTs. The testing included residual centrifugation forces (RCF) for blood plasma, were the centrifugation was conducted for 500 and 2000×g. Three (water-filled, evacuated and stoppered BCTs were centrifuged for 20 minutes at RCF of 3000× g according to ISO 6710 guidelines. After visual inspection, none of the BCTs showed signs of cracks or leakage. Extreme centrifugation of water-filled hybrid BCTs did not impose breakage of any kind.

Polyethyleneterephthalate (PET) has a comparable thermal expansion, tensile strength and impact strength to COP at room temperature, although its glass transition temperature and heat deflection temperature is much lower, as shown in Table 12 below. COP's higher heat deflection temperature helps maintain mechanical strength and reduces the risk of part deformation at high temperatures. For example, COP maintains its dimensional integrity after 20 minutes of steam sterilization at 121° C., which is not possible for most polymers, including PET. Overall, COP can be utilized over a wider working temperature range compared to PET due to its excellent heat resistance and low-temperature toughness.

TABLE 12
Summary Table of Mechanical Toughness and Heat
Resistance of the blood sample collection tubes

| | | | Glass | Glass |
|---|---|---|---|---|
| Property | COP | PET | (silica) | (borosilicate) |
| Compressive | — | — | 1100 | 2000 |
| Strength (MPa) | | | | |
| Tensile Module (GPa) | 2.4 | 2.4 | 73 | 68 |
| Tensile Strength (MPa) | 61 | 56 | 50 | 280 |
| Thermal Expansion | 0.25 | 0.25 | 0.002 | 3.3-5.1 |
| (μm/m K) | | | | |
| Izod impact Strength | 32 | 66 | — | — |
| (J/m) @ 23° C. | | | | |
| Dupont Impact | 590 | — | — | 10 |
| Strength (J) | | | | |
| Glass Transition | 136 | 70 | 1900 | 560 |
| Temperature (° C.) | | | | |
| Deflection Temperature | 136 | 61 | | |
| (° C.) @ 1.8 MPa | | | | |

### REFERENCES

1) Bush V, Cohen R; The Evolution of Evacuated Blood Collection Tubes, Laboratory Medicine, 4(34), pgs 304-310, (2003).
2) Kleiner J J, Blood Collecting Apparatus, U.S. Pat. No. 2,460,641, (1945).
3) Bowen R A R, Ramaley A T. 2014. “Interferences from Blood Collection Tube Components on Clinical Chemistry Assays.” Biochemia Medica, 24 (1): 31-44.
4) Preissner C M, Reilly W M, Cyr R C, O'Kane D J, Singh R J, Grebe S K G, Plastic Versus Glass Tubes: Effects on Analytical Performance of Selected Serum and Plasma Hormone Assays, Clinical Chemistry, 50(7), 1245-1247 (2004).
5) Weikart C M, Pantano C G, Shallenberger J R, Performance Stability of Silicone Oxide Coated Plastic Parenteral Vials, PDA J Pharm Sci and Tech, 71 (4), 317-327 (2017)
6) Vieregg J R, Martin S J, Breeland A P, Weikart C M, Tirrell M V, Inhibiting Sterilization-induced Oxidation of Large Molecule Therapeutics Packaged in Plastic Parenteral Vials, PDA J Pharm Sci and Tech, 72 (1) 35-43 (2018)
7) Kinney S, Turney K, Sagona P, Part I of II: Improved Parenteral Containers Through Plasma Deposition of High Purity Glass, On Drug Delivery, Frederic Furness Publishing, (2014).
8) Kinney S, Weikart C, Sagona P, Part II of II: Improved Parenteral Containers through Plasma Deposition of High Purity Glass, On Drug Delivery, Frederic Furness Publishing, (2014).
9) Krenz H, Beyond Glass: Next Generation of Pharmaceutical Packaging, Pharmapack Conference, Paris, Feb. (2016)
10) Zeon Corporation, Zeonex Cyclic Olefin Polymer (COP), www.zeonex.com
11) Weikart C, Saaler-Reinhardt S, Glass Like Inner Barrier Coating Prevents Contamination of Drug Products with Potential Impurities from Primary Containers Composed of COP; A Case Study, Extractables & Leachables Europe, Nov. (2016)
12) Abdellatief A, Welt B A, Comparison of New Dynamic Accumulation Method for Measuring Oxygen Transmission Rate of Packaging against the Steady-State Method Described by ASTM D3985, Packag Tech Sci, John Wiley & Sons Ltd (2012)
13) ASTM D7709, Standard Test Methods for Measuring Water Vapor Transmission Rate (WVTR) of Pharmaceutical Bottles and Blisters; published by ASTM International
14) Graff G L, Burrows P E, Williford R E, Praino R F., Barrier Layer Technology for Flexible Displays, Flexible Flat Panel Displays, edited by Crawford G P, John Wiley & Sons, Ltd. 57-77 (2005).
15) Komori T, Kobayashi H, Uyama H, SiOₓ Barrier Films for Flexible Display, AIMCAL Fall Technical Conference (2005)
16) Castner D G, Ratner B D, Biomedical Surface Science: Foundations to Frontiers, Surface Science, 500, 28-60 (2000)
17) Weikart C M, Matsuzawa Y, Winterton L, Yasuda H K, Evaluation of Plasma Polymer-coated Contact Lenses by Electro-chemical Impedance Spectroscopy. J Biomed Mater Res, 54; 4, 597-607 (2001).
18) Boutroy N, Pernel Y, Rius J M, Auger F, Bardeleben H J von, Cantin J L, Abel F, Zeinert A, Casiraghi C, Ferrari A C, Robertson J, Hydrogenated Amorphous Carbon Film Coating of PET Bottles for Gas Diffusion Barriers, Diamond & Related Materials, 15, 921-927 (2006).
19) Grunwald H, Production Equipment of Plasma Enhanced CVD Permeation Barrier Coating of PET Beverage Bottles, SVC 47^{th} Annual Technical Conference Proceedings, Dallas, 470-476 (2004)
20) Weikart C, Fisk T, Larive M, Advances in PECVD Barrier Coating Development for ISBM PP Containers, SVC 51^{st} Annual Technical Conference Proceedings, Chicago, 569-573 (2008)
21) Iacocca R, Told N, Allegeier M, Bustard B, Dong Xia, Foubert M, Hofer J, Peoples S, Shelbourn T, Factors Affecting the Chemical Durability of Glass used in Pharmaceutical Industry, AAPS PharmaSciTech (2010)
22) Zeon Chemical L.P. Technical Update: Cyclic Olefin Polymer (COP), PharmEd' 7^{th} Annual Prefilled Syringe Forum, May (2014).
23) Tropsha Y G, Harvey N G, Activated Rate Theory of Oxygen and Water Transport through Silicon Oxide/Poly(ethylene terephthalate) Composite Barrier Structures, J Phys Chem, 101, 2259-2266 (1997).
24) Erlat A G, Spontak R J, Clarke R P, Robinson T C, Haaland P D, Tropsha Y, Harvey N G, Vogler E A, SiOₓ Gas Barrier Coatings on Polymer Substrates: Morphology and Gas Transport Considerations, J Phys Chem B, 103, 6047-6055 (1999).
25) Schaut R A, Peanasky J S, Demartino S E, Schiefelbein S L, A New Glass Option for Parenteral Packaging, PDA J Pharm Sci and Tech, 68, 527-534 (2014).
26) Allan D C, Ellison A J, Gomez S, Glass with Compressive Surface for Consumer Applications, U.S. Pat. No. 8,561,429 B2, October 22 (2013).

1. A blood sample collection tube characterized by a draw volume of about 7.5-9.5 ml comprising:
a lumen defined at least in part by a wall having an internal and external surface and a coating set on the internal surface, the coating set comprising a tie coating or layer, a barrier coating or layer, and a pH protective coating or layer;
the tie coating or layer comprising SiOₓC_{y} or SiNₓC_{y} wherein x is from about 0.5 to about 2.4 and y is from about 0.6 to about 3, the tie coating or layer having an outer surface facing the internal wall surface of the lumen and the tie coating or layer having an interior surface facing the lumen;
the barrier coating or layer comprising SiOₓ, wherein x is from 1.5 to 2.9, the barrier coating or layer being from 2 to 1000 nm thick, the barrier coating or layer having an outer surface facing the interior surface of the tie coating or layer and the barrier coating or layer having an interior surface facing the lumen; and
the pH protective coating or layer comprising SiOₓC_{y} or SiNₓC_{y} wherein x is from about 0.5 to about 2.4 and y is from about 0.6 to about 3, the pH protective coating or layer having an interior surface facing the lumen and an outer surface facing the interior surface of the barrier coating or layer; and
one or more nucleic acid preservatives.

2. The blood sample collection tube of claim 1, wherein the pH protective coating or layer is characterized in that the rate of erosion of the pH protective coating or layer, if directly contacted by a fluid composition having a pH between about 5 and about 9, is less than the rate of erosion of the barrier coating or layer, if directly contacted by the fluid composition.

3-7. (canceled)

8. The blood sample collection tube of claim 1, wherein the tube contains about 1.4 ml of the one or more nucleic acid preservatives and is evacuated to about 25-26 mm Hg.

9. The blood sample collection tube of claim 1, wherein the one or more nucleic acid preservatives is selected from one or more of the group consisting of: ethylenediaminetetraacetic acid (EDTA), K₃EDTA, aurintricarboxylic acid, diazolidinyl urea, dimethoylol-5,5-dimethylhydantoin, dimethylol urea, 2-bromo-2-nitropropane-1,3-diol, oxazolidines, sodium hydroxymethyl glycinate, 5-hydroxymethoxymethyl-1-1aza-3,7-dioxabicyclo[3.3.0]octane, 5-hydroxymethyl-1-1 aza-3,7dioxabicyclo[3.3.0]octane, 5-hydroxypoly[methyleneoxy]methyl-1-1aza-3,7dioxabicyclo[3.3.0]octane, quaternary adamantine, [ethylenebis(oxyethylenenitrilo)]tetraacetic acid (EGTA), 1,2-bis(2-aminophenoxy) ethane-N,N,N′,N′-tetraacetic acid (BAPTA), glycine, imidazolidinylurea, glutathione, lithium chloride, guanidine hydrochloride, urea, spermidine, biuret, and the compositions referred to in International Patent Application PCT/US2011/045405, U.S. Patent Application U.S. Pat. No. 9,012,135, and in U.S. Patent Published Application US 2020/0224189 A1.

10. The blood sample collection tube of claim 19, wherein the one or more nucleic acid preservatives is a composition comprising:
(a) a reducing agent;
(b) a first chaotropic substance;
(c) a second chaotropic substance;
(d) a third chaotropic substance;
(e) a first polyamine substance;
(f) a second polyamine substance; and
(g) a chelating agent.

11. The blood sample collection tube of claim 10, wherein the reducing agent is glutathione, the first chaotropic substance is LiCI, the second chaotropic substance is guanidine hydrochloride, the third chaotropic substance is urea, the first polyamine substance is spermidine; the second polyamine substance is biuret, and the chelating agent is EDTA.

12. The blood sample collection tube of claim 11, wherein the nucleic acid preservative is a composition comprising:
(a) glutathione in an amount from about 10 mM to about 200 mM;
(b) LiCI in an amount of from about 1 M to about 4 M;
(c) guanidine hydrochloride in an amount from about 0.1 M to about 0.9 M;
(d)urea in an amount from about 2 M to about 12 M;
(e) spermidine in an amount from about 10 μM to about 300 μM;
(f) biuret in an amount of from about 10 mM to about 100 mM; and
(g) EDTA in an amount of from about 1 mM to about 200 mM.

13. The blood sample collection tube of claim 1, wherein the one or more nucleic acid preservatives is a composition comprising:
(a) at least one volume excluding polymer;
(b) at least one osmotic agent;
(c) at least one enzyme inhibitor; and
(d) optionally, a metabolic inhibitor.

14-17. (canceled)

18. The blood sample collection tube of claim 13, wherein the nucleic acid preservative is a composition comprising:
(a) the at least one volume excluding polymer is polyethylene glycol (PEG);
(b) the at least one osmotic agent is NaCl,
(c) the at least one enzyme inhibitor is EDTA or citrate, and
(d) the metabolic inhibitor, if present, is sodium azide.

19. The blood sample collection tube of claim 1, wherein the pH protective coating or layer as applied has a thickness of between 100 and 700 nm.

20. The blood sample collection tube of claim 1, wherein the pH protective coating or layer has a thickness of between 50 and 500 nm two years after the tube is assembled.

21. The blood sample collection tube of claim 2, wherein the rate of erosion of the pH protective coating or layer, if directly contacted by a fluid composition having a pH of 8, is less than 20% of the rate of erosion of the barrier coating or layer, if directly contacted by the same fluid composition under the same conditions.

22. The blood sample collection tube of claim 2, wherein the rate of erosion of the pH protective coating or layer, if directly contacted by a fluid composition having a pH of 8, is from 5% to 20% of the rate of erosion of the barrier coating or layer, if directly contacted by the same fluid composition under the same conditions.

23. The blood sample collection tube of claim 1, wherein an FTIR absorbance spectrum of the pH protective coating or layer has a ratio greater than 0.75 between:
the maximum amplitude of the Si—O—Si symmetrical stretch peak between about 1000 and 1040 cm⁻¹, and
the maximum amplitude of the Si—O—Si asymmetric stretch peak between about 1060 and about 1100 cm⁻¹.

24. (canceled)

25. The blood sample collection tube of claim 1, wherein the silicon dissolution rate of the tube is less than 170 ppb/day as measured in a 50 mM potassium phosphate buffer diluted in water, adjusted to pH 8 with concentrated nitric acid, and containing 0.2 wt. % polysorbate-80 surfactant.

26-28. (canceled)

29. The blood sample collection tube of claim 1, wherein the pH protective coating or layer is characterized by an O-Parameter of less than 0.4, as measured with attenuated total reflection (ATR), using the formula:
$$O - Parameter = \frac{Intensity at 1253 {cm}^{- 1}}{Maximum intensity in the range 1000 to 1100 {cm}^{- 1}} .$$

30. (canceled)

31. The blood sample collection tube of claim 1, wherein the pH protective coating or layer is characterized by an N-Parameter of less than 0.7, as measured with attenuated total reflection (ATR) using the formula:
$$N - Parameter = \frac{Intensity at 840 {cm}^{- 1}}{Intensity at 799 {cm}^{- 1}} .$$

32. (canceled)

33. The blood sample collection tube of claim 1, wherein x is between 0.5 and 1.5 and y is between 0.9 and 2 for the pH protective coating or layer.

34-46. (canceled)

47. The blood sample collection tube of claim 1, characterized by a water vapor transmission rate (WVTR) in units of mg of moisture per day (mg/day) less than 0.01 mg/day, alternatively less than 0.008 mg/day, alternatively less than 0.005 mg/day, or alternatively less than 0.003 mg/day, as measured with the water vapor transmission rate (WVTR), using the formula:
$$WVTR = P \frac{( {p}_{2} - {p}_{1} )}{l} {e}^{- {E}_{A} / RT}$$
where P, is the permeability of water vapor, l, is the thickness, p₂, is the partial pressure of water vapor on one side of the film and p₁ is on the other side, E_{A} is the activation energy, R is the universal gas constant and Tis the temperature.

48. The blood sample collection tube of claim 1, characterized by an oxygen permeation rate of less than 0.00030/day, alternatively less than 0.00020/day, alternatively less than 0.00018/day, or alternatively less than 0.00018/day measured in a units of time−1 of the article, as measured with oxygen transmission rate (OTR) constant (k_{OTR}), using the formula:
$${k}_{OTR} = \frac{RT}{V} {P}_{article}$$
where V is the BCT volume, P_{article}, is the oxygen permeation rate constant of the BCT (with units moles time⁻¹ pressure⁻¹), T is the absolute temperature, and R is the Universal gas constant.

49-62. (canceled)
